(19) United States
(12) Patent Application Publication
(10) Pub. No.: US 2023/0159610 A1
(43) Pub. Date: May 25, 2023

(54) PROTEIN COMPRISING AT LEAST ONE REGULATORY T CELL ACTIVATING EPITOPE

(71) Applicant: Biotest AG, Dreieich (DE)

(72) Inventors: Steffen KISTNER, Frankfurt/Main (DE); Jens DAUFENBACH, Mainz (DE); Christopher UNGERER, Langen (DE); Peter HERBENER, Kirchhain (DE); Holger WALLMEIER, Sulzbach (DE)

(21) Appl. No.: 17/914,739

(22) PCT Filed: Mar. 26, 2021
(86) PCT No.: PCT/EP2021/057949

§ 371 Date: Sep. 26, 2022

(30) Foreign Application Priority Data
Mar. 27, 2020 (EP) 20166161.8

(51) Int. Cl. C07K 14/705; C07K 16/18

(52) U.S. Cl. CPC C07K 14/70503; C07K 16/18; C07K 2317/52; C07K 2319/30

(57) ABSTRACT

The present invention relates to the field of immunology, in particular, to the field of modulation of immune responses, in particular, suppression of immune responses and/or induction of tolerance. It provides a tregitope (regulatory T cell activating epitope) carrying polypeptide based on sequences derived from the Fc part of human IgG, wherein said TCP comprises at least one tregitope heterologous to human IgG that is located within at least one of three specific sequence frames. The invention provides such polypeptides for multiple purposes, e.g., in monomeric or dimeric form, wherein both are optionally be linked to an agent, e.g., to which an immune response is to be modulated or suppressed, or co-administered to such an agent, or for use as a stand-alone therapeutic. Nucleic acids encoding the TCP of the invention, pharmaceutic compositions and uses of said TCP are also provided.

The present invention relates to the field of immunology, in particular, to the field of modulation of immune responses, in particular, suppression of immune responses and/or induction of tolerance. It provides a tregitope (regulatory T cell activating epitope) carrying polypeptide based on sequences derived from the Fc part of human IgG, wherein said TCP comprises at least one tregitope heterologous to human IgG that is located within at least one of three specific sequence frames. The invention provides such polypeptides for multiple purposes, e.g., in monomeric or dimeric form, wherein both are optionally be linked to an agent, e.g., to which an immune response is to be modulated or suppressed, or co-administered to such an agent, or for use as a stand-alone therapeutic. Nucleic acids encoding the TCP of the invention, pharmaceutic compositions and uses of said TCP are also provided.

### BACKGROUND

Regulatory T cell activating epitopes (tregitopes) are peptides originally found in the constant region of human and primate type G immunoglobulins (IgGs) that are able to activate regulatory T cells (L. Cousens, et al., Hum. Immunol. 75, 1139-1146 (2014); Y. Su, R. Rossi, et al. J. Leukoc. Biol. 94, 377-383 (2013); L. Cousens, et al., J. Clin. Immunol. 33 (Suppl 1), S43-S49 (2013)). Tregitopes have been identified by computational epitope mapping of human Ig molecule looking for consensus regions that bind to multiple HLA class II molecules (R. Caspi, Blood 112:3003-3004 (2008)). The presentation of tregitopes is human leukocyte antigen (HLA)-restricted, wherein tregitopes are presented by multiple HLA. Tregitopes are described to selective engage and activate pre-existing natural regulatory T cells leading to suppression of inflammation (De Groot et al. Blood 112(8):3303-3311 (2008)).

Tregitopes are short (generally 15 to 20 amino acids) and linear peptide sequences that bind to HLA and activate regulatory T cells. Tregitope sequences are highly conserved in similar autologous proteins. Almost all identified tregitopes exhibit single 9-mer sequences, which can be predicted by an EpiMatrix epitope prediction algorithm (disclosed in WO 2008/094538A2) to bind to at least four different HLA DR alleles. Such identified tregitopes are likely to be broadly recognized in the human population. T cells responding to tregitopes exhibit a T regulatory phenotype (CD4⁺ CD25⁺ FoxP3⁺).

The immunosuppressive and immune modulatory effects of tregitopes have been recently reviewed by Maddur et al. Trend in Immunology 38(11): 789-792 (2017), and the activating effect of tregitopes on regulatory T cells has been described (L. Cousens, et al., Hum. Immunol. 75, 1139-1146 (2014); Su et al. J. Leukoc. Biol. 94, 377-383 (2013); L. Cousens, et al., J. Clin. Immunol. 33 (Suppl 1), S43-S49 (2013)). Recent publications indicate that tregitopes are suitable for the treatment of allergy (De Groot et al., Blood 112(8): 3303-3311 (2008)), inflammatory colitis (Van der Marel et al. World J Gastroenterol. 18(32): 4288-4299 (2012)), type 1 diabetes (Su et al. J. Leukoc. Biol. 94, 377-383 (2013), Cousens et al. Journal of Diabetes Research, Volume 2013, Article ID 621693 (2013)), multiple sclerosis (Elyaman et al., Neurology Research International, Volume 2011, Article ID:256460 (2011)), and induction of tolerance (Cousens, et al., Hum. Immunol. 75, 1139-1146 (2014)).

WO 2008/094538 A2 discloses several specific tregitopes and their application in the treatment of allergy, transplantation, autoimmunity, diabetes, Hepatitis B infection, Systemic Lupus Erythematosus, Graves' disease, and autoimmune Thyroiditis. Tregitopes may be used e.g. as a means of treatment for conditions with undesired immune response.

WO 2006/036834 A2 discloses a molecule with a human IgG Fc domain comprising a pharmacologically active peptide in a loop region.

Until now, it has been difficult to make use of the advantageous properties of tregitopes. It has been notoriously difficult to produce tregitopes or proteins containing them. Thus, there is a strong need to develop means and methods to easily manufacture tregitopes or proteins containing them.

Generally, tregitopes could be provided by peptide synthesis or recombinant production. However, chemical peptide synthesis is not satisfying in view of the amounts of the peptides needed. Furthermore, tregitopes taken alone are not well-suited for therapeutic administration, for example due to short half-life of the peptides in the circulation. To our knowledge, any attempts to express more than two tregitope incorporated in or fused to another protein have been without much success. Cousens et al. (Albumin Delivery of Tregitope Peptides for Tolerance Induction in Autoimmunity and Inflammatroy Disease, AAPS May 2014) tested fusion proteins of human serum albumin linked to different numbers of tregitopes, wherein proteins with two and four tregitopes were analysed in detail. The version with 4 terminally fused tregitops was described to result in significant breakdown products and difficulties in purification.

Furthermore, it is desirable to incorporate tregitopes into potentially immunogenic proteins or peptides, in order to convey target-specific immunologic tolerance, so that recombinant production would be desirable.

Still, recombinant production of tregitopes or proteins containing tregitopes is difficult. There have been attempts to produce tregitopes by bacterial expression systems (E. coli), but to our knowledge, all such these attempts failed, possibly due to high hydrophobicity of the peptides. Expression of tregitopes in eukaryotic systems also encountered similar problems. Approaches have been made to fuse tregitopes with albumin and other proteins in order to obtain improved expression. Again, to our knowledge these approaches did not lead to satisfying results.

Although it is possible to produce tregitopes by chemical synthesis (such as FMOC), it remains difficult to produce large quantities of tregitopes at reasonable costs or to routinely express tregitopes in fusion proteins by recombinant methods.

Thus, there is a strong need in the art for an approach which enables effective production and especially an efficient expression system for tregitopes, e.g., linking more than one tregitope to a target protein in order to convey target-specific immunologic tolerization. There is also a strong need to enable administration of tregitopes to a subject in order to take advantage of their therapeutic potential.

### THE PRESENT INVENTION

These problems are solved by the present invention as disclosed herein, e.g., by the subject-matter of the claims.

Tregitope Carrying Polypeptides (TCPs)

In a first embodiment, the invention provides a tregitope carrying polypeptide (TCP) comprising an amino acid sequence having at least 85% sequence identity with amino acids 135 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. SEQ ID NO: 1 represents the constant region of the heavy chain sequence of a human IgG1 (details described further below). Accordingly, amino acids 135 to 330 of SEQ ID NO: 1 comprises parts of the CH2 and CH3 domain of human IgG, and in particular comprises the disulfide bridge at C144. The TCP of the invention thus typically comprises sequences derived from the Fc-part of human IgG.

In a another embodiment, the invention provides a tregitope carrying polypeptide (TCP) comprising an amino acid sequence having at least 85% sequence identity with amino acids 114 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. Optionally, said TCP further comprises an amino acid sequence having at least 85% sequence identity with amino acids 135 to 330 of SEQ ID NO: 1.

In a another embodiment, the invention provides a tregitope carrying polypeptide (TCP) comprising an amino acid sequence having at least 85% sequence identity with amino acids 104 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. Optionally, said TCP further comprises an amino acid sequence having at least 85% sequence identity with amino acids 135 to 330 of SEQ ID NO: 1. Optionally, said TCP further comprises an amino acid sequence having at least 85% sequence identity with amino acids 114 to 330 of SEQ ID NO: 1.

In the course of the present invention a novel tregitope carrying polypeptide (abbreviated “TCP”) was developed, allowing expression and administration of tregitopes (equally termed “regulatory T cell activating epitopes”) in a particularly efficient and flexible manner for different purposes and applications.

The invention is based on the unexpected finding that tregitopes can be surprisingly well expressed if they are incorporated into the chain(s) of an immunoglobulin Fc-part (such as disclosed in SEQ ID NO: 1). Moreover, the use of a Fc-part chain as a backbone or carrier molecule for the tregitopes allows integration and successful expression of more than one tregitope, thereby providing a very efficient expression and/or delivery tool.

Furthermore, the inventors have identified particularly advantageous frames within said Fc-part chain which allow for particularly efficient expression of tregitopes. These frames provide a modular design allowing multiple variants of tregitopes and combinations thereof to be incorporated, thus providing enormous flexibility to design a product of choice.

The tregitope carrying polypeptide of the invention is also useful in a pharmaceutical context, particularly for treating immunological disorders. For example, the tregitope carrying polypeptide can be administered as a stand-alone therapeutic, e.g. to treat excessive immune reaction. The fact that tregitopes are integrated into an Fc-part does not only allow easier manufacture compared to isolated tregitopes, but it may also serve to improve the plasma half-life of the product compared to administration of single tregitopes. Thus, the tregitope carrying polypeptide is very useful as a therapeutic or prophylactic agent.

The tregitope carrying polypeptide also allows tregitopes to be easily incorporated into and/or attached to other proteins, e.g. in form of fusion proteins. Thus, the invention provides a flexible platform to attach tregitopes to a protein of choice, reducing the need for experimentation where tregitopes can be integrated. The present approach also provides a new tool for administering tregitopes combined with or linked to certain agents, such as proteins or peptides, to which immunological tolerance is to be conveyed. This may be particularly useful in view of autoimmunity, allergy, other diseases, and in the context of the prevention or reduction of undesired immune responses against therapeutics. Furthermore, e.g., by means of antigen binding regions linked to the tregitope carrying polypeptide, said protein may be targeted to specific tissues or cells.

The invention allows expression of tregitopes in a carrier suitable for many applications. However, the inventive approach may also be used to effectively produce isolated tregitopes, wherein the tregitopes are expressed within the TCP. After expression of the TCP, the tregitopes may be excised from the TCP (or a protein comprising the TCP), and further purified. This allows for efficient manufacture and use of isolated tregitopes.

Without intending to be bound by any theory, it is possible that the inventive approach, namely the use of a Fc-part chain of an immunoglobulin as a carrier sequence, counteracts the tendency of the tregitopes to stick together, thus enabling efficient expression. The results of the present approach are especially unexpected and advantageous, because, as discussed above, to our knowledge, previous attempts to fuse multiple tregitopes to proteins were not very successful.

Thus, the use of an Fc-part chain as a backbone for integrating tregitopes allows for efficient cloning and expression, especially including secretion, of tregitopes in biological, especially in eukaryotic, expression systems.

The use of an immunoglobulin Fc-part chain as a carrier molecule for tregitopes allows the efficient cloning and expression of tregitopes, especially of two or more tregitopes, or advantageously, also of three or more tregitopes, which may be different or identical tregitopes, within one polypeptide. The resulting TCP of the invention are stable and easy to purify.

As shown in the examples of the present disclosure, the TCP according to the present invention showed good results with respect to immune modulatory activity. This was shown by the immune suppressive capacity of the TCP on proliferation and activation of effector CD4+ T cells across a wide range of donors representative of the nine major HLA-DRB1 supertypes.

Many further useful embodiments, advantages, and applications will become apparent from the description of the invention.

The term “sequence identity” as used throughout this specification is known by a skilled person. Generally, an amino acid sequence has “at least x % identity” with another amino acid sequence, when the sequence identity between those two aligned sequences is at least x % over the full length of said other amino acid sequence. Such global alignments can be performed using for example publicly available computer homology programs such as the “EMBOSS” Needle program provided at the EMBL homepage at http://www.ebi.ac.uk/Tools/psa/emboss_needle/, using the following settings provided: MATRIX: BLOSUM 62; GAP OPEN 20; GAP EXTEND 0.5; OUTPUT FORMAT: pair; END GAP PENALTY: false; END GAP OPEN: 10; ENDGAP EXTEND: 0.5. Further methods of calculating sequence identity or sequence similarity/sequence homology percentages of sets of amino acid sequences are known in the art.

Insofar as specific regions, the frames defined herein are not taken into account for determining sequence identity, this means that, before the comparison for determining sequence identity is carried out, the respective subsequences of the frames are deleted both in the sequence with which the comparison is to be done and in the sequence to be compared. Here, in case of doubt, first an alignment over the full length sequences is carried out, and then the sequences corresponding to the frames in the comparative sequence are deleted. Additionally, and for the sake of clarity, any N-terminal and C-terminal subsequences outside of the core sequence relevant for the sequence identity as defined elsewhere in this document, are also not taken into account for determining the sequence identity. Thus, the said first alignment may also be used to identify and further eliminate such N-terminal and C-terminal subsequences not taken into account for calculating the sequence identity.

The present invention further provides a TCP comprising an amino acid sequence having at least 90%, at least 95%, at least 99% or 100% sequence identity with amino acids 135 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity.

The TCP of the present invention may also comprise an amino acid sequence having at least 85% sequence identity with amino acids 114 to 330 of SEQ ID NO: 1 (the sequence comprising the complete CH2 and CH3 domain of human IgG), wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. In this embodiment, the amino acid sequence identity for the specified region may also be at least 90%, at least 95%, at least 99% or 100%.

The TCP of the present invention may also comprise an amino acid sequence having at least 85% sequence identity with amino acids 104 to 330 of SEQ ID NO: 1 (the sequence comprising the CH2 and CH3 domain and a part of the hinge region), wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. In this embodiment, the amino acid sequence identity for the specified region may also be at least 90%, at least 95%, at least 99% or 100%.

In a further embodiment, the TCP of the present invention may also comprise an amino acid sequence having at least 85% sequence identity with amino acids 1 to 330 of SEQ ID NO: 1 (the amino acid sequence of the constant regions of human IgG), wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. In this embodiment, the amino acid sequence identity to the specified region may also be at least 90%, at least 95%, at least 99% or 100%.

In another embodiment, the present invention provides a TCP comprising a contiguous sequence of at least 190 amino acids having at least 50%, preferably, at least 60% sequence or, more preferably, at least 65% identity to amino acids No. 135-330 of SEQ ID NO: 1, wherein said TCP comprises at least two regulatory T cell activating epitopes which are heterologous to said Fc-part chain, wherein said protein optionally does not comprise the VH domain and/or the CH1 domain of an antibody. Preferably, at least one, optionally, at least two of the tregitopes of said TCP is/are located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1.

In said embodiment, the sequences of the frames are taken into account for determination of sequence identity, which leads to the lower sequence identity compared to, e.g., the TCP defined above.

The present invention also provides a TCP comprising an immunoglobulin, e.g., IgG Fc-part chain modified by insertion of at least one, preferably two, three, or four heterologous tregitopes, wherein said TCP does not comprise the VH domain and/or the CH1 domain of an antibody.

It is clear for the skilled person that for all TCPs mentioned above, the preferred features disclosed herein apply analogously, including but not limited to possible or preferred Tregitopes, Fc-part chains, sequence frames, and the rules for integration and location. Analogously, multimers and fusion proteins comprising the TCP can be designed and manufactured.

SEQ ID NO: 1 corresponds to UNIPROT sequence P01857. It represents the constant region of a human IgG heavy chain and has the following characteristics (Giuntini et al., 2016. Clin Vaccine Immunol 23:698-706):
1) Hinge region: Positions 103-113
2) CH2 domain: Positions 114-223
3) CH3 domain: Positions 224-330
4) Cysteine residues for intermolecular Fc-part dimerization: Positions 109 and 112
5) Cysteine residues intramolecular disulfide bridge (CH2 domain): Pos. 144 and 204
6) Cysteine residues intramolecular disulfide bridge (CH3 domain): Pos. 250 and 308
7) Potential glycosylation site (Asn 297 according to Kabat numbering of antibodies): Position 180.

As mentioned, the invention is based on the novel concept of introducing tregitopes into a chain of an immunoglobulin Fc-part or a fragment thereof. The terms “immunoglobulin Fc-part chain” and “chain of an immunoglobulin Fc-part” or simply “Fc-part chain” as used throughout the present specification are understood by the person skilled in the art (see e.g. Schroeder, H. W., & Cavacini, L. (2010) Structure and function of Immunoglobulins, J Allergy Clin Immunol vol. 125(2), S41-S52). The term “Fc-part” is known to the skilled person. An Fc-part chain according to the present invention means one chain of the Fc-fragment dimer of an immunoglobulin, or a fragment thereof. For example, such fragment can be obtained as an immunoglobulin G (IgG), preferably a human IgG by digestion with papain. The corresponding amino acid and nucleic acid sequences are known. An example for an Fc-part chain is the human IgG1 Fc-part chain disclosed as part of SEQ ID NO: 1. The Fc-part chain can be a full-length Fc-part chain or it can be shorter. Preferably, the polypeptide used for the TCP should correspond at least to the CH2 and CH3 domain of an IgG, such as a human IgG, possibly including the hinge region of the Fc-part chain, or parts of the hinge region.

If the sequence identity required is met, the immunoglobulin sequences in the TCP of the invention may also be derived from a murine IgG. Preferably, they are derived from human IgG.

The immunoglobulin Fc-part chain according to amino acids 135 to 330 of SEQ ID NO: represents most of the CH2 and the CH3 domain of the human immunoglobulin G (IgG) excluding the hinge region of said immunoglobulin.

The terms “CH2 domain”, “CH3 domain” and “hinge region” are known to the skilled person (see e.g. Schroeder, H. W., & Cavacini, L. (2010) Structure and function of Immunoglobulins, J Allergy Clin Immunol vol. 125(2), S41-S52). As mentioned above, the respective domains can be found in SEQ ID NO: 1 as follows:

(a) Hinge region: Positions 103-113

(b) CH2 domain: Positions 114-223

(c) CH3 domain: Positions 224-330

Each CH region forms a rather conserved loop-like domain via intramolecular disulfide bonds. The CH2 domain of IgG plays an important role in mediating effector functions and preserving antibody stability. In an antibody, it is involved in weak interactions with another CH2 domain through sugar moieties. The N-linked glycosylation at Asn297 is conserved in mammalian IgGs as well as in homologous regions of other antibody isotypes.

While, in an antibody or Fc-part chain dimer, the CH2 domains interact with each other via the sugar moieties, the CH3 domains directly interact with each other, and thus also play an important role for dimerization. These constant regions are also important for the effector functions of an antibody, in particular, for binding to the Fc receptors.

The Fc-part chain used for generating the TCP is derived from SEQ ID NO: 1. More specifically, it is derived at least from amino acids 135 to 330 of SEQ ID NO: 1. If dimerisation is desired, the Fc-part chain may also include a hinge region such as specified by amino acids 103 to 113 of SEQ ID NO: 1 (core hinge region, cf. Giuntini et al., 2016), or a part thereof that allows for dimerization, e.g., the TCP may be derived from amino acids No. 104 to 330 of SEQ ID NO: 1. It may also be derived from amino acids 40 to 330 of SEQ ID NO: 1 or 1-330 of SEQ ID NO: 1. “Derived” means that one or more modifications may be performed on the sequence.

One modification is the insertion or integration of at least one heterologous tregitope within the sequence.

If the tregitope carrying polypeptide retains the ability of an immunoglobulin Fc-part to bind to FcRn (neonatal Fc receptor), this may advantageously result in improvement of the half-life and stability of the TCP. Optionally, the glycosylation site is maintained for FcRn interactions. Optionally, the TCP of the present invention also binds to Fc-gammaRI, Fc-gammaRII and/or Fc-gammaRIII. In this case, a TCP derived from IgG should maintain the glycosylation site, as described above. Binding to Fc-gamma-Receptors may increase uptake by professional antigen-presenting cells, which may be advantageous in the context of the invention.

It is possible to introduce further mutations into the tregitope carrying polypeptide in order to alter or improve specific desired properties of the protein. Depending on the specific purpose of the TCP, in order to prevent or promote distinct effector functions mediated through the respective receptors, it may be e.g. preferred to inhibit the binding of the protein to neonatal Fc receptor or to Fc-gammaRI, Fc-gammaRII or Fc-gammaRII by introducing mutations to the relevant amino acids of the protein.

As the TCP typically is a soluble protein, it may advantageously be secreted by the cells expressing it. To this end, the TCP may comprise a signal sequence. The term “signal sequence” is generally known to the skilled person. More specifically, the term relates to a peptide linked, typically at the N-terminus, to the TCP, which promotes the intracellular transport and/or the secretion of the TCP. The signal sequence may be cleaved off during transport and secretion of the protein, or it may be removed, e.g., by separate enzymatic treatment. Examples for signal sequences include SEQ ID NO: 22.

Optionally, the TCP comprises a purification tag. The term “purification tag” is also understood by the skilled person. More specifically, the term relates to a peptide fused, typically at the N-terminus or C-terminus, to the TCP, facilitating purification of the synthesized TCP. Typical examples are a His-Tag, a FLAG-Tag, or Myc-Tag.

Moreover, TCP may also comprise post-translational modifications such as glycosylations, phosphorylations or PEGylations. Preferably, the TCP maintains the glycosylation site at Asn 297 according to Kabat numbering of antibodies), corresponding to position 180 in SEQ ID NO: 1.

One advantage of the TCP based on an immunoglobulin Fc-part is the long plasma half-life conveyed by the structure derived from an immunoglobulin Fc-part chain. However, the TCP may also comprise a half-life extending moiety, e.g. albumin, an albumin binding domain, or a Polyethylene Glycol (PEG) moiety.

Tregitopes

The term “tregitope” (or “regulatory T cell activating epitope”) as used throughout this invention is known to the person skilled in the art. Tregitopes are small linear peptides with a length of generally about 10 to 25 amino acids, e.g., about 15 to 20 amino acids, which are able to activate regulatory T cells. They have originally been identified in the constant region of human and primate IgG immunoglobulins (L. Cousens, et al., Hum. Immunol. 75, 1139-1146 (2014); Y. Su, R. Rossi, et al. J. Leukoc. Biol. 94, 377-383 (2013); L. Cousens, et al., J. Clin. Immunol. 33 (Suppl 1), S43-S49 (2013)). These short linear peptides are capable of activating regulatory T cells, in particular by binding to the MHC II pocket of the HLA complex on antigen-ic) presenting cells e.g. dendritic cells. Receptor-based interactions including the HLA-tregitope complex between antigen-presenting cells and regulatory T cells result in the activation of the latter cell type. Examples for T cell activating epitopes according to the present invention are given throughout this disclosure. Generally, sequences representing tregitopes are highly conserved in similar autologous proteins. Almost all identified tregitopes exhibit single 9-mer core sequences, which can be predicted by an EpiMatrix epitope prediction algorithm to bind to at least four different HLA DR alleles. Such identified tregitopes are likely to be broadly recognized in the human population. This selection is based on EpiMatrix score across HLA supertypes, validation of predicted hits in HLA binding assays, validation and supporting evidence, in vitro assays and in vivo models and context considerations.

The EpiMatrix is a T-cell epitope mapping algorithm which screens protein sequences for 9 to 10 amino acid long peptide segments predicted to bind to one or more MHC alleles (see e.g. De Groot, AS, Jesdale, BM, Szu, E, Schafer, JR. An interactive web site providing MHC ligand predictions: application to HIV research. AIDS Res. and Human Retroviruses. 1997; 13: 539-541; and Schafer J A, Jesdale B M, George J A, Kouttab N M, De Groot, AS. Prediction of well-conserved HIV-1 ligands using a Matrix-based Algorithm, EpiMatrix. Vaccine. 1998; 16(19):1880-1884.). EpiMatrix uses the pocket profile method for epitope prediction, which was first described by Sturniolo and Hammer in 1999. For reasons of efficiency and simplicity, predictions are limited to the eight most common HLA class II alleles and six “supertype” HLA class I alleles. EpiMatrix raw scores are normalized with respect to a score distribution derived from a very large set of randomly generated peptide sequences. Any peptide scoring above 1.64 on the EpiMatrix “Z” scale (approximately the top 5% of any given peptide set) has a significant chance of binding to the MHC molecule for which it was predicted. Peptides scoring above 2.32 on the scale (the top 1%) are extremely likely to bind; the scores of most well known T-cell epitopes fall within this range of scores. The EpiMatrix has been made publicly available, e.g. through the iVAX Toolkit (see e.g. the iVAX website, www.http://i-cubed.org/tools/ivax/ivax-tool-kit/) on the i-cubes website, where also further information is available. The identification of tregitopes is exemplified on page 36, line 4 to page 44, line 30 and in Examples 1, 2 and 3 of WO 2008/094538 A2, which are incorporated herein by reference; see also page 14, lines 8 to 23 of WO 2008/094538 A2.

The main functional characteristics of tregitopes in the sense of the present application are:
(i) tregitopes are presented by antigen-presenting cells like dendritic cells.
(ii) tregitopes bind to the MHC II pocket of the HLA complex of said antigen-presenting cells.
(iii) Presentation of tregitopes leads to activation of regulatory T-cells.
(iv) Presentation of tregitopes does not activate effective T cells.

Although the processes and mechanisms effected by tregitopes are complex, it is possible to further confirm the nature of a peptide to be a tregitope by suitable assays, e.g. the so-called TT (Tetanus Toxoid) assay as described in the examples section below. The assay is based on a tregitope-mediated suppression of CD4 T cell recall response in PBMC using tetanus toxoid as an antigen.

Examples for suitable tregitopes are provided below. Additional tregitopes are disclosed in Table 2 of WO2008/094538 A2 and in the sequences disclosed in WO 2016/054114 A1, which could also be used in the context of the present invention. Examples for suitable tregitopes include:

| SEQ ID NO: 10 | |
|---|---|
| (Treg289): | EEQYQSTYRVVSVLTVLHQDW, |
| SEQ ID NO: 7 | |
| (Treg084): | GTDFTLTISSLQPED, |
| SEQ ID NO: 2 | |
| (Treg009A): | GGLVQPGGSLRLSCAASGFTF, |
| SEQ ID NO: 9 | |
| (Treg088x): | KTLYLQMNSLRAEDTAKHYCA, |
| SEQ ID NO: 8 | |
| (Treg134): | LNNFYPREAKVQWKVDNALQSGNS, |
| SEQ ID NO: 3 | |
| (Treg029B): | MHWVRQAPGKGLEWV, |
| SEQ ID NO: 4 | |
| (Treg088): | NTLYLQMNSLRAEDTAVYYCA, |
| SEQ ID NO: 5 | |
| (Treg167): | PAVLQSSGLYSLSSVVTVPSSSLGTQ, |
| SEQ ID NO: 6 | |
| (Treg289n-native): | EEQYNSTYRVVSVLTVLHQDW. |

There may be minor modifications within the sequence of naturally occurring tregitopes. For example, it may be advantageous to include modifications, especially substitutions of single amino acids, in order to alter and especially reduce the hydrophobicity of the tregitopes by incorporation of amino acids which are charged at physiological pH. Table 2 of WO2008/094538 provides examples regarding possible variation of tregitopes. Two preferred examples of modified sequences of naturally occurring tregitopes are Treg088x and Treg289, wherein the modifications within the sequences are underlined:

| SEQ ID NO: 9 | |
|---|---|
| (Treg088x): | KTLYLQMNSLRAEDTAKHYCA |
| SEQ ID NO: 10 | |
| (Treg289): | EEQYQSTYRVVSVLTVLHQDW. |

Moreover, trimmed sequences may be used, i.e. one or more amino acids at the ends of the sequences representing the tregitopes, especially two or three amino acids at the ends, may be omitted while maintaining the function of the T cell binding epitope. Generally, a nine amino acid core motive of the tregitopes is important for presentation of the peptide during their natural immunologic processing. Preferably, said core sequence is present in the sequences of the preferred tregitopes. In the following, some preferred trimmed sequences of tregitopes are shown:

SEQ ID NO: 11

| | |
|---|---|
| (trimmed Treg009A): | VQPGGSLRLSCAASG, |

SEQ ID NO: 12

| | |
|---|---|
| (trimmed Treg029B-v1): | WVRQAPGKGL, |

SEQ ID NO: 13

| | |
|---|---|
| (trimmed Treg029B-v2): | VRQAPGKGL, |

SEQ ID NO: 14

| | |
|---|---|
| (trimmed Treg088): | YLQMNSLRAEDTAVY, |

SEQ ID NO: 15

| | |
|---|---|
| (trimmed Treg088x-v1): | KTLYLQMNSLRAEDTAKH, |

SEQ ID NO: 16

| | |
|---|---|
| (trimmed Treg088x-v2): | YLQMNSLRAEDTAKH, |

SEQ ID NO: 17

| | |
|---|---|
| (trimmed Treg167): | LQSSGLYSLSSVVTVPSSSL, |

SEQ ID NO: 18

| | |
|---|---|
| (trimmed Treg289n): | YNSTYRVVSVLTVLH, |

SEQ ID NO: 19

| | |
|---|---|
| (trimmed Treg289): | YQSTYRVVSVLTVLH, |

SEQ ID NO: 20

| | |
|---|---|
| (trimmed Treg084): | FTLTISSLQ, |
| and | |

SEQ ID NO: 21

| | |
|---|---|
| (trimmed Treg134): | FYPREAKVQWKVDNALQS. |

Treg289, Treg084, Treg009A, Treg088x and Treg134 (both not trimmed and trimmed versions) have shown particularly good results in expression in the context of the TCPs according to the present invention.

Optionally, in the TCP of the invention, two, three or all tregitopes of the TCP are tregitopes of SEQ ID NO: 2-21, preferably, of SEQ ID NO: 2, 7, 8, 9, 10, 11, 15, 16, 19 and 20.

Preferably, all heterologous tregitopes in one TCP chain may have different sequences. Using different tregitopes improves the potential to target and activate regulatory T cells of subjects with different HLA haplotypes and different recognition, processing or presentation capabilities. Alternatively, some or all heterologous tregitopes in one TCP monomer have the same sequence, e.g., targeted to presentation on a suitable HLA haplotype or set of haplotypes.

Heterologous Tregitopes

The term “heterologous tregitope” in the context of the present invention means that the tregitope does not occur identically in the same position in the respective immunoglobulin Fc-part chain. Thus, more particularly, the term “heterologous tregitope” means that the tregitope
(i) does not naturally occur in an immunoglobulin Fc-part chain, and/or
(ii) is not located at its natural position in the immunoglobulin Fc-part chain.

The term “not naturally occurring” in this context comprises the case that the tregitope sequence is similar to a naturally occurring tregitope, but has one or more modifications that differentiates it from any tregitope present in a corresponding Fc-part of an unmodified natural antibody, in particular a natural human IgG antibody. Such modification may be, e.g., a deletion, insertion, inversion or substitution, preferably, a substitution.

In context with SEQ ID NO: 1, the term “heterologous” in the context of the present invention means that the tregitope does not occur identically in the same position in the Fc-part chain according to SEQ ID NO: 1, more particularly not in the amino acid sequence from position 135 to position 330 of SEQ ID NO: 1. Preferably, it also does not occur identically in the same position in a sequence having at least 85% sequence identity to SEQ ID NO: 1, e.g., a naturally occurring sequence.

It is noteworthy that in human IgG Fc-part chains, more particularly in said immunoglobulin Fc-part chain according to amino acids No. 135-330 of SEQ ID NO: 1, there is one naturally occurring tregitope. This is tregitope 289 (SEQ ID NO: 10), which is, in wildtype IgG of SEQ ID NO: 1, located in sequence frame A. If it is located in another position, e.g., in sequence frame B or C, it is considered a heterologous tregitope. Also mentioned in this specification is a sequence variant of tregitope 289 (tregitope 289x), which is also considered a heterologous tregitope for the purpose of the present invention, as it differs from the naturally occurring tregitope. This also applies if said tregitope is located in the same position as tregitope 289 in SEQ ID NO: 1. The tregitopes of SEQ ID NO: 2-9 and 11-21 are thus heterologous tregitopes regardless of their position in SEQ ID NO: 1.

In addition to the at least one heterologous tregitope, the TCP of the invention may further comprise at least one “homologous” tregitope, i.e. a tregitope naturally occurring in an Fc-part chain of SEQ ID NO: 1 or having at least 85% sequence identity thereto, such as tregitope 289 naturally occurring in the Fc-part chain according to SEQ ID NO: 1.

For example, if the TCP of the invention comprises at least one heterologous tregitope in frame B or C, which is preferred in the context of the invention, the TCP may further comprise a homologous tregitope in frame A, in particular, tregitope 289. Accordingly, such a TCP comprises at least two tregitopes, or, if there is a heterologous tregitope in each of frames B and C, at least three tregitopes.

Preferably, the TCP of the present invention comprises at least two heterologous tregitopes, more preferably at least three, optionally, four heterologous tregitopes. Optionally, the TCP comprises two to four tregitopes.

Integration of Tregitopes

In principle, the skilled person may choose the location in the TCP or in the Fc-part chain of an immunoglobulin where the tregitope(s) should be integrated as deemed appropriate. However, preferred positions are outside of parts of the TCP which are responsible for formation of the tertiary or quaternary structure of the resulting protein. Parts of the TCP which are responsible for formation of a tertiary or quaternary structure comparable to the structure of an Fc part of an immunoglobulin may e.g. be amino acids like cysteines which form disulfide bonds, or amino acids responsible for glycosylation. Thus, in preferred embodiments, the sequences representing tregitopes are located in the TCP in such a way that intra-molecular disulfide bonds stabilizing the tertiary structure are maintained and/or that glycosylation is maintained. In certain embodiments, it may also be desired to maintain the hinge region or parts thereof, in order to allow for dimerization. In this case, the tregitopes should be located in the TCP in such a way that inter-molecular disulfide bonds stabilizing the quaternary structure are maintained.

Particularly advantageous frames, regions suitable for integration of tregitopes, that have been identified in the context of the present invention are described in detail herein.

In particular, for TCP derived from IgG, such as IgG1, the inventors found that it is advantageous e.g., for expression and stability of the TCP, if the one or more heterologous tregitopes is/are located within sequence frames A, B, or C, wherein
(a) sequence frame A corresponds to positions 168 to 203 of SEQ ID NO: 1,
(b) sequence frame B corresponds to positions 272 to 307 of SEQ ID NO: 1, and
(c) sequence frame C corresponds to positions 212 to 249 of SEQ ID NO: 1.

Preferably, (a) sequence frame A corresponds to positions 170 to 203 of SEQ ID NO: 1, (b) sequence frame B corresponds to positions 275 to 306 of SEQ ID NO: 1, and (c) sequence frame C corresponds to positions 214 to 249 of SEQ ID NO: 1.

Optionally, (a) sequence frame A corresponds to positions 173 to 203 of SEQ ID NO: 1, (b) sequence frame B corresponds to positions 277 to 304 of SEQ ID NO: 1, and (c) sequence frame C corresponds to positions 217 to 248 of SEQ ID NO: 1.

Each frame allows integrating a tregitope in the TCP, whereas expression of the TCP is still possible in an acceptable manner. More particularly, the tertiary structure of the Fc-part chain is not affected in an inacceptable manner. For example, advantageously, the intramolecular disulfide bonds stabilizing the CH2 and CH3 domains are maintained. Also, dimer formation is possible, if desired. The definition of these frames by the inventors yields a very flexible platform for integrating tregitopes. Frames B and C have been particularly difficult to identify. The skilled person will understand that the TCP sequence outside of the inserted tregitopes may also be subject to certain variation without fundamentally impairing the substantial advantages of the invention. For example, allelic variants of SEQ ID NO: 1, i.e., other Fc-part chains, may be used. The skilled person is aware of many variants of Fc-part chains, for example mammalian Fc-part chains or human and non-human Fc-part chains. For human therapeutic applications, a human Fc-part chain, such as an Fc-part chain from human IgG, IgA, or IgM is preferred, preferably human IgG1, IgG2, IgG3 or IgG4, more preferably IgG1 and IgG4. Most preferred are human IgG1 Fc-part chains.

A TCP of the invention may be derived from immunoglobulins other than IgG, e.g., from IgA, IgM, IgE or IgD, preferably, from the CH2 and CH3 domains thereof, wherein the TCP may optionally comprise further constant domains, in particular, a CH4 domain, and/or further regions, such as a joining chain, if typically present in said immunoglobulin. For such TCPs, appropriate frames for integration of at least one heterologous tregitope may also be identified in positions of the Fc-part chain that show a comparatively high percentage of sequence similarities with the sequences of the respective tregitopes, e.g., a sequence similarity of at least 85%, at least 90% or at least 95%. This may be analyzed by a sequence alignment as described in the experimental part (see e.g. FIGS. 4, 5, and 6). In this context “similarities” means that the aligned amino acids are identical or show similar characteristics, e.g., polar amino acids may be exchanged against other polar amino acids, unpolar amino acids may be exchanged against other unpolar amino acids, basic amino acids may be exchanged against other basic amino acids or acidic amino acids may be exchanged against other acidic amino acids without detracting from the similarity of the sequence. In one embodiment, said similarity is sequence identity. Such TCP typically show an overall sequence identity to constant domains (in particular, the CH2 and CH3 domain) of the IgA, IgM, IgE or IgD from which they are derived of at least 50%, at least 60%, at least 70%, at least 80%, at least 85%, or at least 90%. Optionally, if the IgA, IgM, IgE or IgD from which such TCP are derived comprise one or more homologous tregitope(s), said homologous tregitope may be exchanged against a heterologous tregitope. However, preferably, such TCP comprise at least one heterologous tregitope in a different position. The positon of frames A, B or C in the TCP of the present invention derived from IgG may serve as guidance in identifying suitable frames in TCP derived from other Ig. for example, tregitopes may be incorporated in corresponding positions. Amino acid positions important for intramolecular disulfide bonds are typically maintained. Also, glycosylation of the Fc parts may maintained, but it may also be absent. It has been shown that, for Ig other than IgG, glycosylation is not required for some effector functions such as receptor binding. In certain embodiments, it may also be desired to maintain the hinge region of the respective Ig or parts thereof, in order to allow for dimerization. In this case, the tregitopes should be located in the TCP in such a way that inter-molecular disulfide bonds stabilizing the quaternary structure are also maintained.

The TCP into which the tregitopes are integrated may also comprise further modifications deemed appropriate or useful, such as truncations, additions, deletions, insertions, inversions, or substitutions. Such modifications may, e.g., serve to eliminate or promote formation of disulfide bridges (e.g. via cysteine residues in the hinge region), as desired. Other modifications may be introduced to improve or reduce binding to Fc-receptors as may be desired depending on the intended purpose. Preferably, the modifications should not impair the manufacture of the TCP (such as recombinant expression and/or secretion). Further modifications are contemplated such as glycosylation, phosphorylation, PEGylation or HESylation. For example, PEGylation may be useful to further increase the half-life of the TCP. The skilled person knows how to introduce such modifications.

However, although the skilled person may accept a certain negative impact of sequence modifications on the expression level, any sequence modifications should be chosen such that they do not affect the expression of the TCP in a non-acceptable manner. Preferably, the expression level should not be lower than 5%, 10%, 20%, 50% or preferably, it should be at least 80% of the expression level of a polypeptide of SEQ ID NO: 54, construct V32, if expressed under the same conditions, preferably, as described in the examples below.

In the TCP of the present invention, it is preferred that,
(a) if sequence frame A contains no heterologous tregitope, said frame A has at least 85% sequence identity with positions 168 to 203 of SEQ ID NO: 1, and
(b) if sequence frame B contains no heterologous tregitope, said frame B has at least 85% sequence identity with positions 272 to 307 of SEQ ID NO: 1, and
(c) if sequence frame C contains no heterologous tregitope, said frame C has at least 85% sequence identity with positions 212 to 249 of SEQ ID NO: 1. Of course, the sequence identity with the respective positions in those frames that do not contain a heterologous tregitope may also be higher, preferably, at least 90%, optionally, at least 99% or 100%.

If a heterologous tregitope is stated to be “located within” a sequence frame, this means that the whole sequence of said heterologous tregitope is integrated into the corresponding sequence frame, e.g. by substitution, or partial substitution of the respective wildtype sequence. Preferably, in the TCP of the present invention, the at least one heterologous tregitope (or all heterologous tregitopes present in frames A, B or C) substitutes a sequence within the regions spanning amino acids 135 to 330 of SEQ ID NO: 1 having the same length as said tregitope or having the length of the tregitope plus or minus one or two amino acids. Disruptions of the tertiary and quaternary structure are typically minimized if the heterologous tregitope substitutes a sequence having the same length as said tregitope. The skilled person may consider to introduce further sequence changes in the sequence frame as deemed appropriate. However, typically the parts of the sequence frames not substituted by the heterologous tregitope(s) do not need to be changed further. Accordingly, they preferably have at least 85%, at least 90%, at least 95%, at least 99% or 100% sequence identity with the respective positions of SEQ ID NO: 1.

Alternatively, or, preferably, in addition to the heterologous tregitopes located within one or more of frames A, B and C, the TCP of the invention may also comprise a tregitope C-terminal to the amino acid sequence having at least 85% sequence identity with amino acids 135 to 330 of SEQ ID NO: 1. Said C-terminal tregitope is either directly C-terminal to the amino acid sequence having at least 85% sequence identity with amino acids 135 to 330 of SEQ ID NO: 1, or linked to said sequence via a linker, e.g., a linker of less than 18 amino acids, optionally, less than 12 amino acids or less than 5 amino acids. Preferably, a linker of 3-18 amino acids is employed.

Said C-terminal heterologous tregitope may be at the C-Terminus of the TCP, optionally, linked to said sequence via a linker of 3-18 amino acids. Alternatively, the heterologous tregitope C-terminal to the amino acid sequence having at least 85% sequence identity with amino acids 135 to 330 of SEQ ID NO: 1 is not at the C-terminus of the TCP, in this case, preferably, the TCP is a fusion protein.

The linker may be a GS linker, e.g., as known in the art. For example, a linker like (GGSG)ₙ (SEQ ID NO: 110) may be used, wherein “n” means one or more (e.g., 2, 3 or 4) repeats of said sequence.

Further preferred examples of linkers are:

| (SEQ ID NO: 107) | |
|---|---|
| Linker 1: | Ala Gly Pro Gly Pro Ser Gly |
| (SEQ ID NO: 108) | |
| Linker 2: | Pro Thr Gly Ser Gly |
| (SEQ ID NO: 109) | |
| Linker 3: | Gly Gly Ser Thr Gly |

The inventors could show that use of linker 2 particularly improves binding energy of the resulting TCP dimers, i.e., stability of the protein.

Preferred tregitopes for inclusion C-terminal to the amino acid sequence having at least 85% sequence identity with amino acids 135 to 330 of SEQ ID NO: 1 are Treg134, Treg088x and Treg088.

As shown in the examples below, the heterologous tregitope C-terminal to the amino acid sequence having at least 85% sequence identity with amino acids 135 to 330 of SEQ ID NO: 1 may also be Treg029B, in particular, for use with a linker such as linker 2 (SEQ ID NO: 108).

It is also contemplated that one or more tregitopes may be added N-terminally of the amino acid sequence having at least 85% sequence identity with amino acids 135 to 330 of SEQ ID NO: 1, preferably, at the N-terminus of the protein.

The invention provides a TCP, wherein a first heterologous tregitope is located in one of frames A, B, or C, and wherein at least a second tregitope is located in a different frame of frames A, B, C, or C-terminal to the amino acid sequence having at least 85% sequence identity with amino acids 135 to 330 of SEQ ID NO: 1, optionally linked to said sequence via a linker, e.g., of 3-18 amino acids.

For example, if the TCP contains two heterologous tregitopes, these may be located in frames A and B, frames A and C or, preferably, in frames B and C. In that case, it is preferred that frame A comprises the homologous tregitope Treg289 (positions 176-196 of SEQ ID NO: 1). The heterologous tregitopes may also be located in frame A and C-terminal to the amino acid sequence having at least 85% sequence identity with amino acids 135 to 330 of SEQ ID NO: 1, or in frame B and C-terminal to the amino acid sequence having at least 85% sequence identity with amino acids 135 to 330 of SEQ ID NO: 1, or in frame C and C-terminal to the amino acid sequence having at least 85% sequence identity with amino acids 135 to 330 of SEQ ID NO: 1.

If the TCP comprises three heterologous tregitopes, these may be located in frames A, B and C, or in frames A, B and C-terminal to the amino acid sequence having at least 85% sequence identity with amino acids 135 to 330 of SEQ ID NO: 1, or in frames A, C and C-terminal to the amino acid sequence having at least 85% sequence identity with amino acids 135 to 330 of SEQ ID NO: 1, or in frames B, C and C-terminal to the amino acid sequence having at least 85% sequence identity with amino acids 135 to 330 of SEQ ID NO: 1. If there are four heterologous tregitopes, these may be located in frames A, B and C, and C-terminal to the amino acid sequence having at least 85% sequence identity with amino acids 135 to 330 of SEQ ID NO: 1.

Preferred positions for certain preferred tregitopes are provided in the following tables, with the numbering of positions referring to SEQ ID NO: 1. Corresponding advantageous positions in other Fc-part chains can be found by sequence alignment as known to the skilled person.

Preferred positions of tregitopes in frame A

| | Position in |
|---|---|
| Tregitope | SEQ ID NO: 1 |
| Treg289 | 176 to 196 |
| Treg167 | 174 to 199 |
| Treg009A | 180 to 200 |
| Treg029B | 178 to 192 |
| Treg084 | 186 to 200 |
| Treg134 | 179 to 202 |
| Treg088x-v1 | 173 to 190 |

Preferred positions of tregitopes in frame B

| | Position in |
|---|---|
| Tregitope | SEQ ID NO: 1 |
| Treg289 | 280 to 300 |
| Treg167 | 278 to 303 |
| Treg009A | 278 to 298 |
| Treg029B | 287 to 301 |
| Treg084 | 284 to 298 |
| Treg134 | 277 to 300 |
| Treg088x-v1 | 287 to 304 |

Preferred positions of tregitopes in frame C

| | Position in |
|---|---|
| Tregitope | SEQ ID NO: 1 |
| Treg289 | 225 to 245 |
| Treg167 | 223 to 248 |
| Treg009A | 223 to 243 |
| Treg029B | 223 to 237 |
| Treg084 | 224 to 238 |
| Treg134 | 222 to 245 |
| Treg088x-v1 | 217 to 234 |

Preferred TCP

The inventors have identified particularly advantageous positions for specific tregitopes in specific frames. In preferred embodiments, in the TCP of the invention,
(a) Frame A comprises the tregitope of SEQ ID NO: 10 (Treg289) at position 176 to 196 (i.e., at the position corresponding to the respective position of SEQ ID NO: 1), SEQ ID NO: 5 (Treg167) at position 174 to 199, SEQ ID NO: 2 (Treg009A) at position 180 to 200, SEQ ID NO: 3 (Treg029B) at position 178 to 192, SEQ ID NO: 7 (Treg084) at position 186 to 200, SEQ ID NO: 8 (Treg134) at position 179 to 202, or SEQ ID NO: 15 (trimmed Treg088x-v1) at position 173 to 190; and/or
(b) frame B comprises the tregitope of SEQ ID NO: 10 (Treg289) at position 280 to 300, SEQ ID NO: 5 (Treg167) at position 278 to 303, SEQ ID NO: 2 (Treg009A) at position 278 to 298, SEQ ID NO: 3 (Treg029B) at position 287 to 301, SEQ ID NO: 7 (Treg084) at position 284 to 298, SEQ ID NO: 8 (Treg134) at position 277 to 300, or SEQ ID NO: 15 (trimmed Treg088x-v1) at position 287 to 304; and/or
(c) frame C comprises the tregitope of SEQ ID NO: 10 (Treg289) at position 225 to 245 (or at the position corresponding to the respective position of SEQ ID NO: 1), SEQ ID NO: 5 (Treg167) at position 223 to 248, SEQ ID NO: 2 (Treg009A) at position 223 to 243, SEQ ID NO: 3 (Treg029B) at position 223 to 237, SEQ ID NO: 7 (Treg084) at position 224 to 238, SEQ ID NO: 8 (Treg134) at position 222 to 245, or SEQ ID NO: 15 (trimmed Treg088x-v1) at position 217 to 234; and/or
(d) at least one heterologous tregitope located C-terminal to the amino acid sequence having at least 85% sequence identity with amino acids 135 to 330 of SEQ ID NO: 1 has SEQ ID NO: 8 (Treg134), SEQ ID NO: 14 (trimmed Treg088) or SEQ ID NO: 9 (Treg088x), wherein said tregitope is optionally linked to said sequence having at least 85% sequence identity with amino acids 135 to 330 of SEQ ID NO: 1 via a linker of 3-18 amino acids such as a GS linker or a linker of any of SEQ ID NO: 107-110.

Examples for TCP of the invention with suitable combinations of specific tregitopes include:
(I) a TCP comprising
   (a) a tregitope according to SEQ ID NO: 2 (Treg009A) located in frame A,
   (b) a tregitope according to SEQ ID NO: 2 (Treg009A) located in frame B,
   (c) a tregitope according to SEQ ID NO: 7 (Treg084) located in frame C, and
   (d) a tregitope according to SEQ ID NO: 9 (Treg088x) located C-terminal to the amino acid sequence having at least 85% sequence identity with amino acids 135 to 330 of SEQ ID NO: 1, optionally via a linker of 3-18 amino acids such as a GS linker (V32);
(II) a TCP comprising
   (a) a tregitope according to SEQ ID NO: 9 (Treg088x) located in frame B, and
   (b) a tregitope according to SEQ ID NO: 2 (Treg009A) located in frame C (V20);
(III) a TCP comprising
   (a) a tregitope according to SEQ ID NO: 10 (Treg289) located in frame B, and
   (b) a tregitope according to SEQ ID NO: 9 (Treg088x) located C-terminal to the amino acid sequence having at least 85% sequence identity with amino acids 135 to 330 of SEQ ID NO: 1, optionally via a linker of 3-18 amino acids such as a GS linker (V34);
(IV) a TCP comprising
   (a) a tregitope according to SEQ ID NO: 10 (Treg289) located in frame A, and
   (b) a tregitope according to SEQ ID NO: 7 (Treg084) located in frame C, and
   (c) a tregitope according to SEQ ID NO: 8 (Treg134) located C-terminal to the amino acid sequence having at least 85% sequence identity with amino acids 135 to 330 of SEQ ID NO: 1, optionally via a linker of 3-18 amino acids such as a GS linker (V1);
(V) a TCP comprising
   (a) a tregitope according to SEQ ID NO: 10 (Treg289) located in frame A,
   (b) a tregitope according to SEQ ID NO: 8 (Treg134) located in frame B, and
   (c) a tregitope according to SEQ ID NO: 7 (Treg084) located in frame C (V3);
(VI) a TCP comprising
   (a) a tregitope according to SEQ ID NO: 10 (Treg289) located in frame A, and
   (b) a tregitope according to SEQ ID NO: 7 (Treg084) located in frame C, and
   (c) a tregitope according to SEQ ID NO: 9 (Treg088x) located C-terminal to the amino acid sequence having at least 85% sequence identity with amino acids 135 to 330 of SEQ ID NO: 1, optionally via a linker of 3-18 amino acids such as a GS linker (V13);
(VII) a TCP comprising
   (a) a tregitope according to SEQ ID NO: 7 (Treg084) located in frame C, and
   (b) a tregitope according to SEQ ID NO: 8 (Treg134) located C-terminal to the amino acid sequence having at least 85% sequence identity with amino acids 135 to 330 of SEQ ID NO: 1, optionally via a linker of 3-18 amino acids such as a GS linker (V14).

In the context of the invention, the inventors have provided specific TCP comprising an amino acid sequence of SEQ ID NOs: 23 to 44 (V1-V22) and 46 to 58 (V24-V36). V32, V20, V34, V1, V3, V13 and V14 show a particularly high expression and are thus preferred TCP of the invention. V32 is the construct comprising the most tregitopes.

An alternative to V32 of SEQ ID NO: 54 is V32_variant of SEQ ID NO: 111, which has a deletion of amino acid R238 according to SEQ ID NO: 1 in frame C.

Different TCP Formats

The TCP of the invention may be used in different formats.
TCP as a stand-alone agent

For example, it may be used as a stand-alone agent, e.g., a stand-alone therapeutic agent, wherein the TCP is not linked to other agents or moieties, in particular, wherein it is not expressed as a fusion protein with other, e.g., therapeutic polypeptides. In this context, the TPC may be used either as a monomer or as a multimer, e.g., a dimer.

Accordingly, the invention provides a TCP comprising from 195 to 350 amino acids. The invention also provides a TCP essentially consisting of the amino acid sequence having at least 85% sequence identity with amino acids 135 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity, wherein said TCP optionally further comprises a tregitope C terminal to the amino acid sequence having at least 85% sequence identity with amino acids 135 to 330 of SEQ ID NO: 1, which may be linked to said sequence with a linker, e.g., consisting of 3-18 amino acids. Said TCP may consist of 195 to 350 amino acids, preferably, 200-330 amino acids, e.g., 205-300 amino acids, 210 to 251 amino acids or 220-230 amino acids. Preferred TCP that may be used in this format are disclosed herein, e.g., above.

In this context, “essentially consisting” does not exclude the presence of additional sequences having at least 85% sequence identity to positions 99-330 of SEQ ID NO: 1, in particular, presence of the sequences corresponding to the CH2 and CH3 regions (with integrated tregitopes) and, optionally, the hinge region. The TCP may also comprise a signal sequence. However, preferably, said TCP does not comprise the VH domain and/or the CH1 domain of an antibody.

In certain further embodiments, the TCP consists of or essentially consists of a polypeptide sequence having at least 60%, preferably at least 70% sequence identity to amino acids 135 to 330 SEQ ID NO: 1, wherein said TCP optionally further comprises a tregitope C terminal to the amino acid sequence having at least 85% sequence identity with amino acids 135 to 330 of SEQ ID NO: 1, which may be linked to said sequence with a linker, e.g., consisting of 3-18 amino acids.

In certain further embodiments, the TCP consists of or essentially consists of a polypeptide sequence having at least 70%, preferably at least 80% sequence identity to amino acids amino acids 99 to 330 of SEQ ID NO: 01, wherein said TCP optionally further comprises a tregitope C terminal to the amino acid sequence having at least 85% sequence identity with amino acids 135 to 330 of SEQ ID NO: 1, which may be linked to said sequence with a linker, e.g., consisting of 3-18 amino acids.

In certain further embodiments, the TCP consists of or essentially consists of a polypeptide sequence having at least 70%, preferably at least 80% sequence identity to amino acids amino acids 80 to 330 of SEQ ID NO: 01, wherein said TCP optionally further comprises a tregitope C terminal to the amino acid sequence having at least 85% sequence identity with amino acids 135 to 330 of SEQ ID NO: 1, which may be linked to said sequence with a linker, e.g., consisting of 3-18 amino acids.

In this context “essentially consisting of” also means that the TCP may comprise additional components like an affinity tag for purification, but generally the TCP in this context does not comprise a fused protein or peptide which by itself has a therapeutic or physiologic effect like an allergen.

Preferably, the TCP according to the present invention does not comprise amino acid sequences of more than 100, preferably of more than 50, more preferably more than 20 contiguous amino acids having less than 50%, more particularly less than 75%, 85%, 90% sequence identity to positions 99-330 of SEQ ID NO: 1 or a tregitope sequence, more particularly a tregitope sequence as disclosed herein.

Such a TCP may be a monomer, wherein the TCP typically does not comprise a part that enables dimer formation, i.e., it does not comprise the hinge region of an immunoglobulin or a part thereof that enables dimerization. Further, monomers may also be modified in the CH3 domain to reduce dimerisation, e.g., by introducing a K292R substitution, wherein the position refers to SEQ ID NO: 1.

Alternatively, such a TCP may be a multimer, e.g., a dimer. The TCP may optionally be modified to increase multimerisation, e.g., dimer formation, e.g., in the CH3 domain, The TCP may also be a trimer, a tetramer, a pentamer or a hexamer. Multimers are further characterized below. Preferably, a TCP in a stand-alone format forms a multimer, in particular, a dimer.

Thus, in certain embodiments, the TCP according to the present invention essentially corresponds to or consists of the monomer, dimer, or multimer of an Fc-part chain comprising one or more heterologous tregitopes, preferably located in the sequence frames described and/or C-terminal of the amino acid sequence having at least 85% sequence identity to positions 135-330 of SEQ ID NO: 1, wherein a C-terminal tregitope may be linked to said sequence with a linker, e.g., consisting of 3-18 amino acids.
TCP comprising further antibody domains

The TCP according to the present invention may comprise further peptide or polypeptide sequences apart from sequences corresponding to Fc-part chain and tregitope sequences. The invention also provides a TCP according to the invention, wherein the TCP comprises further immunoglobulin superfamily domains.

For example, the TCP of the invention may comprise at least a VH domain and CH1 domain of an antibody, preferably, an antigen-binding part of an antibody (typically, IgG). Different structures of antigen binding parts of an antibody are known in the art, e.g., the TCP of the invention can comprise a VH domain and CH1 domain and be associated with a light chain with a VL and CL domain, wherein the VH and CH domains form the antigen-binding site of the antigen-binding part. Alternatively, the antigen-binding domain may be a scFv, wherein, preferably, the scFv is expressed as a fusion protein with the TCP.

Alternatively or additionally, said TCP may further comprise a CH3 domain of IgA, and, optionally, a joining region of IgA, which allows for formation of a tetrameric protein including 4 TCP monomers. For example, said TCP may further comprise a CH3 and CH4 domain of IgM, which allows for formation of a multimer with 10 TCP monomers.
TCP linked to further agents

The TCP according to the invention may be linked to one or more further agents. Such agents may have a non-therapeutic function, e.g., to increase or facilitate expression or purification. For example, the TCP may further comprises an affinity tag, e.g., albumin, an albumin-binding domain or a His-tag. The TCP according to the invention may also further comprise a linker, e.g., a GS linker or a linker of any of SEQ ID NO: 107-110.

The TCP according to the invention may additionally or alternatively be linked to one or more agents having a therapeutic or preventive function.

The TCP of the invention may e.g., be covalently or non-covalently linked to an agent, wherein the agent preferably is an agent against which an undesired immune reaction is to be suppressed and/or immunogenic tolerance is to be conferred. “Suppression of an immune response”, in the context of the invention means that an immune response is reduced or completely abrogated. This also includes the case that the nature of the immune response is changed in a way that avoids or reduces undesired effects of the immune response, e.g., inflammation and/or formation of antibodies. Further, an immune response can also be prevented by suppression of an immune response. Such suppression of an immune response and/or induction of immunogenic tolerance may be mediated by activation of regulatory T cells.

Preferably, the TCP is covalently linked to the agent of interest. It can be linked as a monomer, or multimer (e.g. dimer). The TCP is particularly easy to link to other agents. As a polypeptide, the TCP can be linked in a particularly easy way to other proteins or peptides by recombinant techniques, resulting in a TCP fusion protein. Thus, the present invention also relates to a TCP fusion protein comprising the TCP and an agent against which an undesired immune reaction shall be suppressed and/or immunogenic tolerance is to be conferred.

In a fusion protein, the agent is preferably coupled N-terminally, e.g., in place of the CH1 domain or the hinge region, if absent in said TCP (wherein, typically, if dimerisation is intended, the hinge region is present, and if dimerisation is not intended, the hinge region is absent). Fusion proteins may be linked via a linker, e.g., a GS linker or a linker of any of SEQ ID NO: 107-110.

If the hinge region is included in the TCP, the TCP may also be linked to the agent via one or more disulfide bridges. Chemical coupling, e.g., to lysine residues in the TCP is also possible. Alternatively, the TCP may be non-covalently linked to said agent, e.g., associated with the agent via van-der-Waals interactions, ionic interactions or hydrophobic interactions, polar interactions (dipol, quadrupole, or higher), and aromatic interactions (quadrupole/quadrupole or π/π). However, preferably the binding is sufficiently stable under physiological conditions to maintain close association of the TCP and the agent.

Linking of the TCP to peptides or polypeptides is particularly easy, e.g. by recombinant means and methods. Furthermore, peptides and polypeptides play a role in many undesired immune reactions, such as autoantigens or foreign antigens. Therefore, in certain preferred embodiments, the agent is a peptide or polypeptide moiety.

An “undesired immune reaction” may, e.g., be an allergy, autoimmunity or an immune reaction against a transplant, e.g., a graft rejection reaction. An undesired immune reaction may be mainly mediated by antibodies or by cellular mechanisms such as cytotoxic T cells. It may e.g., be a T_{H}1 or a T_{H}2 response.

Said agent may be, e.g., (a) an allergen, (b) an intolerance inducing agent, (c) a target protein of an autoimmune response, e.g., of an autoantibody, (d) a target epitope of an autoimmune response, e.g., of an autoantibody, or (e) a therapeutic agent.

The term “allergen” is generally known to the skilled person. An allergen is a non-self agent which has the capacity to cause an undesired or abnormally vigorous immune reaction in a subject exposed to the allergen. More specifically, an allergen is an antigen capable of stimulating a type-I hypersensitivity reaction in atopic individuals through Immunoglobulin E (IgE) responses.

Examples of allergens include:
(a) food allergens such as celery and celeriac, corn or maize, eggs (typically albumen, the white), legumes (e.g. beans, peas, peanuts, soybeans), milk, seafood, sesame, soy, tree nuts (e.g. pecans, almonds)
(b) insect venoms (e.g. bee venom, wasp venom, mosquito venom)
(c) plant pollens (hay fever), such as grass pollen (e.g. ryegrass, timothy-grass), weed pollen (e.g. ragweed, plantago, nettle, Artemisia vulgaris, Chenopodium album, sorrel), tree pollen (e.g. birch, alder, hazel, hornbeam, Aesculus, willow, poplar, Platanus, Tilia, Olea, Ashe juniper, Alstonia scholaris)
(d) drugs (e.g. penicillin, sulfonamides, salicylates (also found naturally in numerous fruits)
(e) animal products (e.g. Fel d 1 (Allergy to cats), fur and dander, cockroach calyx, wool, dust mite excretion.

In general, it may be sufficient just to use fragments comprising the relevant epitopes of such allergens. Thus, for example, the invention provides a TCP linked with epitopes, fragments or complete peptides/proteins of bee venom, a TCP linked with epitopes, fragments or complete peptides/proteins of wasp venom, a TCP linked with epitopes, fragments or complete peptides/proteins of mosquito venom or a TCP linked with epitopes, fragments or complete peptides/proteins of plant pollen.

An immunological “intolerance inducing agent” is a non-self agent capable of causing an immunological intolerance reaction, i.e. an undesired immunological response which is mediated by non-IgE immunoglobulins, in which the immune system recognises a particular agent as a foreign body. In contrast to an allergy, the response generally takes place over a prolonged period of time. Examples for intolerance inducing agents include: gluten, salicylates (the latter can also cause allergies). For example one or more components of gluten, such as proteins or peptides selected from alpha-/beta-, gamma- or omega-gliadines or their responsible epitopes may be considered in this context. Thus, the invention also provides TCP linked with epitopes, fragments or complete peptides/proteins of alpha-gliadines, and/or TCP linked with epitopes, fragments or complete peptides/proteins of beta-gliadines, and/or TCP linked with epitopes, fragments or complete peptides/proteins of gamma-gliadines, and/or TCP linked with epitopes, fragments or complete peptides/proteins of omega-gliadines, and/or TCP linked with salicylates.

“Target proteins” or “target epitopes” of an autoimmune response, e.g., of autoantibodies are known to the skilled person, e.g. from databases such as the AAgAtlas database, which allows to browse, retrieve and download a list of autoantigens and their associated diseases. This database is freely accessible at http://biokb.ncpsb.org/aagatlas. Fusion proteins or combinations of the TCP with auto-antigens may be suitably applied in rheumatic diseases, Hashimoto's thyroiditis, or IgG4-mediated autoimmune diseases. For example, a target protein may be tissue transglutaminase in the context of celiac disease, or insulin or insulin receptor or an islet cell antigen in the context of diabetes type I. Other target proteins may be Thyroid Stimulating Hormone Receptor (TSHR) or other Graves' disease antigens in the context of Graves' disease, or thyroid peroxidase and/or thyroglobulin TSHR in the context of autoimmune thyroiditis. A target epitope may, e.g., be an epitope from any of these target proteins, in particular, an epitope from said proteins presented on an MHC by the subject to be treated.

The term “therapeutic agent” comprises any drug, medicament, or other agent, which may be used to prevent or treat a disorder or disease, wherein said agent may be approved as a medicament. Quite a number of therapeutic agents are capable of eliciting undesired immune responses. Those responses can be allergic reactions (as mentioned above) or other undesired reactions. For example, many therapeutic agents are recognized by the immune system as foreign. This may lead to formation of anti-drug antibodies (also known as ADAs). Frequently, those antibodies are neutralizing, i.e. they block the therapeutic effect of the agent, either by blocking the agent from interacting with the intended therapeutic target (e.g. a certain receptor), or by accelerating the degradation of the therapeutic agent. This is of particular relevance in connection with certain substitution therapies, when the patient has a genetic lack of a certain endogenous protein or factor so that the immune system has not developed a tolerance to said endogenous protein or factor. Therefore, there is a strong need to convey immunological tolerance and/or to suppress undesired immune responses to such therapeutic agents. This is a particular problem for therapeutic protein or peptides, such as certain hormones, cytokines, enzymes, antibodies, coagulation factors, fusion proteins or monoclonal antibodies. Further examples are generally such disorders, in which an unwanted immune response is evoked or may be revoked by a therapeutic protein or peptide, e.g. in the course of a substitution therapy. Such therapeutic agents include rhEPO, rhMGDF/TPO, Glucocerebrosidase (Gaucher's), α-glucosidase (Pompe's), α-galactosidase A (Fabry's), IFN-α, IL-2, Factor II, Factor V, Factor VII, Factor VIII, Factor IX, Factor X, Factor XI, Factor XIII. Immune responses to therapeutic agents may also lead to undesired inflammations or even septic shock.

Of course, it is not required that the whole allergen, intolerance inducing agent, a target protein of an autoimmune response, e.g., of an autoantibody, or therapeutic agent is linked to the TCP. If T cell epitopes presented in an MHC of the subject to whom the TCP is to be administered are known, it is also possible to link one or more of said T-cell epitopes to the TCP.

Preferably, said allergen, intolerance inducing agent, target protein or target epitope of an autoimmune response or therapeutic agent and the TCP form a fusion protein.

The present invention also relates to a TCP fusion protein comprising a TCP dimer wherein the two TCP monomers are covalently linked with each other via one or two disulfide bridges in the hinge region, wherein the fusion protein may comprise, in each monomer, an agent against which an undesired immune reaction shall be suppressed and/or immunogenic tolerance is to be conferred, i.e., the dimer comprises two such agents.

In certain embodiments, the agent does not comprise a full variable domain of an immunoglobulin. Further, in certain embodiments, the fusion protein does not result in a full-length immunoglobulin.

However, in general, the agent linked is not limited in any way. Actually, one advantage of the frames identified is the possibility to easily reduce the immunogenicity of almost any antibody by inserting one or more heterologous tregitopes into the Fc-part of such antibody at the position corresponding to frames A, B, and C as mentioned above.

Thus, in certain embodiments, the TCP may be part of an antibody or a Fc-fusion protein. Preferably the antibody is a therapeutic antibody and the Fc-fusion protein is a therapeutic Fc-fusion protein. This will have the potential to reduce such antibodies' antigenicity and/or the formation of neutralizing anti-drug antibodies. Thus, an impairment of efficacy and/or an accelerated clearance of said antibody or Fc-fusion protein can be avoided. E.g., one may fuse the antigen binding regions or Fab fragments of the therapeutic antibody with the TCP. Alternatively, it is possible to integrate one or more heterologous tregitopes directly into the Fc-part of the therapeutic antibody according to the above described approach, preferably within at least one of frames A,B, or C. However, the TCP may also be fused with said antibody, preferably at the C-terminus of the heavy chain.

Suitable examples of therapeutic antibodies and therapeutic Fc fusion proteins are known to the skilled person. Examples for such therapeutic antibodies are Abciximab, Abrilumab, Adalimumab, Aducanumab, Afasevikumab, Afelimomab, Alemtuzumab, Anifrolumab, Anrukinzumab, Basiliximab, Belimumab, Benralizumab, Bertilimumab, Bevacizumab, Bleselumab, Blosozumab, Brazikumab, Brentuximab, Briakinumab, Brodalumab, Canakinumab, Catumaxomab, Cedelizumab, Certolizumab, Cetuximab, Clazakizumab, Clenoliximab, Crotedumab, Daclizumab, Denosumab, Dupilumab, Eculizumab, Eldelumab, Emicizumab, Enokizumab, Fasinumab, Fezakinumab, Fletikumab, Fulranumab, Gavilimomab, Gimsilumab, Golimumab, Guselkumab, Ibritumomab, Infliximab, Inolimomab, Ipilimumab, Itolizumab, Ixekizumab, Lebrikizumab, Letolizumab, Lulizumab pegol, Mavrilimumab, Mirikizumab, Muromonab-CD3, Natalizumab, Nemolizumab, Odulimomab, Ofatumumab, Olendalizumab, Olokizumab, Omalizumab, Opicinumab, Otelixizumab, Otilimab, Oxelumab, Ozoralizumab, Palivizumab, Panitumumab, Pascolizumab, Pembrolizumab, Perakizumab, Pertuzumab, Placulumab, Priliximab, Ranibizumab, Risankizumab, Rituximab, Rontalizumab, Sarilumab, Secukinumab, Sifalimumab, Siplizumab, Sirukumab, Talizumab, Tanezumab, Teplizumab, Tezepelumab, Tibulizumab, Tocilizumab, Tositumomab, Tralokinumab, Trastuzumab, Ublituximab, Ustekinumab, Vedolizumab, and Zanolimumab. Examples of therapeutic Fc fusion proteins include Abatacept, Alefacept, Belatacept, Etanercept, and Factor VIII-Fc-fusions and Factor IX-Fc fusions.

Therefore, the present invention also relates to an engineered antibody or Fc-fusion protein comprising a TCP according to the present invention, wherein preferably the TCP substitutes or essentially substitutes the Fc-part of said antibody or Fc-fusion protein. More particularly, the present invention also relates to an engineered antibody or Fc-Fusion protein comprising a tregitope carrying polypeptide (TCP) comprising an amino acid sequence having at least 85%, preferably, at least 90%, at least 95% or 100% sequence identity with amino acids 135 to 330 of SEQ ID NO: 1, wherein said TCP comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. The present invention also relates to an engineered antibody or Fc-Fusion protein comprising a tregitope carrying polypeptide (TCP) comprising an amino acid sequence having at least 85% preferably, at least 90%, at least 95% or 100% sequence identity with amino acids 114 to 330 of SEQ ID NO: 1, wherein said TCP comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. Said TCP may be a TCP as further defined above. The present invention also relates to an engineered antibody or Fc-Fusion protein comprising a tregitope carrying polypeptide (TCP) comprising an amino acid sequence having at least 85% preferably, at least 90%, at least 95% or 100% sequence identity with amino acids 104 to 330 of SEQ ID NO: 1, wherein said TCP comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. Said TCP may be a TCP as further defined above. The present invention also relates to an engineered antibody or Fc-Fusion protein comprising a tregitope carrying polypeptide (TCP) comprising an amino acid sequence having at least 85% preferably, at least 90%, at least 95% or 100% sequence identity with amino acids 1 to 330 of SEQ ID NO: 1, wherein said TCP comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. Said TCP may be a TCP as further defined above.

Preferably, any necessary effector functions of such therapeutic antibodies should be retained. For example, if a glyocsylation site is important, said glycosylation site should be maintained, and/or if binding to a Receptor is important, that should be maintained.
Multimers

The present invention also relates to a tregitope carrying polypeptide (TCP) multimer, preferably a TCP dimer, comprising at least two TCP monomers, each TCP monomer comprising an amino acid sequence having at least 85%, preferably, at least 90%, at least 95% or 100% sequence identity with amino acids 135 to 330 of SEQ ID NO: 1 or having at least 85%, preferably, at least 90%, at least 95% or 100% sequence identity with amino acids 1 to 330 of SEQ ID NO: 1, wherein each TCP monomer comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. Preferably, said TCP comprises sequences derived from a human Fc-part chain.

The invention also provides a TCP multimer, preferably a TCP dimer, comprising at least two TCP monomers, each TCP monomer comprising an amino acid sequence having at least 85%, preferably, at least 90%, at least 95% or 100% sequence identity with amino acids 114 to 330 of SEQ ID NO: 1 or having at least 85%, preferably, at least 90%, at least 95% or 100% sequence identity with amino acids 1 to 330 of SEQ ID NO: 1, wherein each TCP monomer comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity, e.g., as further defined above. The invention also provides a TCP multimer, preferably a TCP dimer, comprising at least two TCP monomers, each TCP monomer comprising an amino acid sequence having at least 85%, preferably, at least 90%, at least 95% or 100% sequence identity with amino acids 104 to 330 of SEQ ID NO: 1 or having at least 85%, preferably, at least 90%, at least 95% or 100% sequence identity with amino acids 1 to 330 of SEQ ID NO: 1, wherein each TCP monomer comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity, e.g., as further defined above.

The multimer may be a monomer, dimer, trimer, tetramer, pentamer, hexamer or of may comprise more than 6 TCP monomers. Preferably, a TCP multimer according to the present invention comprises from two to ten, more preferably from two to six monomers. In order to obtain a dimeric or multimeric form of the TCP, one may take advantage of the disulfide bonds formed via the hinge region of the immunoglobulin Fc-part chain.

The skilled person understands that the TCP monomers in such TCP multimers do not need to be completely identical. However, in a TCP multimer according to the present invention the monomers should have a substantially similar structure. Preferably each monomer in a multimer should have at least 60%, preferably 65%, 70%, 75%, 80%, 85%, more preferably 90%, 95% sequence identity to each other monomer comprised in the multimer. If the monomers are (substantially) identical (such as having more than 95%, more particularly more than 97% amino acid sequence identity), they may be identified with the prefix “homo” (such as in homodimer). If the different monomers in the multimer show relevant differences (e.g. the TCP monomers comprise at least one different tregitope), they may be identified with the prefix “hetero” (such as in heterodimer).

Particularly preferred are TCP dimers. Accordingly, the present invention also relates to a TCP as described herein, wherein said TCP forms a dimer comprising at least two TCP monomers as described herein. The present invention thus also provides a TCP dimer comprising two TCP monomers. For example, the TCP monomers may de covalently bound via at least one disulfide bridge, preferably two disulfide bridges. Preferably, said TCP monomers each comprise a hinge region derived from an immunoglobulin or a part thereof enabling dimer formation. More specifically, said TCP monomers each comprise at least a part that enables dimer formation, optionally, having at least 50%, at least 60%, at least 70%, at least 90% or 100% sequence identity to amino acid positions 103 to 113 of SEQ ID NO: 1, wherein preferably, the cysteine residues in amino acid positions 109 and 112 of SEQ ID NO: 1 are retained. A partial hinge region enabling dimerization has at least 85%, preferably, 100% sequence identity to amino acids 104-113 of SEQ ID NO: 1.

The TCP may form a dimer (comprising two TCP monomers) dimerized via one or more (e.g., two) disulfide bridges, preferably, the TCP forms a dimer of two TCP monomers dimerized via the hinge region of an immunoglobulin. As known by the skilled person, an Fc-fragment obtainable by papain digestion of an immunoglobulin typically is a dimer. Similarly, if the hinge region of the Fc-part chain is included in the TCP, the TCP is likely to spontaneously dimerize via the respective hinge regions. Dimerization may further be supported by non-covalent CH3-CH3-interactions.

Alternatively, in said TCP multimer, the TCP monomers may be covalently or non-covalently bound to each other, e.g. as fusion proteins or via a flexible or non-flexible linker. Dimerisation may also be effected, e.g., via a leucine zipper.

Such TCP dimers generally tend to have an improved stability and half-life compared to the corresponding monomers. The TCP dimer may be a homodimer or a heterodimer. A homodimer may be easier to manufacture in a reliable manner using cellular or protein-free expression systems. On the other hand, a heterodimer may have the advantage of being capable of integrating more different tregitopes, e.g. up to eight different tregitopes (in each monomer one tregitope in each of frames A,B,C, and one C-terminal tregitope).

Techniques to generate heterodimers are generally known to the skilled person. In addition to co-expression of different TCP monomers, formation of said heterodimers can be induced by certain modifications of the TCPs. Such modifications are e.g. known from heavy chain-heavy chain pairings of bispecific antibody formats such as (I) disulfide bond pairing by introduction of cysteine pairs into the region of the TCP corresponding to the CH3 domain of the immunoglobulin Fc-part chain, (II) introducing charged residues facilitating salt bridges by oppositely charged residues for the different TCP monomers, and (III) the knobs-into-holes (KiH) strategy based on the substitution of either smaller or respectively larger amino acids in the different TCP monomers. In particular, the KiH strategy is very efficient.

TCP hexamers can also be formed. Engineered hexavalent Fc proteins are known in the art (Rowley et al. 2018. Communications Biology 1:146).

As described herein, a TCP multimer may comprise either TCP proteins essentially not comprising sequences other than the tregitopes and the sequences with at least 85% sequence identity to certain regions of SEQ ID NO: 1, or they may comprise TCP proteins that further comprise other immunoglobulin domains, e.g., antigen-binding parts of antibodies, or other polypeptides, e.g., polypeptides to which an immune response is to be modulated. Of course, mixed multimers can also be generated.
Monomers

Nevertheless, there are applications wherein dimer or multimer formation is not preferred. Preventing dimer or multimer formation may be desirable, e.g. if the TCP sequence is combined and/or fused to another agent as outlined in more detail elsewhere. For example, the cysteine residues of the hinge region may undesirably interact with a fusion partner in a fusion protein comprising the TCP.

In such cases, it may be advisable to avoid dimer formation or other undesired disulfide bridges via the hinge region. For example, in such case, the TCP should not comprise the hinge region. Alternatively, the cysteine residues responsible for dimerization may be deleted or substituted as known in the art. For example, the one or more of the respective cysteine residues might be substituted with serines or other amino acids, thereby preventing dimer formation of the resulting molecule. Alternatively, formation of dimers or multimers may be eliminated after expression and purification by chemical reactions e.g. reduction and subsequent alkylation. For example, cysteine based disulfide bonds of the hinge region can be broken by reductive reactions, e.g. using reducing agents such as reduced glutathione, 2-mercaptoethylamine, dithiothreitol or tris-2-carboxyethylphosphine hydrochloride. Subsequent alkylation using reagents such as iodoacetamide can prevent reformation of disulfide bonds between cysteines.

Nucleic Acids, Host Cells and Transgenic Animals

The invention also provides a nucleic acid encoding the TCP according to the invention, as specified herein, e.g., a nucleic acid encoding a tregitope carrying polypeptide (TCP) comprising an amino acid sequence having at least 85% sequence identity with amino acids 135 to 330 of SEQ ID NO: 1, wherein said TCP comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity. The TCP may comprise sequences derived from a human Fc part-chain. The invention also provides a nucleic acid encoding the TCP according to the invention, as specified herein, e.g., a nucleic acid encoding a tregitope carrying polypeptide (TCP) comprising an amino acid sequence having at least 85% sequence identity with amino acids 114 to 330 of SEQ ID NO: 1, wherein said TCP comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity, e.g., as defined above. The invention also provides a nucleic acid encoding the TCP according to the invention, as specified herein, e.g., a nucleic acid encoding a tregitope carrying polypeptide (TCP) comprising an amino acid sequence having at least 85% sequence identity with amino acids 104 to 330 of SEQ ID NO: 1, wherein said TCP comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity, e.g., as defined above. The invention also provides a nucleic acid encoding the TCP according to the invention, as specified herein, e.g., a nucleic acid encoding a tregitope carrying polypeptide (TCP) comprising an amino acid sequence having at least 85% sequence identity with amino acids 1 to 330 of SEQ ID NO: 1, wherein said TCP comprises at least one tregitope heterologous to SEQ ID NO: 1, said heterologous tregitope being located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity, e.g., as defined above.

The TCP encoded by the nucleic acid may, e.g., be a fusion protein. Preferably, the nucleic acid encodes a TCP including a signal peptide to allow for secretion of the expressed protein, and accordingly, easier purification. In a preferred embodiment, the sequence encoding the TCP is functionally linked to a sequence encoding an N-terminal signal peptide having such as an eukaryotic signal peptide, e.g., an amino acid sequence as shown in SEQ ID NO: 22

(METDTLLLWVLLLWVPGSTG).

The nucleic acid may be a vector suitable for homologous recombination in a prokaryotic or eukaryotic host cell, preferably an eukaryotic host cell. For example, the vector may be suitable for CRIPR/Cas based recombination.

The nucleic acid may also be an expression vector. Thus, the present invention also relates to an expression vector comprising the nucleic acid encoding the TCP. More particularly, the expression vector shall be suitable for expressing the TCP in a eukaryotic or prokaryotic host cell. Again, it is to be understood that said expression vector may comprise a nucleic acid encoding the TCP in any of the embodiments disclosed throughout this specification, including as a TCP fusion protein.

Suitable expression vectors to generate such expression constructs are well-known to the skilled person. Depending on the respective expression system and the respective cell it may be preferred to codon-optimize the expression construct and/or to clone it into a suitable vector.

Preferably, in the expression vector, the nucleic acid is functionally linked to a suitable promoter. Such promoters are generally known in the art. The promotor may be constitutive or inducible. Preferably, the promotor is suitable for mediating expression of the TCP in a host cell, in particular, in a eukaryotic host cell. It may also be suitable for expression in a transgenic animal, e.g., in a human. For example, the promotor may be a tissue-specific promotor. For example, a promotor suitable for expression in a bird egg may be chosen. The promotor may also be able to mediate expression in cells that leads to secretion into the milk in a milk-producing animal, such as a cow, sheep, goat or camel. The promotor may also be a tissue-specific promotor capable of mediating expression in human cells expressing an antigen to which there is an autoimmune-reaction and/or in antigen-presenting cells such as dendritic cells, macrophages and/or B-cells.

The present invention also provides a eukaryotic or prokaryotic host cell, comprising the nucleic acid, e.g., the expression vector, encoding a TCP according to the present invention, wherein the host cell preferably is capable of expressing said TCP.

Preferably, the cell is a eukaryotic cell. The cell may be a mammalian cell. For example, for therapeutic applications, it is advantageous to use a eukaryotic and preferably a mammalian cell, as the TCP produced may be more similar to human proteins, e.g., in view of post-translational modifications like glycosylation. Suitable host cells are known. For example, the cell may be an epithelial cell, a monocyte-derived cell, e.g., a macrophage, a dendritic cell, a B cell, an islet cell or a fibroblast cell. More specific examples are HEK 293, CAP-T cell, CAP-Go, CHO (e.g. CHO DG44), COS (e.g. COS-1 or COS-7), BHK-21, Jurkat, Peer, CML T1, EL4, T2, HeLa, MDCKII, and Vero. In certain preferred embodiments, the cell is a HEK293 cell or a CAP-T cell or a CAP Go cell.

The invention further provides a transgenic, preferably, non-human animal comprising the nucleic acid of the present invention, e.g., a mouse, a rat, a rabbit, a guinea pig, a monkey, an ape, a pig, a do, a cat, a camel, a cow, a sheep, a goat or a bird such as a chicken. The transgenic animal preferably is capable of expressing said TCP in one or more cells or tissues. For example, a female transgenic animal, e.g., a camel, cow, sheep or goat may be capable of secreting the TCP in its milk. A transgenic bird may also be capable of laying eggs comprising the TCP of the invention. Such transgenic animals may thus be used for producing the TCP of the invention. They may also be used, e.g., for research.

Methods of Manufacturing

The present invention provides a method of manufacturing a nucleic acid encoding a TCP of the present invention. Said method may comprise steps of
(a) providing a nucleic acid sequence encoding an immunoglobulin (e.g., IgG1, IgG2, IgG3, IgG4, IgG5, IgA, IgD, IgE, preferably, IgG1) Fc-part chain or a part thereof, e.g., a wt Fc part chain,
(b) introducing nucleic acid sequences of one or more, e.g., two, three or four heterologous tregitopes into the nucleic acid sequence of step (a) preferably, at a position corresponding to one or more of frames A, B, or C of the immunoglobulin Fc-part chain according to SEQ ID NO: 1 as defined herein,
(c) generating a nucleic acid having the sequence of step (b).

The present invention also provides the use of the nucleic acid sequence of an immunoglobulin Fc-part chain for manufacturing a nucleic acid encoding a TCP or a nucleic acid encoding a protein comprising said TCP.

The nucleic acid sequences encoding said TCP can be designed manually or in silico, optionally, followed by recombinant or chemical synthesis of the nucleic acid encoding said TCP or protein. Suitable methods are known and available to the skilled person.

The present invention provides a method of manufacturing a TCP of the present invention. The TCP according to the invention is an artificial or engineered protein, it does not occur in nature. The TCP may be produced by any method of protein synthesis deemed appropriate by the skilled person, be it recombinant or non-recombinant techniques. For example, the TCP may be produced by expression in cell culture or by chemical protein synthesis. However, recombinant expression in a cellular or cell-free system is preferred due to the well-established methodology and comparatively low costs. In recombinant expression, the good expression level achievable with the TCP according to the invention is a particular advantage.

Suitable methods for cloning, expression and purification of the TCP can be taken e.g. from laboratory manuals like J. Sambrook and D. Russel, Molecular Cloning: A Laboratory Manual, 3. Edition, Cold Spring Harbour Laboratory Press, Cold Spring Harbor, N.Y. (2001).

The invention also provides a method for manufacturing a TCP as described herein, e.g. comprising steps of (a) generating a suitable expression vector comprising a nucleic acid encoding the TCP, (b) transfecting a suitable host cell with said expression vector, (c) culturing said host cell under conditions allowing for expression of said TCP, (d) isolating said TCP.

In particular, the invention provides a method of manufacturing a TCP, comprising steps of
(a) cultivating the host cell of the invention under conditions suitable for expression of the TCP;
(b) harvesting the cell or medium comprising the TCP expressed in step (a);
(c) isolating said TCP;
(d) optionally, formulating the TCP of step (c) in a pharmaceutically acceptable composition.

Moreover, optionally the protein of step c) or the composition of step d) may be filled into a suitable container, e.g., a syringe.

The TCP of the invention may be isolated from cells, in particular, if the TCP is expressed without a signal sequence. It may also be isolated from medium, in particular, if the TCP is expressed with a signal sequence for extracellular secretion.

Isolation, in the context of the invention, may mean purification to varying degrees of purity. Isolation eliminates or reduces at least one non-TCP component from the cell or medium. For example, purity of the TCP may be at least 50%, at least 60%, at least 70%, at least 80%, at least 90%, at least 95%, at least 99% or at least 99.5%, wherein percentages relate to w/w. Applicable isolation or purification methods and steps are known to the skilled person and may be applied as deemed appropriate. Examples include ion exchange chromatography, hydrophobic interaction chromatography, affinity chromatography, filtration, nanofiltration, precipitation (e.g. ethanol precipitation), ultrafiltration, and/or diafiltration. Depending on the needed purity, different isolation steps may be combined, such as primary purification, intermediate purification, and polishing. In addition, the purified TCP may subsequently concentrated and formulated into a suitable buffer or a pharmaceutical composition, e.g. using ultrafiltration and/or diafiltration. The method may also comprise sterilization, e.g., by irradiation or sterile filtration, in particular, for therapeutic applications.

For example, if the TCP binds to protein A or protein G, the affinity chromatography may be based on protein A or protein G.

An isolation method based on affinity comprising the use of a polyclonal or monoclonal antibody directed against the immunoglobulin Fc-part chain sequences included in the TCP of the invention was found to be particularly useful for isolation. Thus, for example, step (c) may comprise adsorbing the TCP on an affinity material, wherein said affinity material preferably includes a polyclonal antibody to the Fc-part of human Ig, wherein step (c) optionally includes an affinity chromatography. A monoclonal antibody is usually better controllable than a polyclonal antibody. In contrast, a polyclonal antibody has the advantage of recognizing different TCPs irrespective of their particular sequence. Thus, either a polyclonal or monoclonal antibody may be useful for isolating the TCP of the invention.

Alternatively, if the TCP comprises an affinity tag, it may be isolated based on said affinity, e.g., via a metal chelation affinity matrix, e.g., a Ni²⁺ affinity matrix, for a His-tag. An antibody directed against an affinity tag on the TCP, e.g., a FLAG tag, may also be employed for isolation. An affinity-based isolation may include an affinity chromatography. Affinity adsorption may be carried out in column or batch form,

It is clear for the skilled person that for all these uses and methods mentioned above, the preferred TCPs and features of the TCP described elsewhere apply analogously, including but not limited to the possible or preferred Tregitopes, Fc-part chains, sequence frames, and the rules for integration and location. Analogously, multimers and fusion proteins comprising the TCP can be designed and manufactured.

Uses of the TCP

The TCP according to the present invention is useful in multiple ways, including:
1) for expression and production of isolated tregitopes, e.g. by enzymatic excision and subsequent purification of the tregitopes from the TCP produced,
2) for use as a stand-alone therapeutic, e.g. as a TCP monomer or multimer, particularly a TCP dimer,
3) for use in co-administration with agents against which an immune response shall be suppressed and/or tolerance induced, wherein the co-administration may be in a form wherein the TCP is not linked to the agent or in a form wherein the TCP is non-covalently or covalently linked to the agent, e.g., for use in fusion proteins comprising the TCP, particularly for suppressing an immune response against the non-TCP fusion partner in such fusion protein and/or induction of immune tolerance to said fusion partner, or
4) for in vitro methods, e.g., for in vitro activating regulatory T cells, or
5) for use in research.

In the first application, the TCP can be used for producing tregitopes. Advantageously, the TCP according to the present invention facilitates production of tregitopes, in particular recombinant production. Thus, the present invention provides a method for manufacture of a polypeptide or peptide comprising or consisting of one or more tregitopes, comprising the steps of
a) providing a TCP according to the invention,
b) excising a peptide or polypeptide comprising one or more tregitopes from the TCP,
c) optionally purifying the peptide or polypeptide from step b)

Step a) may comprise the steps of manufacturing a TCP of the invention as described herein. Step b) may be carried out by any means or method, e.g. by chemical or enzymatic excision. Advantageously, the tregitope(s) comprised in the TCP may be flanked by enzymatic cleavage sites allowing for defined excision of the polypeptide(s) or peptide(s) comprising or consisting of the tregitope(s). Preferably, “flanked” in this context means that the enzymatic cleavage site is located in close proximity to the tregitope, more preferably less than 20, less than fifteen, or less than 10 amino acid residues away from the most proximal end of the tregitope.

It will be understood by the skilled person that it may not be necessary to provide a peptide consisting just of the tregitope. It may be entirely sufficient or even advantageous to provide a peptide or polypeptide comprising further amino acids. Thus, e.g. enzymatic recognition and/or cleavage sites may be chosen and included more flexibly, e.g. in the region flanking the tregitope(s). In addition, the peptide or polypeptide may comprise e.g. additional useful amino acid residues, e.g. a purification tag as mentioned elsewhere. Preferably the enzymatic recognition and/or cleavage site is located within one of said sequence frames A, B, or C of the TCP as described in more detail elsewhere in this specification. Preferably, any tag linked to the tregitope is also located within one of said sequence frames A, B, or C.

Consequently, the present invention also provides a peptide or polypeptide comprising or consisting of one or more tregitopes obtainable according to the present invention.

The peptide or polypeptide comprising or consisting of one or more tregitopes may be purified by any method deemed appropriate. Suitable methods are known and include, e.g., ion exchange chromatography, hydrophobic interaction chromatography, affinity chromatography, filtration, nanofiltration, precipitation (e.g. ethanol precipitation), ultrafiltration, diafiltration. Suitable methods for purification of tregitopes have also been described in WO 2008/094538A2.

As shown in the examples below, the expression level of the TCP of the invention is much higher than expression of single tregitopes. Thus, the presently disclosed route of preparation of tregitopes from TCP is advantageous.

Medical uses as a stand-alone therapeutic or for use in co-administration with agents against which an immune response shall be suppressed and/or tolerance induced are described in more detail below.

The TCP of the invention may also be used in vitro. For example, the invention provides a method for modulating an immune response, preferably, for suppressing an immune response or inducing tolerance, e.g., in vitro, comprising contacting immune cells such as antigen presenting cells (e.g., dendritic cells, macrophages and/or B-cells) and/or T-cells, with a TCP according to the invention, a nucleic acid according to the invention, or a host cell according to the invention, wherein, optionally, said immune response is an immune response to an agent with which the TCP is covalently or non-covalently linked, or with which the TCP, nucleic acid or host cell is mixed or contacted at substantially the same time.

For example, the invention provides a method for activating regulatory T cells isolated from a patient. Regulatory T cells activated in this manner may be for use in administration to said patient, e.g., for use in suppressing an undesired immune reaction and/or conferring immunogenic tolerance. For example, such regulatory T cells may recognize the tregitope(s) provided in the TCP and/or epitopes comprised in an agent linked to said TCP, e.g., a protein against which an undesired immune reaction is to be suppressed and/or immunogenic tolerance is to be conferred. The invention thus also provides a method for suppressing an undesired immune reaction and/or conferring immunogenic tolerance to an agent, comprising isolating T cells from a subject (to any degree of purity, e.g., the T cells may also be in a composition containing antigen presenting cells such as dendritic cells, macrophages and/or B cells from the subjects, e.g., in the context of PBMC), contacting said T cells with TCP of the invention under conditions suitable for activating said T cells, optionally, isolating regulatory T cells activated, and administering said T cells, preferably, said regulatory T cells to said subject. Conditions suitable for activating said T cells typically require the presence of antigen presenting cells, preferably, professional antigen presenting cells such as dendritic cells, macrophages and/or B cells. Said antigen-presenting cells may also be host cells of the invention. Typically, the cells are co-incubated for a suitable time, e.g., 12-36 h, optionally, 16-24 h. Before T cells are re-administered to the subject, the characteristic, e.g., the cytokine production and/or immune-specific marker proteins (e.g. CD25, CD127,FoxP3, CD45RA, CCR7) of the T cells may be analysed, for example using FACS or ELISA. Preferably, T cells having a regulatory phenotype, e.g., expressing CD25, CD127,FoxP3, CD45RA, CCR7 and/or IL-10, are administered.

The TCP of the invention may also be used for research, e.g., in animal models, and/or for toxicity tests, and/or for stimulation of isolated primary cells for cell culture based experiments.

Compositions and Kits

As noted herein, the TCP of the invention may be co-administered with an agent, wherein the agent optionally is an agent against which an undesired immune reaction is to be suppressed and/or immunogenic tolerance is to be conferred. Thus, the invention provides a composition comprising a TCP of the invention, which may be a monomer or a multimer (e.g., a dimer), wherein said composition further comprises an agent, wherein the agent optionally is an agent against which an undesired immune reaction is to be suppressed and/or immunogenic tolerance is to be conferred.

Said agent may be, e.g., an allergen, an intolerance inducing agent, a target protein of an autoimmune response, in particular, of an autoantibody, or a target epitope of an autoimmune response, in particular, of an autoantibody, or a target epitope of a T-cell mediated autimmune response, or a therapeutic agent.

The invention provides compositions suitable for different kinds of co-administration.

Firstly, the invention provides a composition comprising a TCP of the invention, wherein the TCP is not linked to the agent. Accordingly, the TCP and the agent are merely mixed, not associated with each other. For example, the composition may be a solution, preferably, a homogenous solution. The TCP may be a multimeric TCP, e.g., a dimer. Alternatively, it may be in monomeric form.

Secondly, the invention provides a composition comprising a TCP of the invention, wherein the TCP is non-covalently linked to the agent. Such a non-covalent association may be an unspecific interaction, e.g., via hydrophobic interactions, van-der-Waals-interactions or polar interactions. It may also be a specific interaction, e.g., if the TCP is an antibody comprising an antigen-binding part of an antibody, said antigen-binding part may specifically bind to said antigen, wherein the antigen is the agent. This may be particularly useful if the agent is an allergen or a protein which is the target of an autoimmune response, in particular, of an autoantibody. The TCP may be a multimeric TCP, e.g., a dimer. Alternatively, it may be in monomeric form.

Thirdly, the invention provides a composition comprising a TCP of the invention, wherein the TCP is covalently linked to the agent, e.g., in the form of a fusion protein. The TCP may be a multimeric TCP, e.g., a dimer. Alternatively, it may be in monomeric form.

In a fourth embodiment, the composition does not comprise another active agent, such as an agent against which an undesired immune reaction is to be suppressed and/or immunogenic tolerance is to be conferred. Again, said TCP may be a multimeric TCP, e.g., a dimer. Alternatively, it may be in monomeric form.

In any of these forms, the composition may further comprise pharmaceutically acceptable excipients, as further described below.

The invention also provides a kit comprising, separately, a TCP of the invention, and an agent, optionally, an agent against which an undesired immune reaction is to be suppressed and/or immunogenic tolerance is to be conferred. Said agent may be, e.g., an allergen, an intolerance inducing agent, a target protein of an autoimmune response, in particular, of an autoantibody, or a target epitope of an autoimmune response, in particular, of an autoantibody, or a therapeutic agent. Such a kit may further comprise suitable excipients for formulating a pharmaceutical composition. It may contain instructions for the medical use. The kit may also comprise an outer package comprising one or more containers comprising the TCP or pharmaceutical composition and the instructions, optionally further comprising one or more devices for e.g. for reconstitution and/or administration of the protein(s) of the invention.

A kit of the invention may alternatively or additionally comprise means for linking the TCP and the agent, e.g., via chemical linkage. In that form, it also typically comprises instructions for linking the TCP and the agent. A kit of the invention may also comprise the TCP and means for linking the TCP and an agent not provided in the kit, e.g., by chemical linkage, and optionally, instructions for linking the TCP and the agent. Suitable linkers are known in the art.

Pharmaceutical Compositions

The invention provides a pharmaceutical composition comprising the TCP of the present invention, a nucleic acid of the present invention (in particular, a host cell suitable for expression in a human cell), or a host cell of the present invention. Preferably, the pharmaceutical composition comprises a TCP of the present invention.

The pharmaceutical composition optionally comprises at least one pharmaceutically acceptable excipient. Optionally, between one and ten pharmaceutically acceptable excipients, more preferably between one and five pharmaceutically acceptable excipients. The term “excipient” also comprises carriers and/or diluents. Suitable excipients are generally known to the skilled person. Examples are salts, buffers, preservatives and osmotically active substances. Examples of the carrier include but are not limited to phosphate buffered saline, Ringer's solution, dextrose solution, water, emulsions, such as oil/water emulsions, various types of wetting agents, sterile solutions, etc. The composition may further comprise an appropriate amount of a pharmaceutically acceptable salt to render the composition isotonic. Preferably, acceptable excipients, carriers, or stabilisers are non-toxic at the dosages and concentrations employed. They may include buffers such as citrate, phosphate, and other organic acids; salt-forming counter-ions, e.g. sodium and potassium; low molecular weight polypeptides (e.g. more than 10 amino acid residues); proteins, e.g. serum albumin, or gelatine; hydrophilic polymers, e.g. polyvinylpyrrolidone; amino acids such as histidine, glutamine, lysine, asparagine, arginine, or glycine; carbohydrates such as glucose, mannose, or dextrins; monosaccharides; disaccharides; other sugars such as sucrose, mannitol, trehalose or sorbitol; chelating agents such as EDTA; non-ionic surfactants such as Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol. Moreover, the pharmaceutical composition may comprise one or more stabilizers. Typical examples are amino acids (such as glycine, glutamate, or histidine), sugar or sugar alcohols (such as trehalose, sorbitol, mannitol), detergents (such as polysorbate, or poloxamer). Suitable excipients and formulations are described in more detail in Remington's Pharmaceutical Sciences, 17th ed., 1985, Mack Publishing Co. The choice of excipient and/or carrier and/or diluent may depend upon route of administration and concentration of the active agent(s), preferably, the TCP of the invention and, optionally, as described herein, a further agent which may be co-administered. The pharmaceutical composition may be in any form deemed suitable, in particular it may be liquid or lyophilized. The pharmaceutical composition may be formed e.g. into tablets, pills, capsules, suppositories, suspensions, lozenges, powders, liquids, aqueous solutions or lyophilised compositions for solubilisation and the like, preferably, an aqueous solution or a lyophilised composition.

The pharmaceutical composition may be formulated for parenteral, e.g., intravenous, subcutaneous, oral, topical, rectal, nasal administration or any other administration route. Intravenous or subcutaneous administration is preferred. In a clinical setting, intravenous administration may be preferred. Subcutaneous administration can more easily be performed at home. The skilled person can chose the administration mode depending on the facts of the case. The preferred mode of administration will also depend, e.g., on the subcutaneous availability of the TCP.

For example, in particular if the pharmaceutical composition comprises the TCP as stand-alone agent, any excipients, diluents and carriers typically used for immunoglobulin preparations, such as pharmaceutical compositions comprising monoclonal or polyclonal antibodies, in particular intravenous or subcutaneous immunoglobulin preparations, are also be suitable for pharmaceutical preparations comprising the TCP. For example, the pharmaceutical preparation may comprise the TCP at a concentration of between 1 and 50 g/I, an amino acid (such as 150 to 500 mM glycine), optionally a detergent (such as 20 mM polysorbate), and a buffer, at pH from 4.3 to 6.5.

The dosage of the TCP formulated for administration may be chosen depending on the specific disorder to be treated and the administration route. The skilled person is aware of means and methods for finding suitable safe and effective dosages.

As a guidance, the dosage may be e.g. in the range of 2 mg/kg bodyweight up to 20 g/kg bodyweight, especially in the range of 200 mg/kg bodyweight up to 10 g/kg bodyweight.

As a further guidance: If the TCP is administered as a stand-alone therapeutic, e.g. in context with an autoimmune disorder, the dosage may be in a range corresponding to dosages of polyclonal intravenous or subcutaneous immunoglobulins (IVIG) used to treat the respective disorder. If the TCP is co-administered with an agent against which an immune response shall be suppressed, the dose will rather be in molar excess to the dose of said agent. If the TCP is is linked with a therapeutic agent, e.g., forming a fusion protein with a therapeutic protein, the dosage will be mostly determined by the effective dose appropriate for said therapeutic agent.

Depending on the disorder to be treated or prevented, the TCP may be applied as a single dose or in multiple dosages. For example, administration may be daily, bi-daily, weekly, bi-weekly, or monthly. Administration may also be continuously, e.g. via a suitable pump. Advantageously, the TCP typically has a good plasma half-life due to its Fc-part-derived backbone sequences. Thus, it is preferably sufficient to administer the TCP weekly, bi-weekly, or even monthly. Multimers such as dimers may have a particularly long plasma half-life allowing for weekly, bi-weekly, or even monthly administration. The precise plasma half-life of a particular TCP can be determined by the skilled person by methods known in the art, such as by suitable pharmakokinetic studies. The plasma half-life will also depend on whether binding of the TCP to the neonatal Fc-receptor (FcRn) is retained or not. This can be tested by the skilled person. Similarly, the plasma half-life can be extended or shortened by the skilled person. For example, as mentioned, the TCP may be PEGylated in order to extend the plasma half-life.

The pharmaceutical composition comprising the TCP is applicable for any of the therapeutic uses described herein.

Preferably, the pharmaceutical composition is a stable composition, i.e. the TCP remains suitable for administration to a patient for at least 3 months, more preferably at least 6 months, if stored at 2 to 8° C., more preferably if stored at room temperature (18° C. to 25° C.).

The TCP or the pharmaceutical composition may be comprised in a suitable container, e.g., a flask, a bottle, a bag, or a syringe. For use in medicine, the pharmaceutical composition comprising the TCP preferably is in a pharmaceutically acceptable container.

The pharmaceutical composition of the invention may be a composition comprising a TCP of the invention in the absence or presence of a further agent, such as an agent against which an undesired immune reaction shall be suppressed and/or tolerance conferred, in all forms described herein. The invention also relates to a pharmaceutical composition comprising a combined composition comprising a TCP according the invention and an agent capable of eliciting an undesired immune response.

The invention also relates to a pharmaceutical kit comprising a TCP according the invention (which may be a multimer, e.g., a dimer, or a monomer) and, separately, an agent against which an undesired immune reaction shall be suppressed. The kit may also comprise instructions for medical use of said kit, e.g., with instructions for dosages and administration routes described elsewhere in this specification.

Said kit may be for use in co-administration of these components, wherein co-administration may be at the same or a similar site or to a similar compartment of the subject to be treated with the kit. For example, co-administration may be intravenous administration of both components, e.g., into different veins. Preferably, co-administration is at the same site.

Co-administration may also be at the same or substantially the same time, e.g., within one day, preferably, within one hour or less, e.g., within 10 minutes, within 5 minutes, or within 1 minute. Co-administration advantageously has the effect that the components of the kit, i.e., the TCP and the agent are presented to T cells at substantially the same time, so that T cells reacting to epitopes of the agent are influenced by regulatory T cells activated by the tregitopes derived from the TCP, thus suppressing an immune response to the agent and/or conferring immunity to the agent.

Medical Indications

The TCP according to the present invention is useful in medicine. Due to the presence of tregitopes, the TCP has immunomodulatory and immunosuppressive properties which can be beneficially used in medicine in multiple ways.

In particular, the invention provides the pharmaceutical composition of the invention for use in modulating an immune response in a subject. The invention also discloses a method for modulating an immune response in a subject in need thereof, comprising administering a pharmaceutical composition of the invention to the subject. Modulation of an immune response can be suppressing an immune response or inducing tolerance (i.e., conferring tolerance).

The invention also provides the pharmaceutical composition of the invention for use in suppressing an immune response or inducing tolerance. The invention also discloses a method for suppressing an immune response or inducing tolerance in a subject in need thereof, comprising administering a pharmaceutical composition of the invention to the subject.

The invention also provides the pharmaceutical composition of the invention for use in the prevention or treatment, preferably, treatment, of an autoimmune related disorder, allergy, viral infection, or transplantation-related immune reaction or disorder. Likewise, the present invention further relates to the use of a TCP according to the present invention for the preparation of a medicament for the prevention or treatment of autoimmune related disorders, allergy, viral infection, or transplantation-related immune reactions or disorders. The present invention also provides a method of preventing or treating an autoimmune related disorder, allergy, viral infection, or a transplantation-related immune reaction or disorder in a subject in need thereof, comprising administering a pharmaceutical composition of the invention to the subject.

“Treatment” in the context of the invention means that at least one symptom of the disease is ameliorated, wherein, preferably, more than one, most preferably, all symptoms of the disease are ameliorated or the symptoms do not occur any more. Treatment can be repeated, if desired. “Prevention” includes reduction of the risk or incidence of occurrence of a disease.

The term “subject” as used herein relates to a human or non-human mammal, preferably a human subject. The subject may be a patient, e.g., suffering from one or more of the diseases or disorders as mentioned herein. The term non-human mammal is not limited in a particular way, and includes, e.g., a dog, a cat, a horse, a sheep, a goat, a cow, a camel, a guinea pig, a pig, a rabbit, a mouse or a rat. For therapeutic use, the Fc-part chain and/or the tregitopes used for the TCP are preferably derived from the species to be treated.

“Autoimmune related disorders” encompass neurological autoimmune disorders, dermatological autoimmune disorders, rheumatoid disorders, metabolic disorders, thyroid diseases, transplant-related immune reactions and disorders, and other autoimmune disorders.

Examples of neurological autoimmune disorders are demyelinating diseases, such as chronic inflammatory demyelinating polyneuropathy (CIDP), multifocal motor neuropathy (MMN), Guillain-Barré-syndrome, multiple sclerosis (MS), neuromyelitis optica, acute disseminated encephalomyelitis, myasthenia gravis, Lambert-Eaton syndrome, anti-NMDAR encephalitis, stiff-person syndrome, neurodegenerative central nervous system diseases, IgM-associated polyneuropathy, myositis, autoimmune polymyositis, inclusion body myositis, immune neuromyotonia, chronic focal encephalitis, and pediatric autoimmune neuropsychiatric disorders associated with Streptococcal infection (PANDAS).

Examples of dermatologic autoimmune disorders include blistering dermatologic disorders, such as pemphigus (e.g. pemphigus vulgaris and pemphigus foliaceus), autoimmune dermatomyositis, pyoderma gangraenosum, toxic epidermal necrolysis (TEN), Stevens-Johnson-syndrome (SJS), atopic dermatitis, autoimmune urticaria, and scleromyxoedema.

Examples for rheumatoid disorders are rheumatoid arthritis (RA), juvenile rheumatoid arthritis, psoriasis, and systemic lupus erythematodes (SLE).

Metabolic autoimmune disorders include, for example, type I diabetes.

Examples for autoimmune related disorders of the thyroid disease type are Graves' disease, and autoimmune thyroiditis.

Other autoimmune disorders are, e.g., primary and secondary vasculitis, such as Kawasaki syndrome, microscopic polyangiitis, Wegener's granulomatosis, Churg-Strauss-syndrome, IgA-associated vasculitis, polyarteritis nodosa, livedoid vasculopathy, antiphospholipid antibody syndrome (APS), paraneoplastic syndromes, and immune thrombocytopenia (ITP). Further conceivable is the treatment or prevention of autoimmunehemophilia (such as autoimmune hemolytic anemia), autoimmune hepatitis, autoimmune asthma, and neurodermitis, thrombotic thrombocytopenic purpura (TTP), and chronic pain.

In the context of treatment of autoimmune disorder, it may be advantageous if the TCP is co-administered (e.g., covalently linked, such as in the form of a fusion protein) with an agent, typically, a protein known to be the target or the autoimmune response. However, as explained herein, that is not required.

Likewise, allergies or intolerances like food intolerances may be treated according to this aspect of the invention. For example, in this embodiment, it may be advantageous that the TCP is co-administered (e.g., covalently linked, such as in the form of a fusion protein) with the allergen or parts of the allergen or substances or compounds responsible for the intolerance, if these are known. However, as explained herein, that is not required.

Examples of viral infections that may be targeted by the pharmaceutical compositions of the present invention are Hepatitis B infection and Hepatitis C infection. For example, acute exacerbations of chronic Hepatitis B may be accompanied by increased cytotoxic T cell responses to Hepatitis B core and e antigens (HBcAg/HBeAg), and regulatory T cells specific for HBcAg decline during exacerbations, accompanied by an increase in HBcAg peptide-specific cytotoxic T cells, see also WO 2008/094538 A2. Thus, an increased activation of regulatory T cells mediated by the treatment of the present invention may help to reduce such exacerbations or symptoms thereof. In this embodiment, it may be advantageous that the TCP is co-administered (e.g., covalently linked, such as in the form of a fusion protein) with the antigen to which increased responses are seen or parts thereof, if these are known. However, as explained herein, that is not required.

“Transplant-related immune reactions and disorders” are, for example, transplant rejection, host versus graft disease, and graft versus host disease. If target proteins or T cell epitopes responsible for at least a part of said immune reaction are known, it is possible to co-administer said target proteins or T cell epitopes with the TCP of the present invention, e.g., in covalently linked form, such as in the form of a fusion protein. However, that is not required.

At the time of administration, the immune reaction can be present in the subject (such as in an autoimmune or allergic disorder present in the patient, or an immune response to a therapeutic agent, e.g., a substitute therapeutic protein), or the immune reaction can be likely in the future, if the subject is not treated (e.g., an immune reaction in response to an agent to be administered in the future, e.g., a therapeutic agent such as a substitute therapeutic protein). If the agent against which the immune reaction is directed is known, then the TCP can be co-administered with the agent against which the immune reaction is directed. For example, the TCP may be co-administered with an allergen or with the target protein or target epitope of an autoimmmune response, e.g., of an antibody.

Due to the presence of tregitopes, the TCP has immunomodulatory and immunosuppressive properties which can be beneficially exploited already by using the TCP, e.g., as a stand-alone therapeutic agent, in particular, wherein the agent is not co-administered with another active agent. The protein of the present invention may thus be used as an immunosuppressant or immunomodulator. For example, it can be suitably applied for the prevention or treatment of disorders described herein, including autoimmune related disorders, allergy, viral infection, or transplantation-related immune reactions or disorders.

Therefore, the TCP may be for use in administration as stand-alone therapeutic, as defined herein. However, the term “stand-alone” therapeutic does not exclude that the TCP is co-administered with other drugs useful for treating a certain disorder. For stand-alone applications, TCP or the TCP composition does not comprise an agent such as a fused protein or peptide, against which undesired immune response is to be modulated, like an allergen. Preferably, for such stand-alone applications the TCP is administered as a dimer or multimer.

The TCP of the present invention in the form of a stand-alone therapeutic preferably is for use in indications that are known to be advantageously treated with plasma-derived intravenous immunoglobulin G (IVIG) or with subcutaneous plasma-derived immunoglobulin G. For example, the TCP of the present invention as a stand-alone therapeutic, in particular, the protein consisting of or essentially consisting of the dimerized or multimerized TCP, as defined herein, may be advantageously applied in the treatment of allergy, autoimmune diseases such as immune thrombocytopenia, Kawasaki disease, and Guillain-Barré syndrome, type I diabetes, Hepatitis, neurological diseases such as multifocal motor neuropathy, stiff person syndrome, multiple sclerosis, and myasthenia gravis, myositis, chronic inflammatory demyelinating polyneuropathy, thrombotic thrombocytopenic purpura (TTP), systemic lupus erythematosus, Graves' disease, autoimmune thyroiditis, host versus graft disease, graft versus host disease, and chronic pain. In this embodiment, it is not required that the target of the immune response that is to be modulated by the treatment be known.

Alternatively, the TCP for use in modulating an immune response in a subject, for suppressing an immune response or inducing tolerance to be co-administrated with another agent. In that case, said immune response typically is an immune response to an agent with which the TCP is co-administered. As described herein, in one embodiment, the agent and the TCP are not linked. Alternatively, the pharmaceutical composition may be for use in suppression or inhibition of an undesired immune response against an agent non-covalently or covalently linked to the TCP, preferably, covalently linked to the TCP, e.g., in the form of a fusion protein. If the agent of interest is covalently or non-covalently linked to an agent of interest, the TCP is even better suited to confer its tolerance inducing properties than in case of simple co-administration without linkage.

### LIST OF EMBODIMENTS

In a first embodiment (embodiment 1), the present invention provides a tregitope carrying polypeptide (TCP) comprising an amino acid sequence having at least 85% sequence identity with amino acids 135 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity.

In a 2^{nd} embodiment, in the TCP of embodiment 1, the sequence identity with amino acids 135 to 330 of SEQ ID NO: 1 is at least 90%. In a 3^{rd} embodiment, in the TCP of embodiment 1, the sequence identity with amino acids 135 to 330 of SEQ ID NO: 1 is at least 95%. In a 4^{th} embodiment, in the TCP of embodiment 1, the sequence identity with amino acids 135 to 330 of SEQ ID NO: 1 is at least 99%. In a 5^{th} embodiment, in the TCP of embodiment 1, the sequence identity with amino acids 135 to 330 of SEQ ID NO: 1 is 100%.

In a 6^{th} embodiment, the present invention provides a TCP, which may be a TCP of any of embodiments 1-5, comprising an amino acid sequence having at least 85% sequence identity with amino acids 114 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity.

In a r embodiment, in the TCP of embodiment 6, the sequence identity with amino acids 114 to 330 of SEQ ID NO: 1 is at least 90%. In an 8^{th} embodiment, in the TCP of embodiment 6, the sequence identity with amino acids 114 to 330 of SEQ ID NO: 1 is at least 95%. In a 9^{th} embodiment, in the TCP of embodiment 6, the sequence identity with amino acids 114 to 330 of SEQ ID NO: 1 is at least 99%. In a 10^{th} embodiment, in the TCP of embodiment 6, the sequence identity with amino acids 114 to 330 of SEQ ID NO: 1 is 100%.

In an 11^{th} embodiment, the present invention provides a TCP, which may be a TCP of any of embodiments 1-10, comprising an amino acid sequence having at least 85% sequence identity with amino acids 104 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity.

In a 12^{th} embodiment, in the TCP of embodiment 11, the sequence identity with amino acids 104 to 330 of SEQ ID NO: 1 is at least 90%. In a 13^{th} embodiment, in the TCP of embodiment 11, the sequence identity with amino acids 104 to 330 of SEQ ID NO: 1 is at least 95%. In a 14^{th} embodiment, in the TCP of embodiment 11, the sequence identity with amino acids 104 to 330 of SEQ ID NO: 1 is at least 99%. In a 15^{th} embodiment, in the TCP of embodiment 11, the sequence identity with amino acids 104 to 330 of SEQ ID NO: 1 is 100%.

In a 16^{th} embodiment, the present invention provides a TCP, which may be a TCP of any of embodiments 1-15, comprising an amino acid sequence having at least 85% sequence identity with amino acids 1 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity.

In a 17th embodiment, in the TCP of embodiment 16, the sequence identity with amino acids 1 to 330 of SEQ ID NO: 1 is at least 90%. In an 18^{th} embodiment, in the TCP of embodiment 16, the sequence identity with amino acids 1 to 330 of SEQ ID NO: 1 is at least 95%. In a 19^{th} embodiment, in the TCP of embodiment 16, the sequence identity with amino acids 1 to 330 of SEQ ID NO: 1 is at least 99%. In a 20^{th} embodiment, in the TCP of embodiment 16, the sequence identity with amino acids 1 to 330 of SEQ ID NO: 1 is 100%.

In a 21^{st} embodiment, the present invention provides a TCP, which may be a TCP of any of embodiments 1-20, comprising a contiguous sequence of at least 190 amino acids having at least 50%, preferably, at least 60% sequence identity to amino acids No. 135-330 of SEQ ID NO: 1, wherein said TCP comprises at least two regulatory T cell activating epitopes which are heterologous to said Fc-part chain, wherein said protein optionally does not comprise the V_{H} domain and/or the C_{H}1 domain of an antibody. In a 22^{nd} embodiment, at least one, optionally, at least two of the tregitopes of the TCP of embodiment 21 is/are located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1.

In embodiments 21 and 22, the sequences of the frames are taken into account for determination of sequence identity, which leads to the lower sequence identity compared to, e.g., embodiment 1.

In a 23^{rd} embodiment, the TCP of any of embodiments 1-22 comprises at least two heterologous tregitopes, preferably at least three, optionally, four. In a 24^{th} embodiment, the TCP of any of embodiments 1-23 comprises two to four tregitopes.

In a 25^{th} embodiment, in the TCP of any of embodiments 1-24, a first heterologous tregitope is located in one of frames A, B, or C, and wherein at least a second tregitope is located in a different frame of frames A, B, C, or C-terminal to the amino acid sequence having at least 85% sequence identity with amino acids 135 to 330 of SEQ ID NO: 1, optionally linked to said sequence via a linker of 3-18 amino acids.

In a 26^{th} embodiment, in the TCP of any of embodiments 1-25, at least one heterologous tregitope is located in sequence frame A. In a 27th embodiment, in the TCP of any of embodiments 1-26, at least one heterologous tregitope is located in sequence frame B. In a 2e embodiment, in the TCP of any of embodiments 1-27, at least one heterologous tregitope is located in sequence frame C. In a 29^{th} embodiment, in the TCP of any of embodiments 1-28, at least one heterologous tregitope is located in sequence frames A and B. In a 30^{th} embodiment, in the TCP of any of embodiments 1-29, at least one heterologous tregitope is located in each of sequence frames A and C. In a 31^{st} embodiment, in the TCP of any of embodiments 1-30, at least one heterologous tregitope is located in each of sequence frames B and C. In a 32^{nd} embodiment, in the TCP of any of embodiments 1-31, at least one heterologous tregitope is located in sequence frames B or C.

In a 33^{rd} embodiment, in the TCP of any of embodiments 1-32,
(a) if sequence frame A contains no heterologous tregitope, said frame A has at least 85% sequence identity with positions 168 to 203 of SEQ ID NO: 1, and
(b) if sequence frame B contains no heterologous tregitope, said frame B has at least 85% sequence identity with positions 272 to 307 of SEQ ID NO: 1, and
(c) if sequence frame C contains no heterologous tregitope, said frame C has at least 85% sequence identity with positions 212 to 249 of SEQ ID NO: 1.

In a 34^{th} embodiment, the TCP of any of embodiments 1-33 comprises at least one heterologous tregitope C-terminal to the amino acid sequence having at least 85% sequence identity with amino acids 135 to 330 of SEQ ID NO: 1. In a 35^{th} embodiment, in the TCP of embodiment 34, the heterologous tregitope is directly C-terminal to said amino acid sequence. In a 36^{th} embodiment, in the TCP of embodiment 34, the heterologous tregitope is linked to said sequence via a linker of 3-18 amino acids. In a 37^{th} embodiment, in the TCP of embodiment 36, the linker is selected from the group consisting of a GS linker or a linker of any of SEQ ID NO: 107, 108, 109 or 110. In a 38^{th} embodiment, in the TCP of any of embodiments 34-37, the heterologous tregitope C-terminal to the amino acid sequence having at least 85% sequence identity with amino acids 135 to 330 of SEQ ID NO: 1 is selected from the group consisting of Treg134, Treg088x and Treg088. In a 39^{th} embodiment, in the TCP of any of embodiments 34 and 36-37, the heterologous tregitope C-terminal to the amino acid sequence having at least 85% sequence identity with amino acids 135 to 330 of SEQ ID NO: 1 is Treg029B, and the linker has SEQ ID NO: 108.

In a 40^{th} embodiment, in the TCP of any of embodiments 34-39, there is a heterologous tregitope at the C-Terminus of the TCP, optionally, linked to said sequence via a linker of 3-18 amino acids. Alternatively, in a 41^{st} embodiment, in the TCP of any of embodiments 34-39, the heterologous tregitope C-terminal to the amino acid sequence having at least 85% sequence identity with amino acids 135 to 330 of SEQ ID NO: 1 is not at the C-terminus of the TCP, and, preferably, the TCP is a fusion protein.

In a 42^{nd} embodiment, in the TCP of any of the preceding embodiments, the at least one heterologous tregitope substitutes a sequence of amino acids 135 to 330 of SEQ ID NO: 1 having the same length as said tregitope or having the length of the tregitope plus or minus one or two amino acids, wherein, preferably, the at least one heterologous tregitope substitutes a sequence of amino acids 135 to 330 of SEQ ID NO: 1 having the same length as said tregitope.

In a 43^{rd} embodiment, in the TCP of any of the preceding embodiments, at least one heterologous tregitope is selected from the group comprising:

| SEQ ID NO: 10 | |
|---|---|
| (Treg289): | EEQYQSTYRVVSVLTVLHQDW, |
| SEQ ID NO: 7 | |
| (Treg084): | GTDFTLTISSLQPED, |
| SEQ ID NO: 2 | |
| (Treg009A): | GGLVQPGGSLRLSCAASGFTF, |
| SEQ ID NO: 9 | |
| (Treg088x): | KTLYLQMNSLRAEDTAKHYCA, |
| SEQ ID NO: 8 | |
| (Treg134): | LNNFYPREAKVQWKVDNALQSGNS, |
| SEQ ID NO: 3 | |
| (Treg029B): | MHWVRQAPGKGLEWV, |
| SEQ ID NO: 4 | |
| (Treg088): | NTLYLQMNSLRAEDTAVYYCA, |
| SEQ ID NO: 5 | |
| (Treg167): | PAVLQSSGLYSLSSVVTVPSSSLGTQ, |
| SEQ ID NO: 6 | |
| (Treg289n- | EEQYNSTYRVVSVLTVLHQDW, |
| native): | |
| SEQ ID NO: 11 | |
| (trimmed | VQPGGSLRLSCAASG, |
| Treg009A): | |
| SEQ ID NO: 12 | |
| (trimmed | WVRQAPGKGL, |
| Treg029B-v1): | |
| SEQ ID NO: 13 | |
| (trimmed | VRQAPGKGL, |
| Treg029B-v2): | |
| SEQ ID NO: 14 | |
| (trimmed | YLQMNSLRAEDTAVY, |
| Treg088): | |
| SEQ ID NO: 15 | |
| (trimmed | KTLYLQMNSLRAEDTAKH, |
| Treg088x-v1): | |
| SEQ ID NO: 16 | |
| (trimmed | YLQMNSLRAEDTAKH, |
| Treg088x-v2): | |
| SEQ ID NO: 17 | |
| (trimmed | LQSSGLYSLSSVVTVPSSSL, |
| Treg167): | |
| SEQ ID NO: 18 | |
| (trimmed | YNSTYRVVSVLTVLH, |
| Treg289n): | |
| SEQ ID NO: 19 | |
| (trimmed | YQSTYRVVSVLTVLH, |
| Treg289): | |
| SEQ ID NO: 20 | |
| (trimmed | FTLTISSLQ, |
| Treg084): | |
| and | |
| SEQ ID NO: 21 | |
| (trimmed | FYPREAKVQWKVDNALQS, |
| Treg134): | |

wherein, optionally, all tregitopes are selected from said group.

In a 44^{th} embodiment, in the TCP of any of the preceding embodiments, the tregitope is selected from the group consisting of SEQ ID NO: 10, 7, 2, 9, and 8. In a 45^{th} embodiment, in the TCP of any of embodiments 1-44, at least one heterologous tregitope has SEQ ID NO: 10. In a 46^{th} embodiment, in the TCP of any of embodiments 1-44, at least one heterologous tregitope has SEQ ID NO: 7. In a 47^{th} embodiment, in the TCP of any of embodiments 1-44, at least one heterologous tregitope has SEQ ID NO: 2. In a 48^{th} embodiment, in the TCP of any of embodiments 1-44, at least one heterologous tregitope has SEQ ID NO: 9. In a 49th embodiment, in the TCP of any of embodiments 1-44, at least one heterologous tregitope has SEQ ID NO: 8.

In a 50^{th} embodiment, in the TCP of any of embodiments 1-44, at least one heterologous tregitope has SEQ ID NO: 3. In a 51^{st} embodiment, in the TCP of any of embodiments 1-44, at least one heterologous tregitope has SEQ ID NO: 4. In a 52^{nd} embodiment, in the TCP of any of embodiments 1-44, at least one heterologous tregitope has SEQ ID NO: 5. In a 53^{1d} embodiment, in the TCP of any of embodiments 1-44, at least one heterologous tregitope has SEQ ID NO: 6. In a 54^{th} embodiment, in the TCP of any of embodiments 1-44, at least one heterologous tregitope has SEQ ID NO: 11. In a 55^{th} embodiment, in the TCP of any of embodiments 1-44, at least one heterologous tregitope has SEQ ID NO: 12. In a 56^{th} embodiment, in the TCP of any of embodiments 1-44, at least one heterologous tregitope has SEQ ID NO: 13. In a 57^{th} embodiment, in the TCP of any of embodiments 1-44, at least one heterologous tregitope has SEQ ID NO: 14. In a 58^{th} embodiment, in the TCP of any of embodiments 1-44, at least one heterologous tregitope has SEQ ID NO: 15. In a 59^{th} embodiment, in the TCP of any of embodiments 1-44, at least one heterologous tregitope has SEQ ID NO: 16. In a 60^{th} embodiment, in the TCP of any of embodiments 1-44, at least one heterologous tregitope has SEQ ID NO: 17. In a 61^{st} embodiment, in the TCP of any of embodiments 1-44, at least one heterologous tregitope has SEQ ID NO: 18. In a 62^{nd} embodiment, in the TCP of any of embodiments 1-44, at least one heterologous tregitope has SEQ ID NO: 19. In a 63^{rd} embodiment, in the TCP of any of embodiments 1-44, at least one heterologous tregitope has SEQ ID NO: 20. In a 64^{th} embodiment, in the TCP of any of embodiments 1-44, at least one heterologous tregitope has SEQ ID NO: 21.

In a 65^{th} embodiment, in the TCP of any of embodiments 1-65, all tregitopes in one TCP monomer have different sequences. In a 66^{th} embodiment, in the TCP of any of embodiments 1-65, all tregitopes in one TCP monomer have the same sequences.

In a 67^{th} embodiment, the present invention provides a TCP of any of embodiments 1-66, wherein sequence frame A corresponds to positions 170 to 203 of SEQ ID NO: 1, preferably, to positions 173 to 203 of SEQ ID NO: 1. In a 68^{th} embodiment, the present invention provides a TCP of any of embodiments 1-67, wherein sequence frame B corresponds to positions 275 to 306 of SEQ ID NO: 1, preferably, to positions 277 to 304 of SEQ ID NO: 1. In a 69^{th} embodiment, the present invention provides a TCP of any of embodiments 1-68, wherein sequence frame C corresponds to positions 212 to 249 of SEQ ID NO: 1, preferably, to positions 217 to 248 of SEQ ID NO: 1.

In a 70^{th} embodiment, in the TCP according to any of embodiments 1-69,
(a) frame A comprises the tregitope of SEQ ID NO: 10 (Treg289) at position 176 to 196 (i.e., at the position corresponding to the respective position of SEQ ID NO: 1), SEQ ID NO: 5 (Treg167) at position 174 to 199, SEQ ID NO: 2 (Treg009A) at position 180 to 200, SEQ ID NO: 3 (Treg029B) at position 178 to 192, SEQ ID NO: 7 (Treg084) at position 186 to 200, SEQ ID NO: 8 (Treg134) at position 179 to 202, or SEQ ID NO: 15 (trimmed Treg088x-v1) at position 173 to 190; and/or
(b) frame B comprises the tregitope of SEQ ID NO: 10 (Treg289) at position 280 to 300, SEQ ID NO: 5 (Treg167) at position 278 to 303, SEQ ID NO: 2 (Treg009A) at position 278 to 298, SEQ ID NO: 3 (Treg029B) at position 287 to 301, SEQ ID NO: 7 (Treg084) at position 284 to 298, SEQ ID NO: 8 (Treg134) at position 277 to 300, or SEQ ID NO: 15 (trimmed Treg088x-v1) at position 287 to 304; and/or
(c) frame C comprises the tregitope of SEQ ID NO: 10 (Treg289) at position 225 to 245 (or at the position corresponding to the respective position of SEQ ID NO: 1), SEQ ID NO: 5 (Treg167) at position 223 to 248, SEQ ID NO: 2 (Treg009A) at position 223 to 243, SEQ ID NO: 3 (Treg029B) at position 223 to 237, SEQ ID NO: 7 (Treg084) at position 224 to 238, SEQ ID NO: 8 (Treg134) at position 222 to 245, or SEQ ID NO: 15 (trimmed Treg088x-v1) at position 217 to 234; and/or
(d) least one heterologous tregitope located C-terminal to the amino acid sequence having at least 85% sequence identity with amino acids 135 to 330 of SEQ ID NO: 1 has SEQ ID NO: 8 (Treg134), SEQ ID NO: 14 (trimmed Treg088) or SEQ ID NO: 9 (Treg088x), wherein said tregitope is optionally linked to said sequence having at least 85% sequence identity with amino acids 135 to 330 of SEQ ID NO: 1 via a linker of 3-18 amino acids such as a GS linker or a linker of any of SEQ ID NO: 107-110.

In a 71^{st} embodiment, the TCP according to any of embodiments 1-70, is
(I) a TCP comprising
   (a) a tregitope according to SEQ ID NO: 2 (Treg009A) located in frame A, and
   (b) a tregitope according to SEQ ID NO: 2 (Treg009A) located frame B, and
   (c) a tregitope according to SEQ ID NO: 7 (Treg084) located in frame C, and
   (d) a tregitope according to SEQ ID NO: 9 (Treg088x) C-terminal to the amino acid sequence having at least 85% sequence identity with amino acids 135 to 330 of SEQ ID NO: 1, optionally linked to said sequence via a linker of 3-18 amino acids such as a GS linker, wherein said tregitope may be located at the C-terminus of the TCP.

In a 72^{nd} embodiment, the TCP according to any of embodiments 1-70 is
(II) a TCP comprising
   (a) a tregitope according to SEQ ID NO: 9 (Treg088x) located in frame located in frame B, and
   (b) a tregitope according to SEQ ID NO: 2 (Treg009A) located in frame located in frame C.

In a 73^{rd} embodiment, the TCP according to any of embodiments 1-70 is
(III) a TCP comprising
   (a) a tregitope according to SEQ ID NO: 10 (Treg289) located in frame B, and
   (b) a tregitope according to SEQ ID NO: 9 (Treg088x)C-terminal to the amino acid sequence having at least 85% sequence identity with amino acids 135 to 330 of SEQ ID NO: 1, optionally linked to said sequence via a linker of 3-18 amino acids such as a GS linker, wherein said tregitope may be located at the C-terminus of the TCP.

In a 74^{th} embodiment, the TCP according to any of embodiments 1-70 is
(IV) a TCP comprising
   (a) a tregitope according to SEQ ID NO: 10 (Treg289) located in frame A, and
   (b) a tregitope according to SEQ ID NO: 7 (Treg084) located in frame C, and
   (c) a tregitope according to SEQ ID NO: 8 (Treg134)C-terminal to the amino acid sequence having at least 85% sequence identity with amino acids 135 to 330 of SEQ ID NO: 1, optionally linked to said sequence via a linker of 3-18 amino acids such as a GS linker, wherein said tregitope may be located at the C-terminus of the TCP.

In a 75^{th} embodiment, the TCP according to any of embodiments 1-70 is
(V) a TCP comprising
   (a) a tregitope according to SEQ ID NO: 10 (Treg289) located in frame A,
   (b) a tregitope according to SEQ ID NO: 8 (Treg134) located in frame B, and
   (c) a tregitope according to SEQ ID NO: 7 (Treg084) located in frame C.

In a 76^{th} embodiment, the TCP according to any of embodiments 1-70 is
(VI) a TCP comprising
   (a) a tregitope according to SEQ ID NO: 10 (Treg289) located in frame A, and
   (b) a tregitope according to SEQ ID NO: 7 (Treg084) located in frame C, and
   (c) a tregitope according to SEQ ID NO: 9 (Treg088x)C-terminal to the amino acid sequence having at least 85% sequence identity with amino acids 135 to 330 of SEQ ID NO: 1, optionally linked to said sequence via a linker of 3-18 amino acids such as a GS linker, wherein said tregitope may be located at the C-terminus of the TCP.

In a 77^{th} embodiment, the TCP according to any of embodiments 1-70 is
(VII) a TCP comprising
   (a) a tregitope according to SEQ ID NO: 7 (Treg084) located in frame C, and
   (b) a tregitope according to SEQ ID NO: 8 (Treg134)C-terminal to the amino acid sequence having at least 85% sequence identity with amino acids 135 to 330 of SEQ ID NO: 1, optionally linked to said sequence via a linker of 3-18 amino acids such as a GS linker, wherein said tregitope may be located at the C-terminus of the TCP.

In a 7e embodiment, the TCP according to any of embodiments 1-70 comprises an amino acid sequence selected from the group consisting of SEQ ID NOs: 23 to 44 and 46 to 58 and 111.

In a 79^{th} embodiment, the invention provides a TCP according to any of embodiments 1-78,

wherein the TCP comprises at least a part that enables dimer formation, optionally, the complete hinge region of an immunoglobulin.

In an 80^{th} embodiment, the invention provides a TCP according to any of embodiments 1-79,

wherein the TCP comprises from 195 to 350 amino acids.

In an 81^{st} embodiment, the TCP according to embodiment 80 essentially consists of the amino acid sequence having at least 85% sequence identity with amino acids 135 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,

wherein sequence frames A, B, and C are not taken into account for determining the sequence identity.

In an 82^{nd} embodiment, the invention provides a TCP according to any of embodiments 1-81, wherein said TCP does not comprise the V_{H} domain and/or the C_{H}1 domain of an antibody.

In an 83^{rd} embodiment, the invention provides a TCP according to any of embodiments 1-79, wherein the TCP comprises further immunoglobulin superfamily domains, wherein, preferably, the TCP further comprises at least a VH domain and CH1 domain of an antibody, preferably, an antigen-binding part of an antibody.

In an 84^{th} embodiment, the TCP according to any of embodiments 1-80 and 81-83 further comprises a CH3 domain of IgA, and, optionally, a joining region of IgA.

In an 85^{th} embodiment, the TCP according to any of embodiments 1-80 and 82-83 further comprises a CH3 and CH4 domain of IgM.

In an 86^{th} embodiment, the TCP according to any of embodiments 1-85 further comprises an affinity tag selected from the group comprising albumin or an albumin-binding domain. In an 87^{th} embodiment, the TCP according to any of embodiments 1-86 further comprises a linker, e.g., a GS linker or a linker of any of SEQ ID NO: 107-110. In an 88^{th} embodiment, the TCP according to any of embodiments 1-87 further comprises a signal peptide, e.g., having SEQ ID NO: 22.

In an 89^{th} embodiment, the TCP according to any of embodiments 1-88 forms a multimer comprising at least two, three, four, five, six, or more TCP monomers. In a 90^{th} embodiment, the TCP of embodiment 89 forms a dimer comprising at least two TCP monomers according to any one of embodiment 1-88. In a 91^{st} embodiment, in the TCP of embodiment 90, said TCP monomers are covalently bound via at least one disulfide bridge, wherein, optionally, said TCP monomers are covalently linked via an at least partial immunoglobulin hinge region. In a 92^{nd} embodiment, in the TCP of embodiment 91, the partial hinge region has at least 85%, preferably, at least 90%, at least 95% or 100% sequence identity to amino acids 104-113 of SEQ ID NO: 1. In a 93^{rd} embodiment, in the TCP of any of embodiments 91 or 92, the hinge region has at least 85%, preferably, at least 90%, at least 95% or 100% sequence identity to amino acids 99-113 of SEQ ID NO: 1.

In a 94^{th} embodiment, the TCP according to any of embodiments 89-93 consists of TCP monomers according to any of embodiments 80-82.

In a 95^{th} embodiment, the TCP according to any of embodiments 89-93 comprises at least one, preferably, two TCP monomers according to any of embodiments 83-85.

In a 96^{th} embodiment, the TCP according to any of embodiments 1-80, 82-93 and 95 is covalently or non-covalently linked to an agent, wherein the agent preferably is an agent against which an undesired immune reaction is to be suppressed and/or immunogenic tolerance is to be conferred. In a 97th embodiment, in the TCP of embodiment 96, the TCP is covalently linked to said agent. In a 98^{th} embodiment, in the TCP of embodiment 96, the TCP is non-covalently linked to said agent.

In a 99^{th} embodiment, in the TCP of any of embodiments 96-98, said agent is an allergen. In a 100^{th} embodiment, in the TCP of any of embodiments 96-98, said agent is an intolerance inducing agent. In a 101^{st} embodiment, in the TCP of any of embodiments 96-98, said agent is a target protein of an autoimmune response, e.g., of an autoantibody. In a 102^{nd} embodiment, in the TCP of any of embodiments 96-99, said agent is a target epitope of an autoimmune response, e.g., of an autoantibody. It may also be a T-cell epitope that is a target epitope of an autoimmune response. In a 103^{rd} embodiment, in the TCP of any of embodiments 96-99, said agent is a therapeutic agent. In a 104^{th} embodiment, in the TCP of any of embodiments 96-103, said TCP and said agent form a fusion protein.

In a 105^{th} embodiment, the invention provides a nucleic acid encoding the TCP according to any one of embodiments 1-104. In a 106^{th} embodiment, the nucleic acid of embodiment 105 is an expression vector suitable for expressing the TCP in an prokaryotic or eukaryotic host cell and/or a vector for homologous recombination in a prokaryotic or eukaryotic host cell, wherein the host cell preferably is an eukaryotic host cell.

In a 107^{th} embodiment, the invention provides a method of manufacturing a nucleic acid encoding a TCP encoding nucleic acid, preferably the nucleic acid of any of embodiments 105-106, comprising the steps of
(a) providing a nucleic acid sequence encoding a immunoglobulin Fc-part chain,
(b) introducing nucleic acid sequences of one or more heterologous tregitopes into the nucleic acid sequence of step (a) at a position corresponding to one or more of frames A, B, or C of the immunoglobulin Fc-part chain according to SEQ ID NO: 1 as defined herein,
(c) generating a nucleic acid having the sequence of step (b).

In a 108^{th} embodiment, the invention provides an eukaryotic or prokaryotic, preferably, eukaryotic host cell, comprising the nucleic acid of any of embodiments 105-106, wherein, optionally, the host cell is suitable for expressing the TCP.

In a 109^{th} embodiment, the invention provides a method of manufacturing a TCP, comprising steps of
(a) cultivating the host cell according to embodiment 108 under conditions suitable for expression of the TCP;
(b) harvesting the cell or medium comprising the TCP expressed in step (a);
(c) isolating said TCP;
(d) optionally, formulating the TCP of step (c) in a pharmaceutically acceptable composition.

In a 110^{th} embodiment, in the method of embodiment 109, step (c) comprises adsorbing the TCP on an affinity material, wherein said affinity material preferably includes a polyclonal antibody to the Fc-part of human Ig, wherein step (c) optionally includes an affinity chromatography.

In a 111^{th} embodiment, the invention provides a transgenic, preferably, non-human animal comprising the nucleic acid according to any one of embodiments 105-106, e.g., a mouse.

In a 112^{th} embodiment, the invention provides a composition comprising a TCP according to any of embodiments 1-104, preferably, according to embodiments 80-82 or 94, wherein said composition further comprises an agent, wherein the agent optionally is an agent against which an undesired immune reaction is to be suppressed and/or immunogenic tolerance is to be conferred. In a 113^{th} embodiment, in the composition of embodiment 112, said agent is a an allergen. In a 114^{th} embodiment, in the composition of embodiment 112, said agent is an intolerance inducing agent. In a 115^{th} embodiment, in the composition of embodiment 112, said agent is a target protein of an autoimmune response, e.g., of an autoantibody. In a 116^{th} embodiment, in the composition of embodiment 112, said agent is a target epitope of an autoimmune response, e.g., of an autoantibody. I may also be a target epitope of a T-cell based autoimmune response. In a 117^{th} embodiment, in the composition of embodiment 112, said agent is a therapeutic agent.

In a 118^{th} embodiment, the invention provides a kit comprising, separately, a TCP according to any of embodiments 1-104, preferably, according to embodiments 80-82 or 94, and an agent, optionally, an agent against which an undesired immune reaction is to be suppressed and/or immunogenic tolerance is to be conferred. In a 119^{th} embodiment, in the kit of embodiment 118, said agent is an allergen. In a 120^{th} embodiment, in the kit of embodiment 118, said agent is an intolerance inducing agent. In a 121^{st} embodiment, in the TCP of embodiment 118, said agent is a target protein of an autoimmune response, e.g., of an autoantibody. In a 122^{nd} embodiment, in the TCP of embodiment 118, said agent is a target epitope of an autoimmune response, e.g., of an autoantibody. It may also be a T cell epitope that is the target epitope of an autoimmune response. In a 123^{rd} embodiment, in the TCP of embodiment 118, said agent is a therapeutic agent.

In a 124th embodiment, the invention provides a pharmaceutical composition comprising the TCP according to anyone of claims 1 to 104, a nucleic acid according to any of embodiments 105-106, or a host cell or embodiment 108, and, optionally, a pharmaceutically acceptable excipient. Preferably, the pharmaceutical composition comprises the TCP.

In a 125^{th} embodiment, the pharmaceutical composition of embodiment 124 comprises a composition of any of embodiments 112-117 or is a kit of any of embodiments 118-123.

In a 126^{th} embodiment, the invention provides the pharmaceutical composition according to any of embodiments 124-125 for use in modulating an immune response in a subject. In a 127^{th} embodiment, the pharmaceutical composition for use of embodiment 126, is for suppressing an immune response or inducing tolerance, wherein, optionally, said immune response is an immune response to an agent with which the TCP is co-administered, e.g., in covalently-linked form. In a 128^{th} embodiment, the pharmaceutical composition for use of any of embodiments 126 or 127 is for use in suppression or inhibition of an undesired immune response against another agent, wherein the TCP is co-administered with said agent. In a 129^{th} embodiment, the pharmaceutical composition for use of embodiment 126 is for use in suppression or inhibition of an undesired immune response against an agent covalently linked to the TCP.

In a 130^{th} embodiment, the invention provides the pharmaceutical composition according to any of embodiments 124-129 for use in the prevention or treatment of an autoimmune related disorder, allergy, viral infection, or transplantation-related immune reaction or disorder, preferably, for use in the treatment of an autoimmune disorder. In a 131^{st} embodiment, the pharmaceutical composition according to embodiment 130 is for use in the prevention an autoimmune related disorder. In a 132^{nd} embodiment, the pharmaceutical composition according to embodiment 130 is for use in treatment an autoimmune related disorder. In a 133^{rd} embodiment, the pharmaceutical composition according to embodiment 130 is for use in the prevention of an allergy. In a 134^{th} embodiment, the pharmaceutical composition according to embodiment 130 is for use in the treatment of an allergy. In a 135^{th} embodiment, the pharmaceutical composition according to embodiment 130 is for use in the treatment a viral infection. In a 136^{th} embodiment, the pharmaceutical composition according to embodiment 130 is for use in the prevention of a transplantation-related immune reaction or disorder. In a 137^{th} embodiment, the pharmaceutical composition according to embodiment 130 is for use in the treatment of a transplantation-related immune reaction or disorder. In a 138^{th} embodiment, the invention provides the pharmaceutical composition according to any of embodiments 124-129 for use in preventing an autoimmune response to a therapeutic protein.

In a 139^{th} embodiment, the invention provides a method for modulating an immune response, preferably, for suppressing an immune response or inducing tolerance, e.g., in vitro, comprising contacting immune cells with a TCP according to anyone of claims 1 to 104, a nucleic acid according to any of embodiments 105-106, or a host cell or embodiment 108, wherein, optionally, said immune response is an immune response to an agent with which the TCP is covalently or non-covalently linked.

In a 140^{th} embodiment, the heterologous tregitope of any of embodiments 1-139 does not occur identically in the same position in an amino acid sequence having at least 85% sequence identity to SEQ ID NO: 1, e.g., it does not occur identically in the same position in a naturally occurring amino acid sequence having at least 85% sequence identity to SEQ ID NO: 1.

### FIGURE LEGENDS

FIG. 1: Ig domain structure according to Kuby, Immunology, Seventh Edition, W. H. Freeman & Co., New York, 2013, with an indication of the origin of tregitope sequences.

FIG. 2: Sequence structure of the constant part of the human IgG heavy chain (P01857; SEQ ID NO: 1) and the preferred Fc-part sub-sequence of positions 104-330 of SEQ ID NO: 1 (SEQ ID NO: 60) as a carrier molecule sequence. Tregitope substitution frames identified by multiple sequence alignment are highlighted.

FIG. 3: The Fc-part sub-sequence (SEQ ID NO: 60) with intramolecular disulfide bonds, domain boundaries, and substitution frames with local residue numbering.

FIG. 4: Excerpt from the ClustaIX alignment of the tregitope sequences Treg289, Treg167, Treg009A, Treg029B, Treg084, and Treg134 (SEQ ID Nos: 10, 5, 2, 3, 7 and 8) with the full carrier molecule sequence (P01857, pos. 150-220 of SEQ ID NO: 1 are shown, SEQ ID NO: 104). Additional alignment of trimmed Treg088x-v1 against P01857, pos. 104-330 was performed and added. The overall alignment with the full carrier molecule sequence defines frame A of 30 residues.

FIG. 5: Excerpt from the ClustaiX alignment of the tregitope sequences Treg289, Treg167, Treg009A, Treg029B, Treg084, and Treg134 (SEQ ID Nos: 10, 5, 2, 3, 7 and 8) with the partial carrier molecule sequence (P01857, pos. 241-310 of SEQ ID NO: 1 are shown, SEQ ID NO: 105). Additionally, an alignment of trimmed Treg088x-v1 (SEQ ID NO: 15) against P01857 was performed and added. The overall alignment with this partial carrier molecule sequence defines frame B of 28 residues.

FIG. 6: Excerpt from the ClustaIX alignment of the tregitope sequences Treg289, Treg167, Treg009A, Treg029B, Treg084, and Treg134 (SEQ ID Nos: 10, 5, 2, 3, 7 and 8) with the partial carrier molecule sequence (P01857, pos. 205-250 of SEQ ID NO: 1 are shown, SEQ ID NO: 106). The flanking cysteines have not been included into the alignment target. Additionally, an alignment of trimmed Treg088x-v1 (SEQ ID NO: 15) against P01857 was performed and added. The overall alignment with this partial carrier molecule sequence defines frame C of 32 residues.

FIG. 7: Predicted binding energies of single-substituted Fc homo-dimers into frame A (left bar, black), frame B (middle bar, grey), or frame C (right bar, light coloured). Covalent contributions from intermolecular disulfide bonds are ignored.

FIG. 8: Predicted binding energies of triple-substituted Fc homo-dimers. 1: tgp0084fa, tgp0167fb, tgp009Afc; 2: tgp0134fa, tgp029Bfb, tgp0167fc; 3: tgp029Bfa, tgp0289fb, tgp0134fc; 4: tgp0167fa, tgp009Afb, tgp0084fc; 5: tgp0289fa, tgp0134fb, tgp0084fc; 6: tgp0084fa, tgp0167fb, tgp009Afc; 7: P01857 Pos. 104-330, unsubstituted carrier (tgp: tregitope, fa: frame A, fb: frame B, fc: frame C. Covalent contributions from intermolecular disulfide bonds are ignored. the single tregitopes are designated as tgp0084 etc.

FIG. 9: Binding Energies of Selected Hetero-Dimers.
1: P01857 #104-330 (glycosylated), unsubstituted carrier
2: P01857 #104-330 with tgp0289fa-tgp0134fb-tgp0084fc and P01857 #104-330 with tgp029B-tgp0289fb-tgp0134fc
3: P01857 #104-330 with tgp0289fa-tgp0134fb-tgp0084fc and P01857 #104-330 with tgp009Afa-tgp029Bfb-tgp0167fc
4: P01857 #104-330 with tgp0084fa-tgp0134fb-tgp029Bfc and P01857 #104-330 with tgp0167fa-tgp0289fb-tgp009Afc
5: P01857 #104-330 with tgp0289fa-tgp0134fb-tgp0084fc and P01857 #104-330 with tgp029Bfa-tgp0167fb-tgp009Afc
6: P01857 #104-330 with tgp029Bfa-tgp0289fb-tgp0134fc and P01857 #104-330 with tgp0289fa-tgp0134fb-tgp0084fc
7: P01857 #104-330 with tgp0134fa-tgp0289fb-tgp0084fc and P01857 #104-330 with tgp0167fa-tgp009Afb-tgp029Bfc
8: P01857 #104-330 with tgp009Afa-tgp0084fb-tgp0289fc and P01857 #104-330 with tgp0134fa-tgp029Bfb-tgp0167fc
9: P01857 #104-330 with tgp0134fa-tgp0167fb-tgp009Afc and P01857 #104-330 with tgp029Bfa-tgp0084fb-tgp0289fc
10: P01857 #104-330 with tgp029Bfa-tgp009Afb-tgp0167fc and P01857 #104-330 with tgp0084fa-tgp0134fb-tgp0289fc
11: P01857 #104-330 with tgp0084fa-tgp0167fb-tgp0134fc and P01857 #104-330 with tgp009Afa-tgp029Bfb-tgp0289fc

FIG. 10: Fc dimer as a tregitope carrying polypeptide molecule in the sense of the protein of the invention. Correlation of total energies of hetero-dimeric complexes [E(A:B)] (lower graph) and monomers [E(A), E(B)] (upper graphs) with binding energies.

FIG. 11: Expression analysis of construct V32 and three direct tregitopes (Dir-Treg). Plasmid DNA carrying sequence information either for construct V32 or Dir-Treg-01-FLAG or Dir-Treg-02-FLAG or Dir-Treg-03-FLAG (SEQ ID NO: 101-103), whereas each Dir-Treg describes three successively cloned tregitope sequences C-terminally followed by a FLAG-Tag, were nucleofected under identical conditions into CAP-T cells and protein expression was performed for 4 days. Cell supernatants were harvested by centrifugation. Construct V32 was diluted 1:10, Dir-Treg-Ox-FLAG supernatants were diluted 1:2 and samples were loaded onto a SDS-PAGE gel. Carboxy-terminal FLAG-BAP Fusion Protein (Sigma-Aldrich, P7457-.1MG) was used in a serial dilution as control. Precision Plus Protein All Blue Standard (Bio-Rad, 161-0373) was used for size identification. Subsequently after the SDS-PAGE run, proteins from the gel were blotted onto a PVDF membrane and fluorescent detection was carried out using anti-FLAG and anti-Fc antibodies. Expression of construct V32 resulted in dramatically higher protein amounts compared to Dir-Treg-Ox-FLAG variants.

FIG. 12: Western Blot analysis of FcTregV1, V3, V13, V14, V20, V23, V32 and V34 and the corresponding unmodified Fc-part (SEQ ID 60). CAP-T cells were transiently transfected with plasmids encoding the respective constructs and 4-days cell culture supernatants were loaded and separated by reduced SDS-PAGE. Western Blot analysis was carried out using an AffiniPure Mouse Anti-Human IgG, Fcγ fragment specific primary antibody and IRDye 800CW Donkey anti-Mouse secondary antibody. All tregitope carrying polypeptides are well expressed and secreted. Protein sizes obtained by appling the Precision Plus Protein All Blue Prestained Protein Standards are indicated.

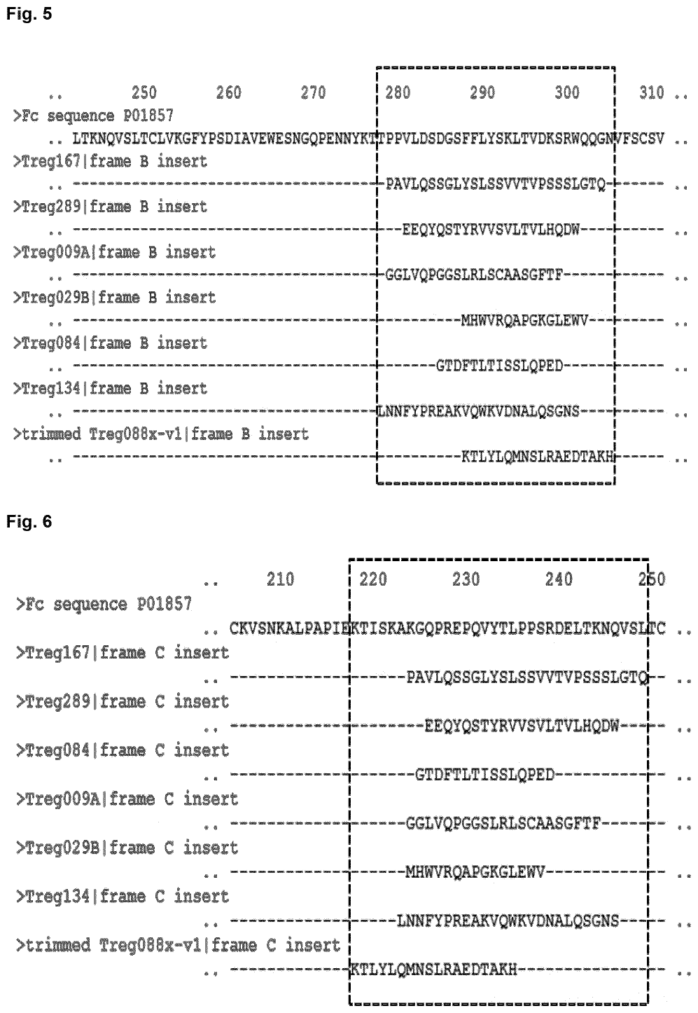

List of Sequences
SEQ ID NO: 1: human wt IgG constant regions
SEQ ID NO: 2: Treg009A
SEQ ID NO: 3: Treg029B
SEQ ID NO: 4: Treg088
SEQ ID NO: 5: Treg167
SEQ ID NO: 6: Treg289n-native
SEQ ID NO: 7: Treg084
SEQ ID NO: 8: Treg134
SEQ ID NO: 9: Treg088x
SEQ ID NO: 10: Treg289
SEQ ID NO: 11: trimmed Treg009A
SEQ ID NO: 12: trimmed Treg029B-v1
SEQ ID NO: 13: trimmed Treg029B-v2
SEQ ID NO: 14: trimmed Treg088
SEQ ID NO: 15: trimmed Treg088x-v1
SEQ ID NO: 16: trimmed Treg088x-v2
SEQ ID NO: 17: trimmed Treg167
SEQ ID NO: 18: trimmed Treg289n
SEQ ID NO: 19: trimmed Treg289
SEQ ID NO: 20: trimmed Treg084
SEQ ID NO: 21: trimmed Treg134
SEQ ID NO: 22: signal peptide
SEQ ID NO: 23-44 and 46-58: FcTregV1-V22 and V24-V36
SEQ ID NO: 59: na encoding signal peptide
SEQ ID NO: 60: Fc-part sub-sequence
SEQ ID NO: 61-96: na encoding FcTregV1-V22 and V24-V36
SEQ ID NO: 97: Dir-Treg01-FLAG
SEQ ID NO: 98: Dir-Treg02-FLAG
SEQ ID NO: 99: Dir-Treg03-FLAG
SEQ ID NO: 100: FLAG sequence
SEQ ID NO: 101: Dir-Treg01-FLAG DNA
SEQ ID NO: 102: Dir-Treg02-FLAG DNA
SEQ ID NO: 103: Dir-Treg03-FLAG DNA
SEQ ID NO: 104: partial sequence of SEQ ID NO: 1 shown in FIG. 4
SEQ ID NO: 105: partial sequence of SEQ ID NO: 1 shown in FIG. 5
SEQ ID NO: 106: partial sequence of SEQ ID NO: 1 shown in FIG. 6
SEQ ID NO: 107: linker1
SEQ ID NO: 108: linker2
SEQ ID NO: 109: linker3
SEQ ID NO: 110: sequence or partial sequence of GS linker
SEQ ID NO: 111: FcTregV32_variant

### Examples

Molecular Modeling Study to Identify a Carrier Platform for Tregitopes

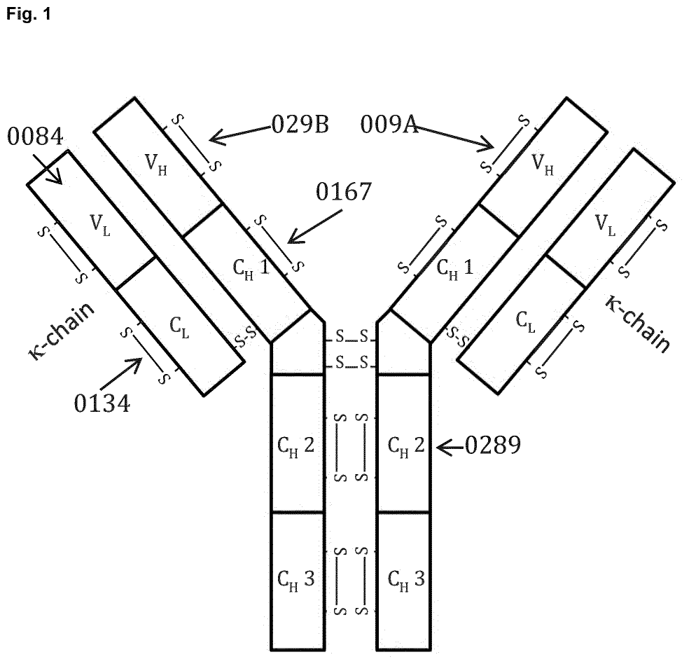

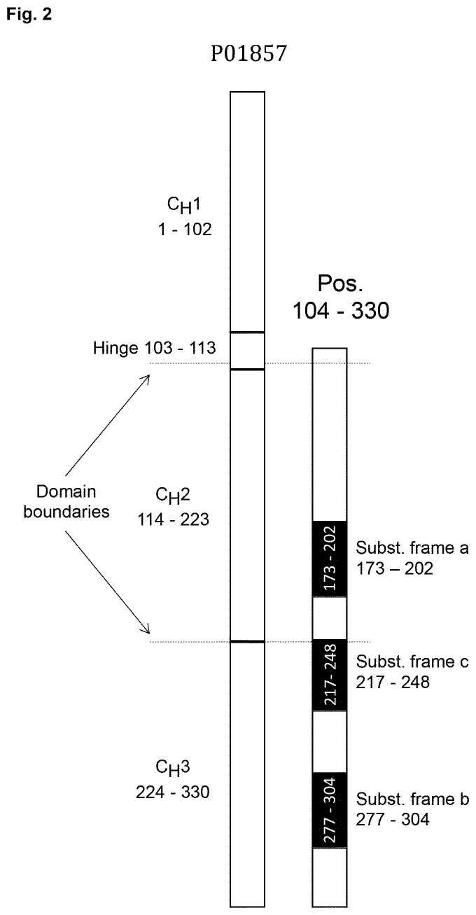

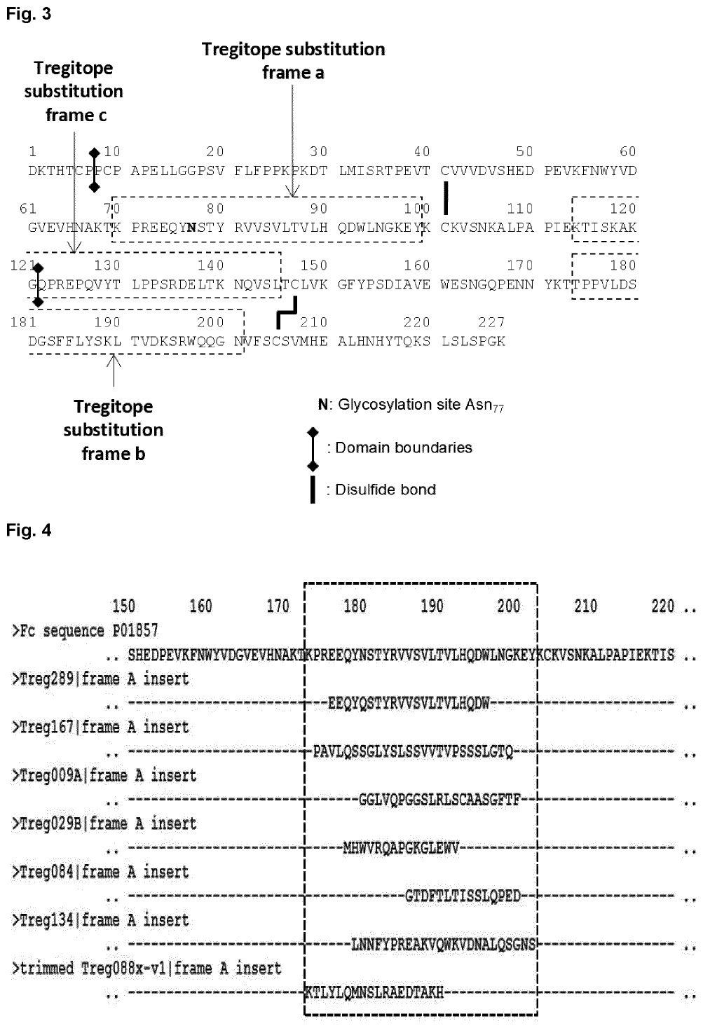

Tregitopes are peptides originally found in the constant region of human and primate type G immunoglobulins (IgGs) that are able to activate regulatory T cells. Recombinant production of these peptides, however, is extremely difficult. In accordance with their natural origin, the Fc-part of human IgG was selected as a cloning framework candidate for a set of different tregitopes (SEQ ID NOs: 2, 3, 5, 6, 7, 8). These sequences were originally derived from different domains of immunoglobulins as shown in FIG. 1. The aim of the present experiment was to identify suitable sequence frames for tregitope cloning and expression. The exact basic sequence used in this study was the UNIPROT sequence P01857 (SEQ ID NO: 1), positions 104 through 330 (FIG. 2), which comprises the CH2 and CH3 domains of the constant region of the heavy chain of human IgG1, starting with part of the hinge region. Sequence details are given in FIG. 3.

Potential substitution frames within the carrier molecule sequence were identified by CLUSTALX multiple sequence alignment. To maintain the epitope character of the tregitopes, an alignment without gaps in the tregitope sequences was generated. This was achieved by using the highest possible gap penalty value (=100). FIGS. 4, 5, and 6 show the alignment of the tregitope sequences with the carrier molecule sequence. Relative to the full carrier molecule sequence, the alignments in frame A (FIG. 4) are obtained. To avoid perturbing the intramolecular disulfide bonds, the full sequence was divided into sections, which do not cover the cysteine residues. Those sections were additionally aligned with the tregitopes. This leads to the alignments shown in FIG. 5 (frame B) and FIG. 6 (frame C). It should be noted that the homology for these other two sections is significantly lower than for the full sequence.

In its biologically active form, the Fc-part is a homo-dimer comprising CH2 and CH3 domains, which is covalently connected by intermolecular disulfide bonds in the hinge region (FIG. 1). A model based on homology to similar sequences was built by a computational method called homology modeling using the YASARA software suite. The energetically most favorable structure was selected as the resulting model structure. Poor convergence and failure to form a dimer predicted by the software were taken as indications for real-world folding problems and interpreted as forecast of instability. Based on the calculated structure models, it could be shown that frame A entirely belongs to domain CH2, while frame B is more or less in the middle of domain CH3. Frame C overlaps with domain CH2 and comprises the domain boundary between CH2 and CH3. As a consequence, frame A was supposed to be the least critical area for tregitope substitution, while frames B and C may have more pronounced impact on the structures of the monomers, of the dimer, and the dimer binding energy.

The fold of the substitution frames was predicted to always comprise a δ-strand and a loop or short helix at either or both ends. In each case, the δ-strand is paired with other strands in the same domain, underlining a close coupling with the other secondary structure elements of the carrier. However, none of the frames is directly involved in intermolecular interactions with another Fc molecule chain. A correctly folded carrier should display a binding energy comparable to the existing carrier structure (P01857). Formation of the intermolecular disulfide bonds in the hinge region can only be expected if a stable dimer structure is formed. It has been shown that the interaction between the CH3 domains is the dominant contribution to this dimer formation. Thus, the CH3-CH3 interaction energy of the model is a useful criterion for a first validation of a predicted structure. For prediction of annealing and minimization, no water molecules were used. Instead, a special force field, the YASARA NOVA force field, which has been parametrized to reproduce crystal structures as close as possible, has turned out to be a reasonable compromise between computing effort and precision for dimerization energy assessment.

Practically, in the case of Fc-part variants (approximately 7500 atoms), a number of 36 cycles of simulated annealing and steepest descent minimization following the homology modeling process, turned out to be a useful strategy to achieve convergence in structures, total energies, monomer energies, and binding energies. Nevertheless, classical force-field based models are limited in their precision and should only be interpreted for comparison of similar structures and the qualitative deduction of trends therefrom. Reference to reliable experimental data has been done in order to increase the reliability of the results.

The following structure variants have been analyzed:
a) Single-tregitope equipped Fc homo-dimers (1 tregitope per Fc molecule for each frame)
b) Triple-tregitope equipped Fc homo-dimers (3 tregitopes per Fc molecule, 1 tregitope in each frame)
c) Sextuple-tregitope equipped Fc hetero-dimers (3 tregitopes per Fc molecule, 1 tregitope in each frame, different tregitopes in each Fc monomer)
d)C-terminal attachment of tregitopes
e) Combinations of a), b), and d)

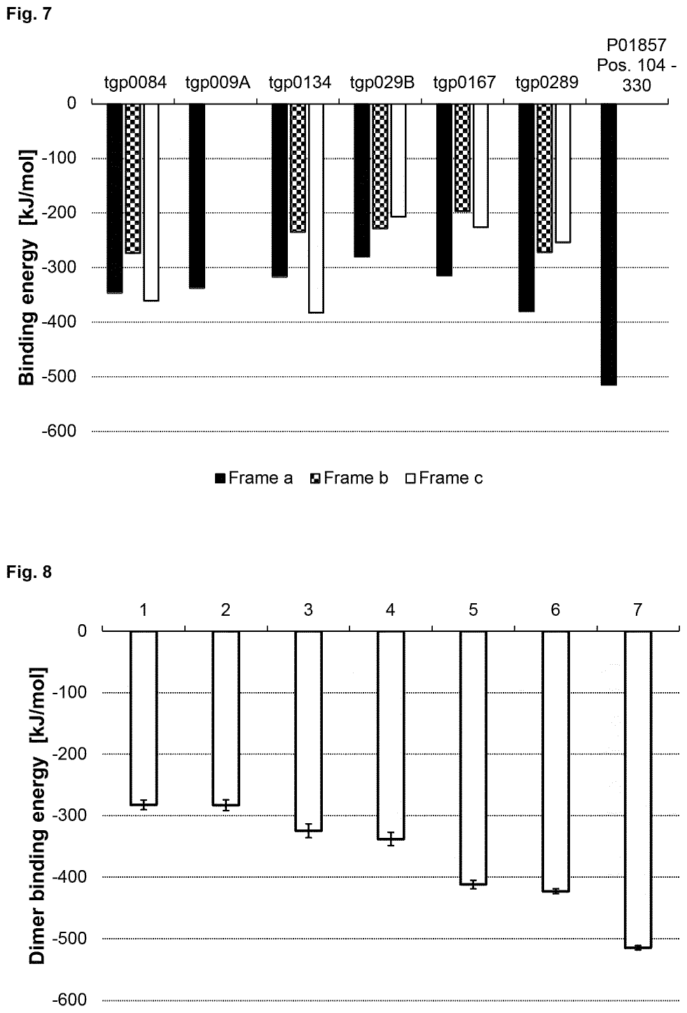

FIG. 7 shows the dimer binding energies of a TCP (for case a). It has to be noted that these and all the other results ignore the covalent contribution from the two disulfide bonds in the hinge region. Their contribution is assumed to be identical for all tregitope insertion variants. As expected, any substitution of the basic sequence with tregitopes leads to a reduction of binding energy. Modified, respectively missing glycosylation are likely to contribute significantly to this difference. It should be noted that none of the variants modeled did have the same glycosylation pattern as the original Fc fragment. The carrier model structure bears the glycosylation pattern from its leading template, the PDB crystal structure 3SGK. This structure is glycosylated at Asn77 of SEQ ID NO: 60 (cf. FIG. 3). This is position Asn180 in SEQ ID NO: 1 (cf. FIG. 4). It is clear that glycosylation could at most be expected with tgp084, tgp0134 and with tgp088x, which also have a Asn in this very position. Insertion of all other tregitopes in frame A leads to a loss of this glycosylation site. From the data, it is not clear to which extent glycosylation affects the dimer binding energy. On the one hand, tgp0289, which differs from the original Fc sequence only by the substitution N180Q (see FIG. 4), is closest to the Fc dimer, however, a difference in binding energy of 135 kJ/mol is surprising. It mainly originates from differences in dihedral and electrostatic energy. In particular, the difference in electrostatic energy is a hint towards solvation phenomena, which are not taken into account explicitly with the force filed used. The difference in binding energy may hence be overestimated by the force field. With the exception of tgp0084 and tgp0134, this also applies to frame C. The diagram also confirms the critical role of frame B, which directly affects the dimer binding energy. Tgp009A should be advantageously introduced into frame A.

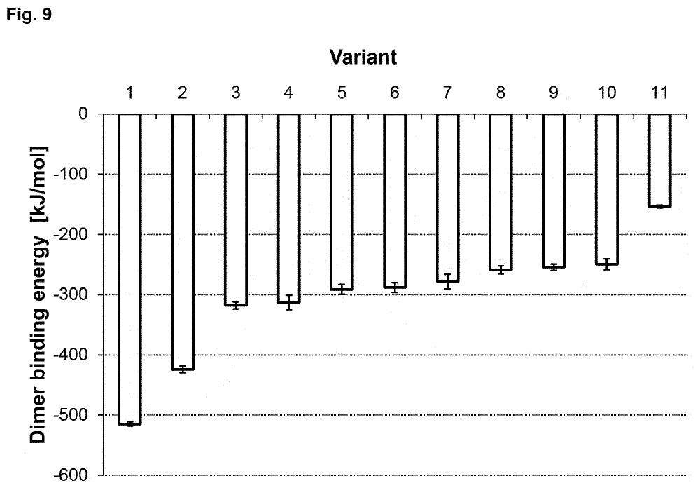

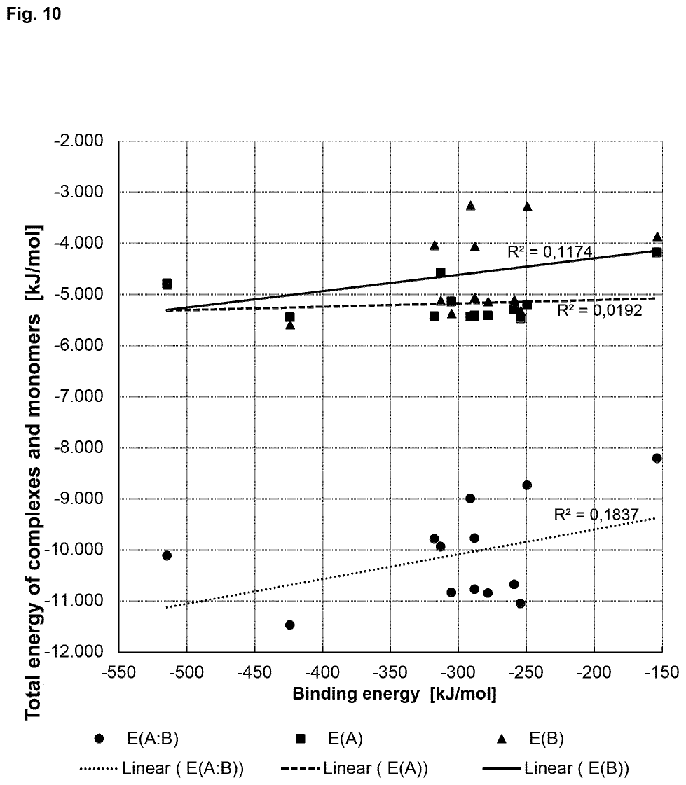

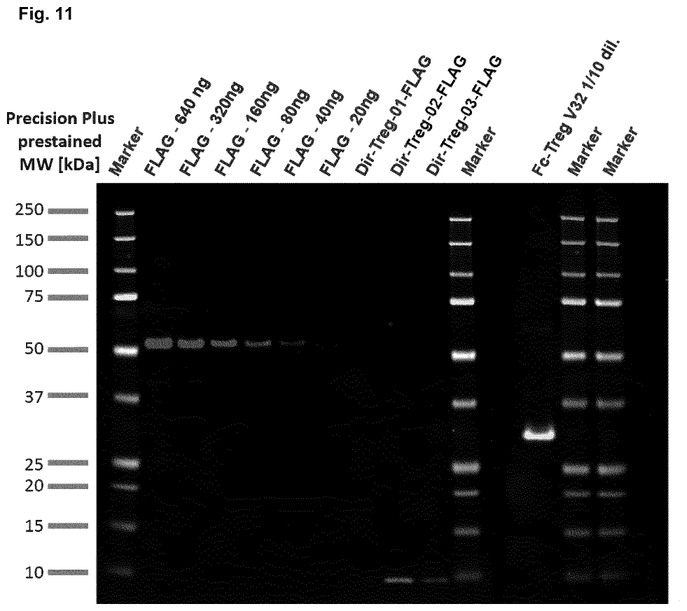

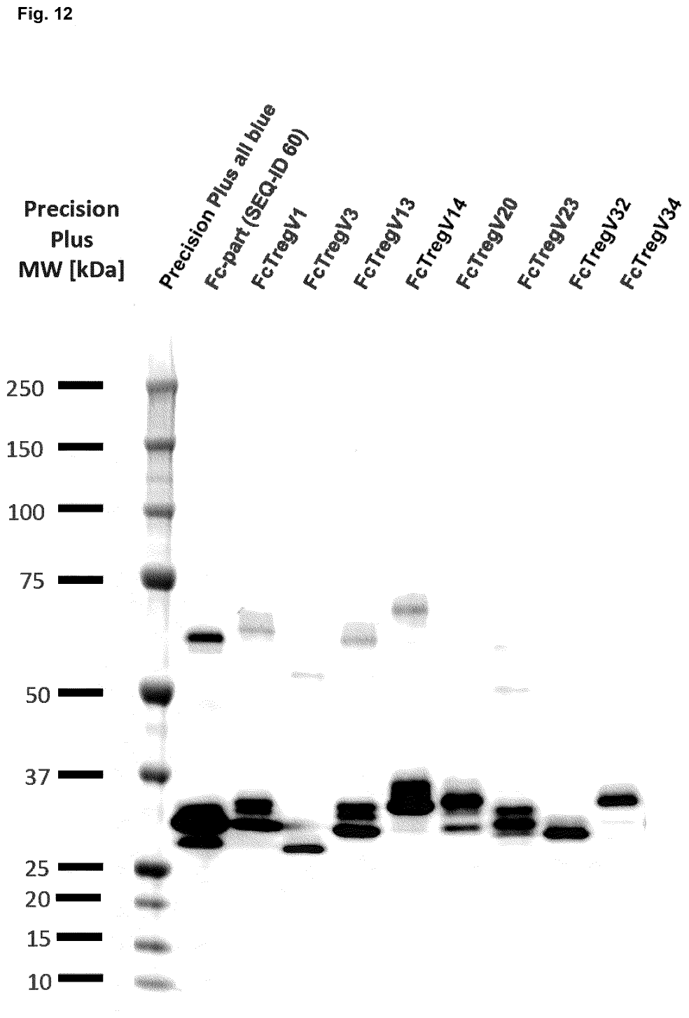

FIG. 8 shows the results for case b), the triple substituted Fc-part variants as homodimers. First of all, there seems to be a certain synergistic effect on the binding energy from the triple substitution of tregitopes, comparing the data with those from FIG. 7. The error bars show the standard deviation (fluctuations) of the binding energy during the optimization process.

Examples of case c) substitutions are given in FIG. 9. These are no longer homo-dimers but hetero-dimers, which do not have any intrinsic symmetry. With the exception of variant 2, all hetero-dimer variants are significantly less stable than the unsubstituted Fc dimer. However, in comparison with the homo-dimers as of FIG. 8, FIG. 9 shows that only variant 11 is significantly less stable than those homo-dimers. In principle, it is possible to generate hetero-dimers containing six different tregitopes experimentally. Variants 7, 8, and 9 could also have at least one glycosylated monomer.

FIG. 10 gives an overview of the energy situation of hetero-dimers. The majority of variants shows binding energies between −250 kJ/mol and −320 kJ/mol with total complex energies between −8740 kJ/mol and −11000 kJ/mol. Exceptions in this representation are variant 11, as defined in FIG. 9, with a very weak binding energy (−154 kJ/mol binding energy; −8214 kJ/mol total complex energy)), the very stable variant 2 (−424 kJ/mol binding energy; −11481 kJ/mol total complex energy), and the unsubstituted carrier variant 1 (−515 kJ/mol binding energy; −10123 kJ/mol total complex energy). Variant 2 appears to be unique in that there is a fortuitous cancellation of stability problems caused by the tregitopes. A certain indication of consistency is the weak correlation found between total energies and binding energies. Furthermore, as the number of atoms does not differ dramatically between the variants (7200-7600 atoms) the vertical spread of the energies gives the order of magnitude for both, force field precision and entropic contributions.

A preliminary analysis of direct tregitope attachment to the C-terminus of the Fc-parts revealed a destabilization of the respective dimers (data not shown). The reason may be related to the fact that the carboxy group of the C-terminal arginine is not surface accessible, but hidden in the internal of the dimer structure. Any modification, which leads to a change of position of the C-termini may lead to a deformation of the CH3 domain and reduces the dimer binding energy. Three linker versions (SEQ ID NO: 107-109) have been analyzed with three different tregitope constructs without frame B substitution. One of the linkers, linker 3 (SEQ ID NO: 109), has also been used with a truncated Fc molecule missing the normal C-terminal lysine residue. It was found that linker 2 (PTGSG; SEQ ID NO: 108) gives an improvement of the binding energy, which is more pronounced with tgp029B as C-terminal attachment (e.g., variant 3; carrier sequence with tgp009Afa (Treg009A in frame a), no fb (no tregitope in frame B), tgp0084fc (Treg084 in frame C, and tgp029B (Treg029B)C-terminal attachment—Homodimer) than with tgp0289 (variant 1; carrier sequence with tgp009Afa, no fb, tgp0084fc, and tgp0289 C-terminal attachment—Homodimer).

Expression of Different TCPs

36 different expression constructs for TCPs (also designated FcTreg herein), constructs FcTregV1 up to FcTregV22 and FcTregV24-FcTregV36, were prepared. FcTregV23 was also prepared (SEQ ID NO: 45). The amino acid sequences (SEQ ID NO: 23-44 and 46-58) and nucleic acid sequences (SEQ ID NO: 61-82 and 84-96) of the respective TCP variants are provided in the sequence listing of the present disclosure. For secretion of all constructs, a Fc signal peptide was used, e.g., Fc-Signal_AA (SEQ ID NO: 22): METDTLLLWVLLLWVPGSTG.

This signal sequence was encoded by Fc-Signal_DNA
(SEQ ID NO: 59):
ATGGAAACCGACACACTGCTGCTGTGGGTGCTGCTTTTGTGGGTGCCAGG
CAGCACCGGC.

The signal sequence was added at 5′ terminus of the DNA respectively N-terminus of the protein. The signal peptide is cleaved off during transport and secretion of the protein.

For analysis of the expression and dimer formation, HEK293F cells and CAP-T cells have been used for transient expression of the constructs. CAP-T cells are an immortalized cell line based on primary human amniocytes and grow in suspension in PEM medium (Life Technologies) supplemented with 4 mM L-Glu. Compared to CAP Go cells, CAP-T cells additionally express the large T antigen of simian virus 40. The HEK 293-f cell line is derived from the original HEK 293 cell line and is adapted to suspension growth in serum-free medium. Transient transfection was done by electroporation using the commercially available Nucleofector™ system.

During the exponential growth phase of the culture, the CAP-T cells were counted by Cedex XS (Roche Applied Science, Innovatis) and viable cell density and viability were determined. For each nucleofection reaction, 1·10⁷CAP-T cells were harvested by centrifugation (150×g for 5 min). The cells were resuspended in 100 μL complete nucleofector solution SE (Lonza, Switzerland) and mixed with the respective Fc-Treg construct (plasmid encoding the tregitope carrier molecule). The DNA/cell suspension was transferred into a cuvette and the nucleofection was performed using the X001 program on a Nucleofector II. After the pulse, cells were recovered by adding 500 μL prewarmed complete PEM medium (=supplemented with 4 mM L-alanyl-L-glutamine) to the cuvette and gently transferred into 11.5 mL complete PEM medium in a 125 mL shaking flask. The cuvette was washed once with 500 μL fresh medium to recover residual cells. The final cultivation volume was 12.5 ml. Electroporation was similarly performed with 7·10⁶ HEK293-F cells and 7 μg plasmid. After transfection the cells were incubated for 4 days. Cell pellets and the supernatant were subsequently tested by Western Blot. Reference was transfection with Fc monomer. All tested constructs showed expression in the pellet, but differences in secretion were observed. There was a good correlation in between observed expression in HEK293F and CAP-T cells.

Molecules V1, V3, V13, and V14 gave good results in secretion and expression in HEK293F. V7, V9 and V12 resulted also in secretion and expression, although to a slightly lesser extent. The TCP performing best under these aspects were V1, V3, V13 and V14 (V13 and V14 were only tested in CAP-T cells). Further tests with supernatants of V15-V36 in CAP-T cells showed particularly good results for V20, V23, V32 and V34.

Thus, preferred TCP of the invention have the following structure, wherein frames not noted do not comprise a heterologous tregitope:
(a) Treg289 in frame A, Treg084 in frame C, Treg134 C-terminal, e.g., V1
(b) Treg289 in frame A, Treg084 in frame C, Treg134 in frame B, e.g., V3
(c) Treg289 in frame A, Treg084 in frame C, Treg88 C-terminal, e.g., V13
(d) Treg084 in frame C, Treg134 C-terminal, e.g., V14
(e) Treg009A in frame C, Treg088x in frame B, e.g., V20
(f) Treg084 in frame A, e.g., V23
(g) Treg009A in frame A, Treg084 in frame C, Treg009A in frame B, Treg088x C-terminal, e.g., V32
(h) Treg289 in frame B, Treg088x C-terminal, e.g., V34.

| | | | | | | Dimer |
|---|---|---|---|---|---|---|
| Name | Frame A | Frame C | Frame B | C-terminal | Expression | formation |
| Construct V1 | Treg289 | Treg084 | | Treg134 | ++ | ++ |
| Construct V3 | Treg289 | Treg084 | Treg134 | | + | +/− |
| Construct V13 | Treg289 | Treg084 | | Treg088x | ++ | +++ |
| Construct V14 | | Treg084 | | Treg134 | ++ | + |
| Construct V20 | | Treg009A | Treg088x | | ++ | +++ |
| Construct V23 | Treg084 | | | | ++ | ++ |
| Construct V32 | Treg009A | Treg084 | Treg009A | Treg088x | ++ | ++ |
| Construct V34 | | | Treg289 | Treg088x | ++ | ++ |

An exemplary Western Blot of FcTregsV1, V3, V13, V14, V20, V23, V32 and V34 and the corresponding unmodified Fc-part (SEQ ID NO:60) is demonstrated in FIG. 12. Cell culture supernatants of transiently transfected CAP-T cells as described above, were prepared by mixing 20 μL of the respective supernatant with 10 μL NuPage LDS Sample Buffer (4×, Thermo Fisher), 4 μL NuPage Sample Reducing Agent (10×, Thermo Fisher) and 6 μl Aqua Dest. (B. Braun, Germany). Samples were denatured for 10 min at 70° C. and 10 μL of the prepared samples were loaded onto a NuPAGE gradient 4-12% BisTris gel. Precision Plus Protein All Blue Prestained Protein Standards (Bio-Rad) was applied as size marker. Reduced sodium dodecyl sulphate—polyacrylamide gel electrophoresis (SDS-PAGE) was performed at 200V and using MOPS buffer (40 mL NuPAGE MOPS SDS running buffer+760 mL Aqua dest. +500 μL NuPAGE Antioxidant). Subsequent Western Blotting was performed onto a nitrocellulose membrane at 30V for 60 min using a transfer buffer (50 mL NuPAGE Transfer Buffer (20×, Thermo Fisher)+1 mL NuPAGE Antioxidant+100 mL methanol+849 mL Aqua Dest.). In order to block unspecific antibody binding for detection, the membrane was first blocked with Odyssey Blocking Buffer (PBS) at 4° C. over night. Subsequently, AffiniPure Mouse Anti-Human IgG, Fcγ fragment specific (Jackson ImmunoResearch, 209-005-098) was used as primary antibody and incubated for 1 h at room temperature under gentle shaking, diluted 1:100 in a solution prepared by mixing 30 mL blocking buffer+0.05% Tween 20. The secondary antibody IRDye 800CW Donkey anti-Mouse (diluted 1:15000, Li-Cor) was incubated for 1 h at 4° C. Between and after the primary and secondary antibody incubation steps, the membrane was washed four times each with approx. 25 mL of PBS-T (500 mL PBS+0.1% of a 10% Tween 20 solution). After rinsing the membrane twice for 5 min with PBS, it was dried over night protected from light. The membrane was scanned using an Odyssey CLx Imager (Li-Cor) for antibody-marked protein band detection.

Western Blot results based on a reduced SDS-PAGE as shown in FIG. 12 clearly demonstrate the particular good expression and secretion of FcTregV1, V3, V13, V14, V20, V23, V32, and V34. TCPs are mostly visible as monomers in size ranges of approx. 26 to 36 kDa due to reduced conditions during the SDS-PAGE run. Multiple bands within individual FcTreg variants within this size range might be different post-translational modification species (e.g glycosylation) as proteins result from transient transfected pools and not from clonal expression. A small portion of dimerized TCP molecules remained as bands between approx. 55 and 70 kDa are visible.

These results show that it is possible to effectively express and produce tregitopes by integration into an immunoglobulin Fc-part according to the inventive approach.

The molecules V1, V3, V13, V14, V20, V23, V32 and V34 were thus chosen to generate CAP Go basic cell lines stably expressing the recombinant proteins. Transfection of CAP Go cells was carried out as described above for CAP-T cells, but using solution V instead of solution SE and running the transfection program X001 on a Nucleofector II. In addition, selection with blasticidin was started 72 h after nucleofection.

Several attempts to purify the recombinant TCP variants from cell culture supernatants by common affinity purification protocols via protein A/G or Thermo Scientific's FcXL column failed. The TCPs did not properly bind to the resin and were found in the flow through. A specific affinity purification strategy was developed by applying a polyclonal mouse anti-human IgG, Fc-gamma fragment specific antibody which has been shown to bind the recombinant protein variants in Western blot detections. This antibody (AffiniPure polyclonal mouse anti-human IgG, Fc-gamma fragment specific antibody (Jackson ImmunoResearch, Cat 209-005-098)) was used as capturing antibody for affinity chromatography.

The commercially available AffiniPure polyclonal mouse anti-human IgG, Fc-gamma fragment specific antibody (Jackson ImmunoResearch, Cat 209-005-098) was covalently conjugated to NHS-activated Sepharose 4 Fast Flow resin (GE Healthcare, Cat. 17-0906) by applying the following steps:
(a) Anti-human IgG, Fc-gamma fragment specific antibody buffer was exchanged to coupling buffer (0.2 M NaHCO₃, 0.5 M NaCl, pH 8.3).
(b) NHS-activated Sepharose 4 Fast Flow matrix was washed 6 times with 1×matrix volume of 1 mM HCl and once with 1×matrix volume of coupling buffer.
(c) 12.5 mL anti-human IgG, Fc-gamma fragment specific antibody (˜1 mg/mL) was added to 25 ml prepared NHS-activated Sepharose 4 Fast Flow resin. Conjugation was carried out at 2-8° C. overnight using a rotator.
(d) Non-reacted groups of the matrix were blocked by incubation for ˜4 h with 0.1 M Tris-HCl, pH ˜8.5.
(e) Washing of resin was carried out using 0.1 M Tris-HCl buffer, pH 8 to 9, and 0.1 M acetate buffer, 0.5 M NaCl, pH 4 to 5. The washing procedure was: 3×1 matrix volumes Tris buffer followed by 3×1 matrix volumes acetate buffer. This cycle was repeated 3 to 6 times. Finally, the resin was kept in 20% ethanol.
(f) The resin was packed into a XK 16/40 or Tricorn 10/300 column.

For purification of the recombinant protein variants from cell culture supernatants of molecule expression preparations, the cell culture supernatants were firstly adjusted to pH 7.4. The supernatants were loaded onto the prepared affinity column with flow rates of 2-6 ml/min and pressure of 0.15-0.2 MPa. The column was then washed with DPBS (Dulbecco's phosphate-buffered saline). The recombinant protein variants were eluted using 100 mM glycine-HCl, pH 2.7. Flow rates and pressures were identical to the loading step. About 10% of the final fraction volumes was used for neutralization with 1 M Tris-HCl pH 8.8. The recombinant protein variants were rebuffered to PBS (phosphate-buffered saline) and concentrated (˜30×) using Pierce Protein Concentrators (Thermo, Cat: 88535). Optionally, Amicon ultrafiltration filters (Merck, Cat: ufc901024) were used for further concentration.

Bystander Suppression Assay

A bystander suppression assay, based on ex vivo stimulation of PBMC (peripheral blood mononuclear cells) of healthy donors with the corresponding antigen leading to a proliferative response with tetanus toxoid (TT assay), was used to assess the molecule constructs with respect to their inhibitory capacity on proliferation/activation of effector CD4 cells. When selecting donors, consideration was given to obtain an as wide as possible coverage of the human population (covering the main HLA-DR B1 supertypes), covering more than 95% of the allelic variability in the human population. Analysis of the incubated cells was performed with immunostaining with intracellular and cell surface markers and analyzed by flow cytometry. Inhibitory effect of the molecule constructs was observable as diminished proliferation and activation of effector CD4 T cells. Particularly effective molecule constructs were identified by statistical analysis.

The assay was performed by plating 3×10⁵ cells/well in 96-wells plates at day 0, each data point performed in duplicate. All subsequent operations including addition of stimuli, tregitopes, antibodies for immunostaining and flow cytometry set up were done without removing the cells from the plates. Stimulation of the PBMC was carried out at day 1 with 0.5 μg/mL tetanus toxoid (TT) in the presence of either 0, 10, 20, 40 or 80 μg/mL of the TCP constructs. Controls receiving only TCP constructs or only TT, as well as controls receiving none of these were included as well. Readout was carried out at day 7 following effector (proliferation, CD25), memory (CCR7, CD45RA) and regulatory (FoxP3, CD25) T cell markers.

The TCP variant V20 (containing tregitopes 009A and 088x) was tested in PBMC from two healthy donors using the TT suppression assay (see above). Native Fc was used as control. The suppressive response varied by donor, and according to the stimulation parameter measured. V20 at sub-micromolar to low micromolar concentrations suppressed the TT effector response when assessing CD69 (in both donors) or HLA-DR (in one donor) by more than 75% (once the background was subtracted). The order of susceptibility to suppression of the parameters measured as response to stimulation by TT was CD69>HLA-DR>proliferation>CD25. For one of the donors (EV0156), V20 suppressed all four stimulatory parameters tested in this study; strongly with regard to CD69 and HLA-DR, and more weakly with regard to proliferation and CD25. In the second donor (EV0159), V20 showed a strong effect on CD69, a weaker effect on HLA-DR, and no appreciable effect on proliferation or CD25.

Comparison of the Expression of a Recombinant Protein of the Invention with Directly Fused Tregitope Peptides

The goal of this experiment was to compare the expression of a TCP according to the invention with expression of three sequentially cloned tregitopes (direct tregitopes, Dir-Treg-FLAG) N-terminally fused to murine IgG1 signal peptide and C-terminally fused to a FLAG-Tag for detection or potential purification (see below Table and indicated SEQ ID NOs).

| SEQ | | Tregi- | Tregi- | Tregi- | Tregi- | |
|---|---|---|---|---|---|---|
| ID | Con- | tope | tope | tope | tope | FLAG |
| NO | struct | 1 | 2 | 3 | 4 | Seq. |
| 97 & | Dir- | 289Q | 084 | 134 | — | DYKDDDDK |
| 101 | Treg- | | | | | (SEQ ID |
| | 01-FLAG | | | | | NO: 100) |
| 98 & | Dir- | 009a | 088x | 084 | — | DYKDDDDK |
| 102 | Treg- | | | | | (SEQ ID |
| | 02-FLAG | | | | | NO: 100) |
| 99 & | Dir- | 088x | 289Q | 009a | — | DYKDDDDK |
| 103 | Treg- | | | | | (SEQ ID |
| | 03-FLAG | | | | | NO: 100) |
| 92 | Fc_Treg_ | 009a | 084 | 009a | 088x | — |
| | V32 | | | | | |

CAP-T cells were cultured in PEM medium supplemented with 4 mM GlutaMAX (Thermo Fisher Scientific, 35050038) and 5 μg/ml blasticidin (Thermo Fisher Scientific, R21001; complete PEM medium). In order to thaw the cells, the required amount of frozen vials were transferred to a 37° C. water bath. After thawing, each vial was transferred to 10 mL of chilled, complete PEM medium. The cell suspension was centrifuged at 150×g for 5 minutes. During this washing step, the DMSO was removed. The pellet was resuspended in 15 mL warm, complete PEM medium and transferred to a 125 mL shaker flask. The cells were incubated at 37° C. in a humidified incubator with an atmosphere containing 5% CO₂. The flasks were set on a shaking platform, rotating at 185 rpm with an orbit of 50 mm.

Subculturing of the cells was performed every 3 to 4 days. The fresh culture was set to 0.5×10⁶ cells/ml by transferring the required amount of cultured cell suspension to a new flask and adding complete PEM medium. In the case that the transferred cell suspension would exceed 20% of the total volume, the suspension was centrifuged at 150×g for 5 minutes and the pellet was resuspended in fresh complete PEM medium. The volume of cell suspension per shaking flask was 20% of the total flask volume. A minimum of three subcultures were performed after thawing before transfection experiments were performed.

The CAP-T cells were transfected using the 4D-Nucleofector. For each transfection, 10×10⁶ CAP-T cells were centrifuged at 150×g for 5 minutes in 15 ml conical tubes. The cells were resuspended in 95 μL supplemented SE Buffer, taking into account the volume of the pellet and the volume of the plasmid solution. Afterwards, 5 μg of the respective plasmid were added to the cell suspension followed by gentle mixing. The solution was transferred to 100 μL Nucleocuvettes. The used transfection program was ED-100. After the transfection, the cells from one Nucleocuvette were transferred to 125 mL shaker flasks, containing 12.5 mL complete PEM medium. The cells were cultivated for 4 days as described above. At day 4 the cells were harvested by centrifugation at 150×g for 5 minutes.

Supernatants of the protein of the invention were diluted 1:10 and supernatants of Dir-Treg-FLAG were diluted 1:2 with reducing sample buffer. Carboxy-terminal FLAG-BAP Fusion Protein (Sigma-Aldrich, P7457-1MG) was used in a serial dilution (final amount load to the gel: 640 ng, 320 ng, 160 ng, 80 ng, 40 ng, 20 ng) as control. Reducing sample buffer was produced by combining 2.5 parts of NuPAGE LDS Sample Buffer (4×, Thermo Fisher Scientific, NP0007) with 1 part of NuPAGE Sample Reducing Agent (10×, Thermo Fisher Scientific, NP0004). 20 μL of each sample were mixed with 20 μL of reducing sample buffer in a 1.5 mL vial and heated for 10 min at 70° C. using a thermoshaker (Eppendorf). A NuPAGE 4-12% Bis-Tris Protein Gel (Thermo Fisher Scientific) was inserted into the XCell SureLock Mini-Cell Electrophoresis System (Thermo Fisher Scientific) and inner and outer chambers were filled with 1×NuPAGE MES SDS Running buffer (Thermo Fisher Scientific, NP000202). 500 μL of NuPAGE Antioxidant (Thermo Fisher Scientific) was added to the inner chamber. 10 μL of the each prepared sample and 4 μL of Precision Plus Protein All Blue Standard (Bio-Rad, 161-0373) diluted 1/10 in 1×LDS Sample Buffer were loaded onto the gel. The sample separation was achieved by running the gel at a constant voltage of 200 V for 50-60 min.

To investigate the separated proteins by immunofluorescence detection, they were transferred onto an Amersham Hybond Low Fluorescence 0.2 μm polyvinylidene fluoride (PVDF) membrane (GE Healthcare Life Sciences) by using the XCell II Blot module (Thermo Fisher Scientific) for semi-wet protein transfer. The PVDF membrane was directly applied to the SDS gel and the system was filled with NuPAGE Transfer Buffer (20×, Thermo Fisher Scientfic) according to the manufacturer's instructions. Protein blotting was performed for 1 h at 30 V. After protein transfer, the membrane was blocked over night at 4° C. in Odyssey Blocking buffer (Licor) and incubated afterwards simultaneously with 2 μg/mL Monoclonal ANTI-FLAG M2 antibody (Sigma Aldrich, F1804-200UG) and 17 μg/mL AffiniPure Mouse Anti-Human IgG, Fcγ Fragment Specific (Jackson Immuno Research, 209-005-098) diluted in Odyssey Blocking buffer containing 0.05% Tween 20 for 1 h at room temperature. After incubation, the PVDF membrane was washed four times for 5 min in 0.1% PBST. For detection of proteins the membrane was cut into two pieces and the membrane part for FLAG-detection was incubated for 1 h with 0.067 μg/ml of IRDye 800CW Donkey Anti-Mouse (Licor). The other part of the membrane containing the protein of the invention was incubated with IRDye 680RD Donkey Anti-Mouse (Licor). Finally, the PVDF membrane was washed four times for 5 min in 0.1% PBST, two times for 5 min in PBS and rinsed in water. The membrane was visualized using the Licor Odyssey Imager. Band intensities were quantified using the Phoretix 1D software and expression rates between the protein of the invention and Dir-Treg-FLAG were compared.

A concentration-dependent immunofluorescence signal of the latter control protein was observed, demonstrating the quality of the anti-FLAG antibody detection (FIG. 11). Dir-Treg-01-FLAG was hardly expressed. Dir-Treg-03-FLAG was expressed in minimal amounts, while Dir-Treg-02-FLAG demonstrated the best expression of the sequential tregitope peptides. However, the expression of the protein of the invention (construct V32) was 9-times higher compared to Dir-Treg-02-FLAG and 20-times higher compared to Dir-Treg-03-FLAG.

This experiment clearly demonstrated the favorable expression of the TCP according to the invention compared to the expression of three different versions of three sequential tregitope peptides fused to a FLAG-tag.

1. A tregitope carrying polypeptide (TCP) comprising an amino acid sequence having at least 85% sequence identity with amino acids 135 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,
wherein sequence frames A, B, and C are not taken into account for determining the sequence identity.

2. The TCP according to claim 1, wherein the heterologous tregitope does not occur identically in the same position in an amino acid sequence having at least 85% sequence identity to SEQ ID NO: 1.

3. The TCP according to claim 1, comprising at least two heterologous tregitopes, preferably, at least three or four,
wherein, optionally, a first heterologous tregitope is located in one of frames A, B, or C, and wherein at least a second tregitope is located in a different frame of frames A, B, C, or C-terminal to the amino acid sequence having at least 85% sequence identity with amino acids 135 to 330 of SEQ ID NO: 1.

4. The TCP according to claim 1, wherein
(a) if sequence frame A contains no heterologous tregitope, said frame A has at least 85% sequence identity with positions 168 to 203 of SEQ ID NO: 1, and
(b) if sequence frame B contains no heterologous tregitope, said frame B has at least 85% sequence identity with positions 272 to 307 of SEQ ID NO: 1, and
(c) if sequence frame C contains no heterologous tregitope, said frame C has at least 85% sequence identity with positions 212 to 249 of SEQ ID NO: 1.

5. The TCP according to claim 1, wherein the at least one heterologous tregitope substitutes a sequence in any of frames A B or C having the same length as said tregitope or having the length of the tregitope plus or minus one or two amino acids.

6. The TCP according to claim 1, wherein at least one heterologous tregitope is selected from the group consisting of:
SEQ ID NO: 10 (Treg289),
SEQ ID NO: 7 (Treg084),
SEQ ID NO: 2 (Treg009A),
SEQ ID NO: 9 (Treg088x),
SEQ ID NO: 8 (Treg134),
SEQ ID NO: 3 (Treg029B),
SEQ ID NO: 4 (Treg088),
SEQ ID NO: 5 (Treg167),
SEQ ID NO: 6 (Treg289n-native),
SEQ ID NO: 11 (trimmed Treg009A),
SEQ ID NO: 12 (trimmed Treg029B-v1),
SEQ ID NO: 13 (trimmed Treg029B-v2),
SEQ ID NO: 14 (trimmed Treg088),
SEQ ID NO: 15 (trimmed Treg088x-v1),
SEQ ID NO: 16 (trimmed Treg088x-v2),
SEQ ID NO: 17 (trimmed Treg167),
SEQ ID NO: 18 (trimmed Treg289n),
SEQ ID NO: 19 (trimmed Treg289),
SEQ ID NO: 20 (trimmed Treg084), and
SEQ ID NO: 21 (trimmed Treg134);
wherein, preferably, all tregitopes are selected from said group.

7. The TCP according to claim 1, wherein said TCP is selected from the group consisting of
(I) a TCP comprising
(a) a tregitope according to SEQ ID NO: 2 (Treg009A) located in frame A, and
(b) a tregitope according to SEQ ID NO: 2 (Treg009A) located frame B, and
(c) a tregitope according to SEQ ID NO: 7 (Treg084) located in frame C, and
(d) a tregitope according to SEQ ID NO: 9 (Treg088x)C-terminal to the amino acid sequence having at least 85% sequence identity with amino acids 135 to 330 of SEQ ID NO: 1, optionally linked to said sequence via a linker of 3-18 amino acids;
(II) a TCP comprising
(a) a tregitope according to SEQ ID NO: 9 (Treg088x) located in frame B, and
(b) a tregitope according to SEQ ID NO: 2 (Treg009A) located in frame C;
(III) a TCP comprising
(a) a tregitope according to SEQ ID NO: 10 (Treg289) located in frame B, and
(b) a tregitope according to SEQ ID NO: 9 (Treg088x)C-terminal to the amino acid sequence having at least 85% sequence identity with amino acids 135 to 330 of SEQ ID NO: 1, optionally linked to said sequence via a linker of 3-18 amino acids;
(IV) a TCP comprising
(a) a tregitope according to SEQ ID NO: 10 (Treg289) located in frame A, and
(b) a tregitope according to SEQ ID NO: 7 (Treg084) located in frame C, and
(c) a tregitope according to SEQ ID NO: 8 (Treg134)C-terminal to the amino acid sequence having at least 85% sequence identity with amino acids 135 to 330 of SEQ ID NO: 1, optionally linked to said sequence via a linker of 3-18 amino acids;
(V) a TCP comprising
(a) a tregitope according to SEQ ID NO: 10 (Treg289) located in frame A,
(b) a tregitope according to SEQ ID NO: 8 (Treg134) located in frame B, and
(c) a tregitope according to SEQ ID NO: 7 (Treg084) located in frame C;
(VI) a TCP comprising
(a) a tregitope according to SEQ ID NO: 10 (Treg289) located in frame A, and
(b) a tregitope according to SEQ ID NO: 7 (Treg084) located in frame C, and
(c) a tregitope according to SEQ ID NO: 9 (Treg088x)C-terminal to the amino acid sequence having at least 85% sequence identity with amino acids 135 to 330 of SEQ ID NO: 1, optionally linked to said sequence via a linker of 3-18 amino acids; and
(VII) a TCP comprising
(a) a tregitope according to SEQ ID NO: 7 (Treg084) located in frame C, and
(b) a tregitope according to SEQ ID NO: 8 (Treg134)C-terminal to the amino acid sequence having at least 85% sequence identity with amino acids 135 to 330 of SEQ ID NO: 1, optionally linked to said sequence via a linker of 3-18 amino acids.

8. The TCP according to claim 1, wherein the TCP optionally comprises an amino acid sequence selected from the group consisting of SEQ ID NOs: 23 to 44 and 46 to 58 and 111, optionally SEQ ID NO: 54.

9. The TCP according to claim 1, wherein the TCP consists of from 195 to 350 amino acids,
wherein the TCP optionally essentially consists of the amino acid sequence having at least 85% sequence identity with amino acids 135 to 330 of SEQ ID NO: 1, wherein said TCP comprises at least one tregitope heterologous to SEQ ID NO: 1 that is located within at least one of sequence frames A, B, or C, wherein
(a) sequence frame A corresponds to positions 168 to 203 of SEQ ID NO: 1, and
(b) sequence frame B corresponds to positions 272 to 307 of SEQ ID NO: 1, and
(c) sequence frame C corresponds to positions 212 to 249 of SEQ ID NO: 1,
wherein sequence frames A, B, and C are not taken into account for determining the sequence identity,
optionally, with one further tregitope C-terminal to the amino acid sequence having at least 85% sequence identity with amino acids 135 to 330 of SEQ ID NO: 1 that may be linked to said sequence via a linker of 3-18 amino acids.

10. The TCP according to claim 1, wherein the TCP wherein the TCP further comprises a VH domain and CH1 domain of an antibody, preferably, an antigen-binding part of an antibody.

11. The TCP according to claim 1, wherein said TCP forms a multimer comprising at least two, three, four, five, six, or more TCP monomers,
preferably, a dimer comprising at least two TCP monomers according to any one of the preceding claims,
wherein, optionally, said TCP monomers are covalently linked via at least one disulfide bridge.

12. The TCP according to claim 1, wherein the TCP is covalently or non-covalently linked to an agent selected from the group comprising (a) an allergen, (b) an intolerance inducing agent, (c) a target protein of an autoimmune response, e.g., of an autoantibody, (d) a target epitope of an autoimmune response, or (e) a therapeutic agent,
wherein, optionally, the TCP and the agent form a fusion protein.

13. A nucleic acid encoding the TCP according to claim 1,
wherein the nucleic acid optionally is an expression vector suitable for expressing the TCP in an eukaryotic host cell.

14. A host cell comprising the nucleic acid according to claim 13.

15. A method of manufacturing a tregitope carrying polypeptide (TCP), comprising the steps of
(a) cultivating the host cell according to claim 14 under conditions suitable for expression of the TCP
(b) harvesting the cell or medium comprising the TCP expressed in step (a),
(c) isolating said TCP,
(d) optionally, formulating the TCP of step (c) as a pharmaceutically acceptable composition.

16. A transgenic, preferably, non-human animal comprising the nucleic acid according to claim 13.

17. A pharmaceutical composition comprising the TCP according to claim 1, and, optionally, a pharmaceutically acceptable carrier and/or excipient.

18. The pharmaceutical composition according to claim 15 for use in modulating an immune response,
preferably, for suppressing an immune response or inducing tolerance in a subject,
wherein, optionally, said immune response is an immune response to an agent with which the TCP is co-administered, e.g., in covalently linked form.

19. The pharmaceutical composition according to claim 17 for use in the prevention or treatment of an autoimmune related disorder, allergy, viral infection, or transplantation-related immune reaction or disorder in a subject,
preferably, for use in the treatment of an autoimmune disorder.


SEQUENCE LISTING (111 sequences)

SEQ ID NO: 1 - protein, 330 aa, Homo sapiens [sequence omitted]

SEQ ID NO: 2 - protein, 21 aa, Artificial Sequence
  Treg009A
GGLVQPGGSLRLSCAASGFTF

SEQ ID NO: 3 - protein, 15 aa, Artificial Sequence
  Treg029B
MHWVRQAPGKGLEWV

SEQ ID NO: 4 - protein, 21 aa, Artificial Sequence
  Treg088
NTLYLQMNSLRAEDTAVYYCA

SEQ ID NO: 5 - protein, 26 aa, Artificial Sequence
  Treg167
PAVLQSSGLYSLSSVVTVPSSSLGTQ

SEQ ID NO: 6 - protein, 21 aa, Artificial Sequence
  Treg289n - native
EEQYNSTYRVVSVLTVLHQDW

SEQ ID NO: 7 - protein, 15 aa, Artificial Sequence
  Treg084
GTDFTLTISSLQPED

SEQ ID NO: 8 - protein, 24 aa, Artificial Sequence
  Treg134
LNNFYPREAKVQWKVDNALQSGNS

SEQ ID NO: 9 - protein, 21 aa, Artificial Sequence
  Treg088x
KTLYLQMNSLRAEDTAKHYCA

SEQ ID NO: 10 - protein, 21 aa, Artificial Sequence
  Treg289
EEQYQSTYRVVSVLTVLHQDW

SEQ ID NO: 11 - protein, 15 aa, Artificial Sequence
  trimmed Treg009A
VQPGGSLRLSCAASG

SEQ ID NO: 12 - protein, 10 aa, Artificial Sequence
  trimmed Treg029B - v1
WVRQAPGKGL

SEQ ID NO: 13 - protein, 9 aa, Artificial Sequence
  trimmed Treg029B - v2
VRQAPGKGL

SEQ ID NO: 14 - protein, 15 aa, Artificial Sequence
  trimmed Treg088
YLQMNSLRAEDTAVY

SEQ ID NO: 15 - protein, 18 aa, Artificial Sequence
  trimmed Treg088x - v1
KTLYLQMNSLRAEDTAKH

SEQ ID NO: 16 - protein, 15 aa, Artificial Sequence
  trimmed Treg088x - v2
YLQMNSLRAEDTAKH

SEQ ID NO: 17 - protein, 20 aa, Artificial Sequence
  trimmed Treg167
LQSSGLYSLSSVVTVPSSSL

SEQ ID NO: 18 - protein, 15 aa, Artificial Sequence
  trimmed Treg289n
YNSTYRVVSVLTVLH

SEQ ID NO: 19 - protein, 15 aa, Artificial Sequence
  trimmed Treg289
YQSTYRVVSVLTVLH

SEQ ID NO: 20 - protein, 9 aa, Artificial Sequence
  trimmed Treg084
FTLTISSLQ

SEQ ID NO: 21 - protein, 18 aa, Artificial Sequence
  trimmed Treg134
FYPREAKVQWKVDNALQS

SEQ ID NO: 22 - protein, 20 aa, Artificial Sequence
  Fc signal peptide
METDTLLLWVLLLWVPGSTG

SEQ ID NO: 23 - protein, 251 aa, Artificial Sequence
  FcTreg-V1
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGTDFTLTISSLQPEDDELTKNQVSLTCLVKGFYPSDIAVEWESNGQPENNYKTTPPVLDSDGSFFLYSKLTVDKSRWQQGNVFSCSVMHEALHNHYTQKSLSLSPGKLNNFYPREAKVQWKVDNALQSGNS

SEQ ID NO: 24 - protein, 248 aa, Artificial Sequence
  FcTreg-V2
DKTHTCPPCPAPELLGGPSVFLFPPKPKDTLMISRTPEVTCVVVDVSHEDPEVKFNWYVDGVEVHNAKTKPAVLQSSGLYSLSSVVTVPSSSLGTQKEYKCKVSNKALPAPIEKTISKAMHWVRQAPGKGLEWVRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGLVQPGGSLRLSCAASGFTF

SEQ ID NO: 25 - protein, 227 aa, Artificial Sequence
  FcTreg-V3
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGTDFTLTISSLQPEDDELTKNQVSLTCLVKGFYPSDIAVEWESNGQPENNYKTLNNFYPREAKVQWKVDNALQSGNSQQGNVFSCSVMHEALHNHYTQKSLSLSPGK

SEQ ID NO: 26 - protein, 242 aa, Artificial Sequence
  FcTreg-V4
DKTHTCPPCPAPELLGGPSVFLFPPKPKDTLMISRTPEVTCVVVDVSHEDPEVKFNWYVDGVEVHNAKTKPREEQYGGLVQPGGSLRLSCAASGFTFEYKCKVSNKALPAPIEKTISKAKGTDFTLTISSLQPEDDELTKNQVSLTCLVKGFYPSDIAVEWESNGQPENNYKTTPPVLDSDGSFFLYSKLTVDKSRWQQGNVFSCSVMHEALHNHYTQKSLSLSPGKMHWVRQAPGKGLEWV

SEQ ID NO: 27 - protein, 251 aa, Artificial Sequence
  FcTreg-V5
DKTHTCPPCPAPELLGGPSVFLFPPKPKDTLMISRTPEVTCVVVDVSHEDPEVKFNWYVDGVEVHNAKTKPREEMHWVRQAPGKGLEWVHQDWLNGKEYKCKVSNKALPAPIEKTISKAPAVLQSSGLYSLSSVVTVPSSSLGTQTCLVKGFYPSDIAVEWESNGQPENNYKTTPPVLDSDGSFFLYSKLTVDKSRWQQGNVFSCSVMHEALHNHYTQKSLSLSPGKLNNFYPREAKVQWKVDNALQSGNS

SEQ ID NO: 28 - protein, 227 aa, Artificial Sequence
  FcTreg-V6
DKTHTCPPCPAPELLGGPSVFLFPPKPKDTLMISRTPEVTCVVVDVSHEDPEVKFNWYVDGVEVHNAKTKPREEQYNSTYRVGTDFTLTISSLQPEDEYKCKVSNKALPAPIEKTISKAGGLVQPGGSLRLSCAASGFTFNQVSLTCLVKGFYPSDIAVEWESNGQPENNYKTTPAVLQSSGLYSLSSVVTVPSSSLGTQNVFSCSVMHEALHNHYTQKSLSLSPGK

SEQ ID NO: 29 - protein, 227 aa, Artificial Sequence
  FcTreg-V7
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGEEQYQSTYRVVSVLTVLHQDWVSLTCLVKGFYPSDIAVEWESNGQPENNYKTTPPEEQYQSTYRVVSVLTVLHQDWQQGNVFSCSVMHEALHNHYTQKSLSLSPGK

SEQ ID NO: 30 - protein, 253 aa, Artificial Sequence
  FcTreg-V8
DKTHTCPPCPAPELLGGPSVFLFPPKPKDTLMISRTPEVTCVVVDVSHEDPEVKFNWYVDGVEVHNAKTKPAVLQSSGLYSLSSVVTVPSSSLGTQKEYKCKVSNKALPAPIEKTISKAPAVLQSSGLYSLSSVVTVPSSSLGTQTCLVKGFYPSDIAVEWESNGQPENNYKTTPPVLDSDGSFFLYSKLTVDKSRWQQGNVFSCSVMHEALHNHYTQKSLSLSPGKPAVLQSSGLYSLSSVVTVPSSSLGTQ

SEQ ID NO: 31 - protein, 227 aa, Artificial Sequence
  FcTreg-V9
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGTDFTLTISSLQPEDDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 227 aa, Artificial Sequence
  FcTreg-V10
DKTHTCPPCPAPELLGGPSVFLFPPKPKDTLMISRTPEVTCVVVDVSHEDPEVKFNWYVDGVEVHNAKTKPAVLQSSGLYSLSSVVTVPSSSLGTQKEYKCKVSNKALPAPIEKTISKLNNFYPREAKVQWKVDNALQSGNSVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 227 aa, Artificial Sequence
  FcTreg-V11
DKTHTCPPCPAPELLGGPSVFLFPPKPKDTLMISRTPEVTCVVVDVSHEDPEVKFNWYVDGVEVHNAKTKPAVLQSSGLYSLSSVVTVPSSSLGTQKEYKCKVSNKALPAPIEKTISKAKGEEQYQSTYRVVSVLTVLHQDWVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 251 aa, Artificial Sequence
  FcTreg-V12
DKTHTCPPCPAPELLGGPSVFLFPPKPKDTLMISRTPEVTCVVVDVSHEDPEVKFNWYVDGVEVHNAKTKPREEQYGGLVQPGGSLRLSCAASGFTFEYKCKVSNKALPAPIEKTISKAKGEEQYQSTYRVVSVLTVLHQDWVSLTCLVKGFYPSDIAVEWESNGQPENNYKTTPPVLDSDGSFFLYSKLTVDKSRWQQGNVFSCSVMHEALHNHYTQKSLSLSPGKLNNFYPREAKVQWKVDNALQSGNS

SEQ ID NO: 35 - protein, 245 aa, Artificial Sequence
  FcTreg-V13
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGTDFTLTISSLQPEDDELTKNQVSLTCLVKGFYPSDIAVEWESNGQPENNYKTTPPVLDSDGSFFLYSKLTVDKSRWQQGNVFSCSVMHEALHNHYTQKSLSLSPGKKTLYLQMNSLRAEDTAKH

SEQ ID NO: 36 - protein, 251 aa, Artificial Sequence
  FcTreg-V14
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGTDFTLTISSLQPEDDELTKNQVSLTCLVKGFYPSDIAVEWESNGQPENNYKTTPPVLDSDGSFFLYSKLTVDKSRWQQGNVFSCSVMHEALHNHYTQKSLSLSPGKLNNFYPREAKVQWKVDNALQSGNS

SEQ ID NO: 37 - protein, 245 aa, Artificial Sequence
  FcTreg-V15
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGEEQYQSTYRVVSVLTVLHQDWVSLTCLVKGFYPSDIAVEWESNGQPENNYKTTPPVLDSDGSFFLYSKLTVDKSRWQQGNVFSCSVMHEALHNHYTQKSLSLSPGKKTLYLQMNSLRAEDTAKH

SEQ ID NO: 38 - protein, 248 aa, Artificial Sequence
  FcTreg-V16
DKTHTCPPCPAPELLGGPSVFLFPPKPKDTLMISRTPEVTCVVVDVSHEDPEVKFNWYVDGVEVHNAKTKPREEQYNSTYRVGTDFTLTISSLQPEDEYKCKVSNKALPAPIEKTISKAKGEEQYQSTYRVVSVLTVLHQDWVSLTCLVKGFYPSDIAVEWESNGQPENNYKTTPPEEQYQSTYRVVSVLTVLHQDWQQGNVFSCSVMHEALHNHYTQKSLSLSPGKEEQYQSTYRVVSVLTVLHQDW

SEQ ID NO: 39 - protein, 242 aa, Artificial Sequence
  FcTreg-V17
DKTHTCPPCPAPELLGGPSVFLFPPKPKDTLMISRTPEVTCVVVDVSHEDPEVKFNWYVDGVEVHNAKTKPREEQYGGLVQPGGSLRLSCAASGFTFEYKCKVSNKALPAPIEKTISKAGGLVQPGGSLRLSCAASGFTFNQVSLTCLVKGFYPSDIAVEWESNGQPENNYKTTPPVLDSDGSFFLYSKLTVDKSRWQQGNVFSCSVMHEALHNHYTQKSLSLSPGKGTDFTLTISSLQPED

SEQ ID NO: 40 - protein, 248 aa, Artificial Sequence
  FcTreg-V18
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGEEQYQSTYRVVSVLTVLHQDWVSLTCLVKGFYPSDIAVEWESNGQPENNYKTTGGLVQPGGSLRLSCAASGFTFRWQQGNVFSCSVMHEALHNHYTQKSLSLSPGKGGLVQPGGSLRLSCAASGFTF

SEQ ID NO: 41 - protein, 262 aa, Artificial Sequence
  FcTreg-V19
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGTDFTLTISSLQPEDDELTKNQVSLTCLVKGFYPSDIAVEWESNGQPENNYKTTPPVLDSGTDFTLTISSLQPEDRWQQGNVFSCSVMHEALHNHYTQKSLSLSPGKGTDFTLTISSLQPED

SEQ ID NO: 42 - protein, 227 aa, Artificial Sequence
  FcTreg-V20
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAGGLVQPGGSLRLSCAASGFTFNQVSLTCLVKGFYPSDIAVEWESNGQPENNYKTTPPVLDSDGSKTLYLQMNSLRAEDTAKHVFSCSVMHEALHNHYTQKSLSLSPGK

SEQ ID NO: 43 - protein, 249 aa, Artificial Sequence
  FcTreg-V21
DKTHTCPPCPAPELLGGPSVFLFPPKPKDTLMISRTPEVTCVVVDVSHEDPEVKFNWYVDGVEVHNAKTKPREEQYGGLVQPGGSLRLSCAASGFTFEYKCKVSNKALPAPIEKTISKAKGQPREPQVYTLPPSRDELTKNQVSLTCLVKGFYPSDIAVEWESNGQPENNYKTTPPVLDSDGSKTLYLQMNSLRAEDTAKHVFSCSVMHEALHNHYTQKSLSLSPGKGGLVQPGGSLRLSCAASGFTFK

SEQ ID NO: 44 - protein, 248 aa, Artificial Sequence
  FcTreg-V22
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTLYLQMNSLRAEDTAKHPPSRDELTKNQVSLTCLVKGFYPSDIAVEWESNGQPENNYKTTPPEEQYQSTYRVVSVLTVLHQDWQQGNVFSCSVMHEALHNHYTQKSLSLSPGKGGLVQPGGSLRLSCAASGFTF

SEQ ID NO: 45 - protein, 227 aa, Artificial Sequence
  FcTreg-V23
DKTHTCPPCPAPELLGGPSVFLFPPKPKDTLMISRTPEVTCVVVDVSHEDPEVKFNWYVDGVEVHNAKTKPREEQYNSTYRVGTDFTLTISSLQPEDEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 227 aa, Artificial Sequence
  FcTreg-V24
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGTDFTLTISSLQPEDDELTKNQVSLTCLVKGFYPSDIAVEWESNGQPENNYKTTPPEEQYQSTYRVVSVLTVLHQDWQQGNVFSCSVMHEALHNHYTQKSLSLSPGK

SEQ ID NO: 47 - protein, 227 aa, Artificial Sequence
  FcTreg-V25
DKTHTCPPCPAPELLGGPSVFLFPPKPKDTLMISRTPEVTCVVVDVSHEDPEVKFNWYVDGVEVHNAKTKPREEQYGGLVQPGGSLRLSCAASGFTFEYKCKVSNKALPAPIEKTISKAKGEEQYQSTYRVVSVLTVLHQDWVSLTCLVKGFYPSDIAVEWESNGQPENNYKTTPPVLDSGTDFTLTISSLQPEDRWQQGNVFSCSVMHEALHNHYTQKSLSLSPGK

SEQ ID NO: 48 - protein, 248 aa, Artificial Sequence
  FcTreg-V26
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSGTDFTLTISSLQPEDRWQQGNVFSCSVMHEALHNHYTQKSLSLSPGKEEQYQSTYRVVSVLTVLHQDW

SEQ ID NO: 49 - protein, 248 aa, Artificial Sequence
  FcTreg-V27
DKTHTCPPCPAPELLGGPSVFLFPPKPKDTLMISRTPEVTCVVVDVSHEDPEVKFNWYVDGVEVHNAKTKPREEQYNSTYRVGTDFTLTISSLQPEDEYKCKVSNKALPAPIEKTISKAGGLVQPGGSLRLSCAASGFTFNQVSLTCLVKGFYPSDIAVEWESNGQPENNYKTTPPVLDSGTDFTLTISSLQPEDRWQQGNVFSCSVMHEALHNHYTQKSLSLSPGKGGLVQPGGSLRLSCAASGFTF

SEQ ID NO: 50 - protein, 248 aa, Artificial Sequence
  FcTreg-V28
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTLYLQMNSLRAEDTAKHPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEEQYQSTYRVVSVLTVLHQDW

SEQ ID NO: 51 - protein, 242 aa, Artificial Sequence
  FcTreg-V29
DKTHTCPPCPAPELLGGPSVFLFPPKPKDTLMISRTPEVTCVVVDVSHEDPEVKFNWYVDGVEVHNAKTKTLYLQMNSLRAEDTAKHVLHQDWLNGKEYKCKVSNKALPAPIEKTISKAKGEEQYQSTYRVVSVLTVLHQDWVSLTCLVKGFYPSDIAVEWESNGQPENNYKTTPPVLDSDGSKTLYLQMNSLRAEDTAKHVFSCSVMHEALHNHYTQKSLSLSPGKGTDFTLTISSLQPED

SEQ ID NO: 52 - protein, 245 aa, Artificial Sequence
  FcTreg-V30
DKTHTCPPCPAPELLGGPSVFLFPPKPKDTLMISRTPEVTCVVVDVSHEDPEVKFNWYVDGVEVHNAKTKTLYLQMNSLRAEDTAKHVLHQDWLNGKEYKCKVSNKALPAPIEKTLYLQMNSLRAEDTAKHPPSRDELTKNQVSLTCLVKGFYPSDIAVEWESNGQPENNYKTTPPVLDSGTDFTLTISSLQPEDRWQQGNVFSCSVMHEALHNHYTQKSLSLSPGKKTLYLQMNSLRAEDTAKH

SEQ ID NO: 53 - protein, 242 aa, Artificial Sequence
  FcTreg-V31
DKTHTCPPCPAPELLGGPSVFLFPPKPKDTLMISRTPEVTCVVVDVSHEDPEVKFNWYVDGVEVHNAKTKPREEQYNSTYRVGTDFTLTISSLQPEDEYKCKVSNKALPAPIEKTLYLQMNSLRAEDTAKHPPSRDELTKNQVSLTCLVKGFYPSDIAVEWESNGQPENNYKTTGGLVQPGGSLRLSCAASGFTFRWQQGNVFSCSVMHEALHNHYTQKSLSLSPGKGTDFTLTISSLQPED

SEQ ID NO: 54 - protein, 246 aa, Artificial Sequence
  FcTreg-V32
DKTHTCPPCPAPELLGGPSVFLFPPKPKDTLMISRTPEVTCVVVDVSHEDPEVKFNWYVDGVEVHNAKTKPREEQYGGLVQPGGSLRLSCAASGFTFEYKCKVSNKALPAPIEKTISKAKGTDFTLTISSLQPEDRDELTKNQVSLTCLVKGFYPSDIAVEWESNGQPENNYKTTGGLVQPGGSLRLSCAASGFTFRWQQGNVFSCSVMHEALHNHYTQKSLSLSPGKKTLYLQMNSLRAEDTAKH

SEQ ID NO: 55 - protein, 245 aa, Artificial Sequence
  FcTreg-V33
DKTHTCPPCPAPELLGGPSVFLFPPKPKDTLMISRTPEVTCVVVDVSHEDPEVKFNWYVDGVEVHNAKTKPREEQYNSTYRVGTDFTLTISSLQPEDEYKCKVSNKALPAPIEKTISKAKGTDFTLTISSLQPEDDELTKNQVSLTCLVKGFYPSDIAVEWESNGQPENNYKTTPPVLDSDGSKTLYLQMNSLRAEDTAKHVFSCSVMHEALHNHYTQKSLSLSPGKKTLYLQMNSLRAEDTAKH

SEQ ID NO: 56 - protein, 245 aa, Artificial Sequence
  FcTreg-V34
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPEEQYQSTYRVVSVLTVLHQDWQQGNVFSCSVMHEALHNHYTQKSLSLSPGKKTLYLQMNSLRAEDTAKH

SEQ ID NO: 57 - protein, 248 aa, Artificial Sequence
  FcTreg-V35
DKTHTCPPCPAPELLGGPSVFLFPPKPKDTLMISRTPEVTCVVVDVSHEDPEVKFNWYVDGVEVHNAKTKTLYLQMNSLRAEDTAKHVLHQDWLNGKEYKCKVSNKALPAPIEKTISKAGGLVQPGGSLRLSCAASGFTFNQVSLTCLVKGFYPSDIAVEWESNGQPENNYKTTGGLVQPGGSLRLSCAASGFTFRWQQGNVFSCSVMHEALHNHYTQKSLSLSPGKEEQYQSTYRVVSVLTVLHQDW

SEQ ID NO: 58 - protein, 245 aa, Artificial Sequence
  FcTreg-V36
DKTHTCPPCPAPELLGGPSVFLFPPKPKDTLMISRTPEVTCVVVDVSHEDPEVKFNWYVDGVEVHNAKTKTLYLQMNSLRAEDTAKHVLHQDWLNGKEYKCKVSNKALPAPIEGTDFTLTISSLQPEDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGLVQPGGSLRLSCAASGFTF

SEQ ID NO: 59 - DNA, 60 bp, Artificial Sequence
  Fc signal peptide
atggaaaccgacacactgctgctgtgggtgctgcttttgtgggtgccaggcagcaccggc

SEQ ID NO: 60 - protein, 227 aa, Homo sapiens
  Residues 104-330 of UNIPROT sequence P01857
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 753 bp, Artificial Sequence
  FcTregV1
gacaagacccacacctgtcctccctgccctgcccctgagctgctgggcggacccagcgtgttcctgttcccacccaagcccaaggacaccctgatgatcagccggacccctgaagtgacctgcgtggtggtggacgtgtcccacgaggatcccgaagtgaagttcaattggtacgtggacggcgtggaagtgcacaacgccaagaccaagcccagagaggaacagtaccagagcacctaccgggtggtgtccgtgctgaccgtgctgcaccaggactggctgaacggcaaagagtacaagtgcaaggtgtccaacaaggccctgcctgccccaatcgagaaaaccatcagcaaggccaagggcaccgacttcaccctgaccatcagcagcctgcagcccgaggacgacgagctgaccaagaaccaggtgtccctgacctgcctggtcaagggcttctaccccagcgatatcgccgtggaatgggagagcaacggccagcccgagaacaactacaagaccacccctcccgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagctgaacaacttctacccacgcgaggccaaggtgcagtggaaggtggacaatgccctgcagagcggcaacagc

SEQ ID NO: 62 - DNA, 744 bp, Artificial Sequence
  FcTregV2
gacaagacccacacctgtcctccctgccctgctcctgaactgctgggcggacccagcgtgttcctgttcccacccaagcccaaggacaccctgatgatcagccggacccctgaagtgacctgcgtggtggtggacgtgtcccacgaggatcccgaagtgaagttcaattggtacgtggacggcgtggaagtgcacaacgccaagaccaagcccgccgtgctgcagagcagcggcctgtacagcctgagcagcgtggtcaccgtgcccagcagcagcctgggcacccagaaagagtacaagtgcaaggtgtccaacaaggccctgcctgccccaatcgagaaaaccatcagcaaggccatgcactgggtccgacaggcccctggcaagggactggaatgggtccgagatgagctgaccaagaaccaggtgtccctgacctgcctggtcaagggattctaccccagcgatatcgccgtggaatgggagagcaacggccagcccgagaacaactacaagaccacccctcccgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagcccaggcaagggtggactggtgcagcctggcggcagcctgagactgtcttgcgccgccagcggcttcaccttc

SEQ ID NO: 63 - DNA, 681 bp, Artificial Sequence
  FcTregV3
gacaagacccacacctgtcctccctgccctgcccctgagctgctgggcggacccagcgtgttcctgttcccacccaagcccaaggacaccctgatgatcagccggacccctgaagtgacctgcgtggtggtggacgtgtcccacgaggatcccgaagtgaagttcaattggtacgtggacggcgtggaagtgcacaacgccaagaccaagcccagagaggaacagtaccagagcacctaccgggtggtgtccgtgctgaccgtgctgcaccaggactggctgaacggcaaagagtacaagtgcaaggtgtccaacaaggccctgcctgccccaatcgagaaaaccatcagcaaggccaagggcaccgacttcaccctgaccatcagcagcctgcagcccgaggacgacgagctgaccaagaaccaggtgtccctgacctgcctggtcaagggattctaccccagcgatatcgccgtggaatgggagagcaacggccagcccgagaacaactacaagaccctgaacaacttctacccacgcgaggccaaggtgcagtggaaggtggacaatgccctgcagagcggcaacagccagcagggcaacgtgttcagctgcagcgtgatgcacgaggccctgcacaaccactacacccagaagtccctgagcctgagccccggcaag

SEQ ID NO: 64 - DNA, 726 bp, Artificial Sequence
  FcTregV4
gacaagacccacacctgtcctccatgccctgcacctgagctgctgggcggacccagcgtgttcctgttcccacccaagcccaaggacaccctgatgatcagccggacccctgaagtgacctgcgtggtggtggacgtgtcccacgaggatcccgaagtgaagttcaattggtacgtggacggcgtggaagtgcacaacgccaagaccaagcccagagaggaacagtacggcggcctggtgcagcctggcggcagcctgagactgtcttgcgccgccagcggcttcaccttcgagtacaagtgcaaggtgtccaacaaggccctgcctgccccaatcgagaaaaccatcagcaaggccaagggcaccgacttcaccctgaccatcagcagcctgcagcccgaggacgacgagctgaccaagaaccaggtgtccctgacctgcctggtcaagggcttctaccccagcgatatcgccgtggaatgggagagcaacggccagcccgagaacaactacaagaccacccctcccgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagatgcactgggtccgacaggcccctggcaagggcctggaatgggtg

SEQ ID NO: 65 - DNA, 753 bp, Artificial Sequence
  FcTregV5
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagatgcactgggtgcgccaggcccccggcaagggcctggagtgggtgcaccaggactggctgaatggcaaggagtacaagtgcaaggtctccaacaaagccctcccagcccccatcgagaaaaccatctccaaagcccccgccgtgctgcagagcagcggcctgtacagcctgagcagcgtggtgaccgtgcccagcagcagcctgggcacccagacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaactgaacaacttctacccccgcgaggccaaggtgcagtggaaggtggacaacgccctgcagagcggcaacagc

SEQ ID NO: 66 - DNA, 681 bp, Artificial Sequence
  FcTregV6
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtgggcaccgacttcaccctgaccatcagcagcctgcagcccgaggacgagtacaagtgcaaggtctccaacaaagccctcccagcccccatcgagaaaaccatctccaaagccggcggcctggtgcagcccggcggcagcctgcgcctgagctgcgccgccagcggcttcaccttcaaccaggtcagcctgacctgcctggtcaaaggcttctatcccagcgacatcgccgtggagtgggagagcaatgggcagccggagaacaactacaagaccacgcccgccgtgctgcagagcagcggcctgtacagcctgagcagcgtggtgaccgtgcccagcagcagcctgggcacccagaacgtcttctcatgctccgtgatgcatgaggctctgcacaaccactacacgcagaagagcctctccctgtctccgggtaaa

SEQ ID NO: 67 - DNA, 681 bp, Artificial Sequence
  FcTregV7
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacctaccgcgtggtgagcgtgctgaccgtgctgcaccaggactggctgaatggcaaggagtacaagtgcaaggtctccaacaaagccctcccagcccccatcgagaaaaccatctccaaagccaaaggggaggagcagtaccagagcacctaccgcgtggtgagcgtgctgaccgtgctgcaccaggactgggtcagcctgacctgcctggtcaaaggcttctatcccagcgacatcgccgtggagtgggagagcaatgggcagccggagaacaactacaagaccacgcctcccgaggagcagtaccagagcacctaccgcgtggtgagcgtgctgaccgtgctgcaccaggactggcagcaggggaacgtcttctcatgctccgtgatgcatgaggctctgcacaaccactacacgcagaagagcctctccctgtctccgggtaaa

SEQ ID NO: 68 - DNA, 759 bp, Artificial Sequence
  FcTregV8
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagcccgccgtgctgcagagcagcggcctgtacagcctgagcagcgtggtgaccgtgcccagcagcagcctgggcacccagaaggagtacaagtgcaaggtctccaacaaagccctcccagcccccatcgagaaaaccatctccaaagcccccgccgtgctgcagagcagcggcctgtacagcctgagcagcgtggtgaccgtgcccagcagcagcctgggcacccagacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacccgccgtgctgcagagcagcggcctgtacagcctgagcagcgtggtgaccgtgcccagcagcagcctgggcacccag

SEQ ID NO: 69 - DNA, 681 bp, Artificial Sequence
  FcTregV9
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacctaccgcgtggtgagcgtgctgaccgtgctgcaccaggactggctgaatggcaaggagtacaagtgcaaggtctccaacaaagccctcccagcccccatcgagaaaaccatctccaaagccaaaggcaccgacttcaccctgaccatcagcagcctgcagcccgaggacgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 70 - DNA, 681 bp, Artificial Sequence
  FcTregV10
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagcccgccgtgctgcagagcagcggcctgtacagcctgagcagcgtggtgaccgtgcccagcagcagcctgggcacccagaaggagtacaagtgcaaggtctccaacaaagccctcccagcccccatcgagaaaaccatctccaaactgaacaacttctacccccgcgaggccaaggtgcagtggaaggtggacaacgccctgcagagcggcaacagcgtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 71 - DNA, 681 bp, Artificial Sequence
  FcTregV11
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagcccgccgtgctgcagagcagcggcctgtacagcctgagcagcgtggtgaccgtgcccagcagcagcctgggcacccagaaggagtacaagtgcaaggtctccaacaaagccctcccagcccccatcgagaaaaccatctccaaagccaaaggggaggagcagtaccagagcacctaccgcgtggtgagcgtgctgaccgtgctgcaccaggactgggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 72 - DNA, 753 bp, Artificial Sequence
  FcTregV12
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggcggcctggtgcagcccggcggcagcctgcgcctgagctgcgccgccagcggcttcaccttcgagtacaagtgcaaggtctccaacaaagccctcccagcccccatcgagaaaaccatctccaaagccaaaggggaggagcagtaccagagcacctaccgcgtggtgagcgtgctgaccgtgctgcaccaggactgggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaactgaacaacttctacccccgcgaggccaaggtgcagtggaaggtggacaacgccctgcagagcggcaacagc

SEQ ID NO: 73 - DNA, 735 bp, Artificial Sequence
  FcTregV13
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacctaccgcgtggtgagcgtgctgaccgtgctgcaccaggactggctgaatggcaaggagtacaagtgcaaggtctccaacaaagccctcccagcccccatcgagaaaaccatctccaaagccaaaggcaccgacttcaccctgaccatcagcagcctgcagcccgaggacgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaagaccctgtacctgcagatgaacagcctgagggccgaggacaccgccaagcac

SEQ ID NO: 74 - DNA, 753 bp, Artificial Sequence
  FcTregV14
gacaaaactcacacatgcccaccgtgcccagcacctgaactcctgggaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacctaccgcgtggtgagcgtgctgaccgtgctgcaccaggactggctgaatggcaaggagtacaagtgcaaggtctccaacaaagccctcccagcccccatcgagaaaaccatctccaaagccaaaggcaccgacttcaccctgaccatcagcagcctgcagcccgaggacgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaactgaacaacttctacccccgcgaggccaaggtgcagtggaaggtggacaacgccctgcagagcggcaacagc

SEQ ID NO: 75 - DNA, 735 bp, Artificial Sequence
  FcTregV15
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtaccagagcacctacagagtggtgtccgtgctgacagtgctgcaccaggactggctgaacggcaaagagtacaagtgcaaggtgtccaacaaggccctgcctgctcctatcgagaaaaccatcagcaaggccaagggcgaagaacagtatcagtccacctaccgggtcgtcagcgttctgaccgttctgcatcaggattgggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgaccgtggacaagagcagatggcagcagggcaatgtgttcagctgctctgtgatgcacgaggccctgcacaaccactacacccagaagtctctgtctctgagccccggcaagaaaaccctgtacctgcagatgaactccctgagagccgaggacaccgccaagcac

SEQ ID NO: 76 - DNA, 744 bp, Artificial Sequence
  FcTregV16
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtgggcaccgacttcaccctgaccatcagttctctgcagcccgaggacgagtacaagtgcaaggtgtccaacaaggccctgcctgctcctatcgagaaaaccatcagcaaggccaagggcgaagaacagtaccagagcacataccgcgtggtgtctgtgctgacagtgctgcatcaggattgggtgtccctgacctgcctggtcaagggcttctacccttccgatatcgccgtggaatgggagagcaatggccagcctgagaacaactacaagaccacacctcctgaggaacaataccagtccacatatagagtggtgtccgtcctcaccgtcctgcaccaagattggcagcagggcaatgtgttcagctgctctgtgatgcacgaggccctgcacaaccactacacccagaagtctctgtctctgagccccggcaaagaagaacaatatcaatccacctaccgggtcgtcagcgtgctcaccgtgcttcatcaagactgg

SEQ ID NO: 77 - DNA, 726 bp, Artificial Sequence
  FcTregV17
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacggcggactggttcaacctggcggctctctgagactgtcttgtgccgccagcggcttcaccttcgagtacaagtgcaaggtgtccaacaaggccctgcctgctcctatcgagaaaaccatctctaaggctggcggactcgtgcagccaggtggaagtcttagactgagctgtgccgcctctggctttaccttcaatcaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgaccgtggacaagagcagatggcagcagggcaatgtgttcagctgctctgtgatgcacgaggccctgcacaaccactacacccagaagtctctgtctctgagccctggcaagggcaccgatttcaccctgaccatttctagcctgcagcctgaggac

SEQ ID NO: 78 - DNA, 744 bp, Artificial Sequence
  FcTregV18
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggcgaagaacagtaccagagcacataccgggtcgtcagcgttctgacagtgctccatcaagactgggtgtccctgacctgcctggtcaagggcttctacccttccgatatcgccgtggaatgggagagcaatggccagcctgagaacaactacaagaccaccggcggactggttcagcctggtggatctctgagactgtcttgtgccgccagcggcttcacctttagatggcagcagggcaacgtgttcagctgctctgtgatgcacgaggccctgcacaaccactacacccagaagtctctgtctctgagccctggaaaaggcggacttgtgcaaccaggtggaagcctgagactgagctgtgccgcttccggctttaccttc

SEQ ID NO: 79 - DNA, 786 bp, Artificial Sequence
  FcTregV19
atggaaaccgacacactgctgctgtgggtgctgcttttgtgggtgccaggcagcaccggcgataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggcaccgacttcaccctgaccattagtagcctgcagcctgaggacgacgagctgaccaaaaatcaggtgtccctgacctgcctggtcaagggcttctacccttccgatatcgccgtggaatgggagagcaatggccagcctgagaacaactacaagaccacacctcctgtgctggacagcggcacagatttcacactgacaatcagctcactgcagcccgaagatagatggcagcagggcaatgtgttcagctgctctgtgatgcacgaggccctgcacaaccactacacccagaagtctctgtctctgagccccggcaagggaacagactttacccttacaatctcatccctccagccagaggac

SEQ ID NO: 80 - DNA, 681 bp, Artificial Sequence
  FcTregV20
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatctctaaggctggcggcctggttcagcctggtggatctctgagactgtcttgtgccgccagcggcttcaccttcaatcaggtgtccctgacctgcctggtcaagggcttctacccttccgatatcgccgtggaatgggagagcaatggccagcctgagaacaactacaagaccacacctcctgtgctggacagcgacggcagcaagacactgtacctgcagatgaactccctgagagccgaggataccgccaagcacgtgttcagctgttctgtgatgcacgaggccctgcacaaccactacacccagaagtctctgtctctgagccccggcaag

SEQ ID NO: 81 - DNA, 744 bp, Artificial Sequence
  FcTregV21
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacggcggactggttcaacctggcggctctctgagactgtcttgtgccgccagcggcttcaccttcgagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagaccacacctcctgtgctggacagcgacggcagcaagacactgtacctgcagatgaactccctgagagccgaggataccgccaagcacgtgttcagctgttctgtgatgcacgaggccctgcacaaccactacacccagaagtctctgtctctgagccctggaaaaggtggacttgtgcagccaggtggaagcctgagactgagctgtgccgcttccggctttaccttc

SEQ ID NO: 82 - DNA, 744 bp, Artificial Sequence
  FcTregV22
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtaccagagcacctacagagtggtgtccgtgctgacagtgctgcaccaggactggctgaacggcaaagagtacaagtgcaaggtgtccaacaaggccctgcctgctcctatcgagaaaaccctgtacctgcagatgaacagcctgagagccgaggacacagctaagcaccctccaagcagagatgagctgaccaagaaccaggtgtccctgacctgcctggtcaagggcttctacccttccgatatcgccgtggaatgggagagcaatggccagccagagaacaactacaagaccacacctcctgaagaacagtatcagtccacctaccgggtcgtcagcgttctgaccgttctgcatcaggattggcagcagggcaacgtgttcagctgttctgtgatgcacgaggccctgcacaaccactacacccagaagtctctgtctctgagccctggcaaaggcggacttgttcaacctggcggctctctgagactgtcttgtgccgccagcggcttcaccttc

SEQ ID NO: 83 - DNA, 681 bp, Artificial Sequence
  FcTregV23
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtgggcaccgacttcaccctgaccatcagttctctgcagcccgaggacgagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgaccgtggacaagagcagatggcagcagggcaatgtgttcagctgctctgtgatgcacgaggccctgcacaaccactacacccagaagtctctgtctctgagccccggcaag

SEQ ID NO: 84 - DNA, 681 bp, Artificial Sequence
  FcTregV24
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtaccagagcacctacagagtggtgtccgtgctgacagtgctgcaccaggactggctgaacggcaaagagtacaagtgcaaggtgtccaacaaggccctgcctgctcctatcgagaaaaccatcagcaaggccaagggcaccgacttcaccctgaccattagtagcctgcagcctgaggacgacgagctgaccaaaaatcaggtgtccctgacctgcctggtcaagggcttctacccttccgatatcgccgtggaatgggagagcaatggccagcctgagaacaactacaagaccacacctcctgaagaacagtatcagtccacctaccgggtcgtcagcgttctgaccgttctgcatcaggattggcagcagggcaacgtgttcagctgttctgtgatgcacgaggccctgcacaaccactacacccagaagtctctgtctctgagccccggcaag

SEQ ID NO: 85 - DNA, 681 bp, Artificial Sequence
  FcTregV25
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacggcggactggttcaacctggcggctctctgagactgtcttgtgccgccagcggcttcaccttcgagtacaagtgcaaggtgtccaacaaggccctgcctgctcctatcgagaaaaccatcagcaaggccaagggcgaagaacagtaccagagcacctacagagtggtgtccgtgctgacagtgctgcaccaggattgggtgtccctgacatgcctggtcaagggcttctacccttccgatatcgccgtggaatgggagagcaatggccagcctgagaacaactacaagaccacacctcctgtgctggacagcggcacagatttcaccctgaccatctctagcctgcagcctgaggatagatggcagcagggcaatgtgttcagctgctctgtgatgcacgaggccctgcacaaccactacacccagaagtctctgtctctgagccccggcaag

SEQ ID NO: 86 - DNA, 744 bp, Artificial Sequence
  FcTregV26
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtaccagagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagaccacacctcctgtgctggacagcggcacagatttcaccctgaccatctctagcctgcagcctgaggatagatggcagcagggcaatgtgttcagctgctctgtgatgcacgaggccctgcacaaccactacacccagaagtctctgtctctgagccccggcaaagaagaacagtatcagtccacctaccgggtcgtcagcgttctgaccgttctgcatcaggattgg

SEQ ID NO: 87 - DNA, 744 bp, Artificial Sequence
  FcTregV27
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtgggcaccgacttcaccctgaccatcagttctctgcagcccgaggacgagtacaagtgcaaggtgtccaacaaggccctgcctgctcctatcgagaaaaccatctctaaggctggcggactggtgcagcctggtggatctctgagactgtcttgtgccgccagcggcttcaccttcaatcaggtgtccctgacctgcctggtcaagggcttctacccttccgatatcgccgtggaatgggagagcaatggccagcctgagaacaactacaagaccacacctcctgtgctggacagcggcacagatttcacactgacgatctccagcctgcagcctgaagatagatggcagcagggcaacgtgttcagctgctctgtgatgcacgaggccctgcacaaccactacacccagaagtctctgtctctgagccctggaaaaggcggacttgttcaacctggcggaagcctgagactgagctgtgctgcctccggctttaccttc

SEQ ID NO: 88 - DNA, 744 bp, Artificial Sequence
  FcTregV28
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccctgtacctgcagatgaacagcctgagagccgaggacacagctaagcaccctccaagcagagatgagctgaccaagaaccaggtgtccctgacctgcctggtcaagggcttctacccttccgatatcgccgtggaatgggagagcaatggccagccagagaacaactacaagaccacacctcctgtgctggacagcgacggctcattcttcctgtacagcaagctgacagtggacaagagcagatggcagcagggcaacgtgttcagctgttctgtgatgcacgaggccctgcacaaccactacacccagaagtctctgtctctgagccccggcaaagaagaacagtaccagagcacataccgggtcgtcagcgtcctgacagtcctgcaccaagactgg

SEQ ID NO: 89 - DNA, 726 bp, Artificial Sequence
  FcTregV29
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagacactgtacctgcagatgaacagcctgagagccgaggataccgccaaacacgtgctgcaccaggactggctgaacggcaaagagtacaagtgcaaggtgtccaacaaggccctgcctgctcctatcgagaaaaccatcagcaaggccaagggcgaagaacagtaccagagcacctacagagtggtgtccgtgctgaccgtcctgcatcaggattgggtgtccctgacatgcctggtcaagggcttctacccttccgatatcgccgtggaatgggagagcaatggccagccagagaacaactacaagaccacacctcctgtgctggacagcgacggctctaaaaccctgtatctccagatgaattccctgcgcgccgaggacacagctaaacatgtgttcagctgcagcgtgatgcacgaggccctgcacaaccactacacccagaagtctctgtctctgagccctggcaagggcaccgatttcaccctgaccatttctagcctgcagcctgaggac

SEQ ID NO: 90 - DNA, 735 bp, Artificial Sequence
  FcTregV30
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagacactgtacctgcagatgaacagcctgagagccgaggataccgccaaacacgtgctgcaccaggactggctgaacggcaaagagtacaagtgcaaggtgtccaacaaggccctgcctgctcctatcgagaaaaccctgtatctccagatgaattccctgcgcgccgaggacacagctaagcaccctccatctagagatgagctgaccaagaaccaggtgtccctgacctgcctggtcaagggcttctacccttccgatatcgccgtggaatgggagagcaatggccagccagagaacaactacaagaccacacctcctgtgctggacagcggcacagatttcaccctgaccatctctagcctgcagcctgaggatagatggcagcagggcaatgtgttcagctgctctgtgatgcacgaggccctgcacaaccactacacccagaagtctctgtctctgagccccggcaagaaaactctctatctgcaaatgaactctctccgagctgaagatacggccaagcac

SEQ ID NO: 91 - DNA, 726 bp, Artificial Sequence
  FcTregV31
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtgggcaccgacttcaccctgaccatcagttctctgcagcccgaggacgagtacaagtgcaaggtgtccaacaaggccctgcctgctcctatcgagaaaaccctgtacctgcagatgaacagcctgagagccgaggacacagctaagcaccctccaagcagagatgagctgaccaagaaccaggtgtccctgacctgcctggtcaagggcttctacccttccgatatcgccgtggaatgggagagcaatggccagccagagaacaactacaagaccacaggtggactggtgcagcctggcggatctctgagactgtcttgtgccgccagcggcttcacctttagatggcagcagggcaacgtgttcagctgctctgtgatgcacgaggccctgcacaaccactacacccagaagtctctgtctctgagccctggcaagggcacagatttcacactgacgatctccagcctgcaacctgaggac

SEQ ID NO: 92 - DNA, 738 bp, Artificial Sequence
  FcTregV32
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacggcggactggttcaacctggcggctctctgagactgtcttgtgccgccagcggcttcaccttcgagtacaagtgcaaggtgtccaacaaggccctgcctgctcctatcgagaaaaccatcagcaaggccaagggcaccgacttcaccctgaccattagtagcctgcagcctgaggacagggacgagctgacaaagaatcaggtgtccctgacctgcctggtcaagggcttctacccttccgatatcgccgtggaatgggagagcaatggccagcctgagaacaactacaagaccacaggtggactggtgcagccaggtggaagcctgagacttagctgtgccgcctctggctttaccttcagatggcagcagggcaacgtgttcagctgctctgtgatgcacgaggccctgcacaaccactacacccagaagtctctgtctctgagccccggcaagaaaaccctgtacctgcagatgaactccctgagagccgaggacaccgccaagcac

SEQ ID NO: 93 - DNA, 735 bp, Artificial Sequence
  FcTregV33
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtgggcaccgacttcaccctgaccatcagttctctgcagcccgaggacgagtacaagtgcaaggtgtccaacaaggccctgcctgctcctatcgagaaaaccatcagcaaggccaagggcacagatttcacgctcaccatctctagcctgcagcctgaagatgacgagctgaccaagaaccaggtgtccctgacatgcctggtcaagggcttctacccttccgatatcgccgtggaatgggagagcaatggccagcctgagaacaactacaagaccacacctcctgtgctggacagcgacggcagcaagacactgtacctgcagatgaactccctgagagccgaggataccgccaagcacgtgttcagctgttctgtgatgcacgaggccctgcacaaccactacacccagaagtctctgtctctgagccccggcaagaaaaccctgtatctccagatgaatagcctgcgcgccgaggacacagccaaacac

SEQ ID NO: 94 - DNA, 735 bp, Artificial Sequence
  FcTregV34
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagaccacacctcctgaagaacagtaccagtccacctaccgggtcgtcagcgttctgacagttctgcatcaagactggcagcagggcaacgtgttcagctgttctgtgatgcacgaggccctgcacaaccactacacccagaagtctctgtctctgagccccggcaagaaaaccctgtacctgcagatgaactccctgagagccgaggacaccgccaagcac

SEQ ID NO: 95 - DNA, 744 bp, Artificial Sequence
  FcTregV35
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagacactgtacctgcagatgaacagcctgagagccgaggataccgccaaacacgtgctgcaccaggactggctgaacggcaaagagtacaagtgcaaggtgtccaacaaggccctgcctgctcctatcgagaaaaccatctctaaggctggcggcctggttcagcctggtggatctctgagactgtcttgtgccgccagcggcttcaccttcaatcaggtgtccctgacctgcctggtcaagggcttctacccttccgatatcgccgtggaatgggagagcaatggccagccagagaacaactacaagaccaccggcggacttgtgcagccaggtggaagtcttagactgagctgtgccgcctctggctttacctttagatggcagcagggcaacgtgttcagctgctctgtgatgcacgaggccctgcacaaccactacacccagaagtctctgtctctgagccctggcaaagaggaacagtaccagagcacctacagagtggtgtccgtgctgaccgtcctgcatcaggattgg

SEQ ID NO: 96 - DNA, 735 bp, Artificial Sequence
  FcTregV36
gataagacacacacatgtcctccatgtcctgctccagagctgctcggcggaccttccgttttcctgtttccacctaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagacactgtacctgcagatgaacagcctgagagccgaggataccgccaaacacgtgctgcaccaggactggctgaacggcaaagagtacaagtgcaaggtgtccaacaaggccctgcctgctcctatcgagggcaccgatttcaccctgaccatctctagcctgcagcctgaggaccctcctagcagagatgagctgaccaagaaccaggtgtccctgacctgcctggtcaagggcttctacccttccgatatcgccgtggaatgggagagcaatggccagccagagaacaactacaagaccacacctcctgtgctggacagcgacggctcattcttcctgtacagcaagctgaccgtggacaagagcagatggcagcagggcaatgtgttcagctgctctgtgatgcacgaggccctgcacaaccactacacccagaagtctctgtctctgagccctggcaaaggcggacttgttcaacctggcggctctctgagactgtcttgtgccgccagcggcttcaccttc

SEQ ID NO: 97 - protein, 88 aa, Artificial Sequence
  SIGNAL 1..20: Murine IgG signal peptide
  Dir-Treg01-FLAG
METDTLLLWVLLLWVPGSTGEEQYQSTYRVVSVLTVLHQDWGTDFTLTISSLQPEDLNNFYPREAKVQWKVDNALQSGNSDYKDDDDK

SEQ ID NO: 98 - protein, 82 aa, Artificial Sequence
  SIGNAL 1..20: signal
  Dir-Treg02-FLAG
METDTLLLWVLLLWVPGSTGGGLVQPGGSLRLSCAASGFTFKTLYLQMNSLRAEDTAKHGTDFTLTISSLQPEDDYKDDDDK

SEQ ID NO: 99 - protein, 88 aa, Artificial Sequence
  SIGNAL 1..20: Murine IgG signal peptide
  Dir-Treg03-FLAG
METDTLLLWVLLLWVPGSTGKTLYLQMNSLRAEDTAKHEEQYQSTYRVVSVLTVLHQDWGGLVQPGGSLRLSCAASGFTFDYKDDDDK

SEQ ID NO: 100 - protein, 8 aa, Artificial Sequence
  FLAG-TAG
DYKDDDDK

SEQ ID NO: 101 - DNA, 264 bp, Artificial Sequence
  Dir-Treg01-FLAG DNA
atggaaaccgacaccctgctgctgtgggtgctgctgctgtgggtgccaggatctaccggcgaggaacagtaccagagcacctaccgggtggtgtccgtgctgacagtgctgcaccaggactggggcaccgacttcaccctgaccatcagcagcctgcagcccgaggacctgaacaacttctacccccgcgaggccaaggtgcagtggaaggtggacaatgccctgcagagcggcaacagcgactacaaggacgacgacgacaag

SEQ ID NO: 102 - DNA, 246 bp, Artificial Sequence
  Dir-Treg02-FLAG DNA
atggaaaccgacaccctgctgctgtgggtgctgctgctgtgggtgccaggatctacaggcggaggactggtgcagcctggcggctctctgagactgtcttgtgccgccagcggcttcaccttcaagaccctgtacctgcagatgaacagcctgcgggccgaggacacagccaagcacggcaccgatttcaccctgaccatcagcagcctgcagcctgaggacgactacaaggacgacgacgacaag

SEQ ID NO: 103 - DNA, 264 bp, Artificial Sequence
  Dir-Treg03-FLAG DNA
atggaaaccgacaccctgctgctgtgggtgctgctgctgtgggtgccaggcagcacaggcaagaccctgtacctgcagatgaacagcctgcgggccgaggacacagccaagcacgaggaacagtaccagagcacctaccgggtggtgtccgtgctgacagtgctgcaccaggattggggcggactggtgcagcctggcggaagcctgagactgtcttgtgccgccagcggcttcaccttcgactacaaggacgacgacgacaag

SEQ ID NO: 104 - protein, 71 aa, Homo sapiens
SHEDPEVKFNWYVDGVEVHNAKTKPREEQYNSTYRVVSVLTVLHQDWLNGKEYKCKVSNKALPAPIEKTIS

SEQ ID NO: 105 - protein, 70 aa, Homo sapiens
LTKNQVSLTCLVKGFYPSDIAVEWESNGQPENNYKTTPPVLDSDGSFFLYSKLTVDKSRWQQGNVFSCSV

SEQ ID NO: 106 - protein, 47 aa, Homo sapiens
CKVSNKALPAPIEKTISKAKGQPREPQVYTLPPSRDELTKNQVSLTC

SEQ ID NO: 107 - protein, 7 aa, Artificial Sequence
  Linker 1
AGPGPSG

SEQ ID NO: 108 - protein, 5 aa, Artificial Sequence
  Linker 2
PTGSG

SEQ ID NO: 109 - protein, 5 aa, Artificial Sequence
  Linker 3
GGSTG

SEQ ID NO: 110 - protein, 4 aa, Artificial Sequence
  sequence or partial sequence of GS linker (GGSG)n
GGSG

SEQ ID NO: 111 - protein, 245 aa, Artificial Sequence
  FcTregV32_variant
DKTHTCPPCPAPELLGGPSVFLFPPKPKDTLMISRTPEVTCVVVDVSHEDPEVKFNWYVDGVEVHNAKTKPREEQYGGLVQPGGSLRLSCAASGFTFEYKCKVSNKALPAPIEKTISKAKGTDFTLTISSLQPEDDELTKNQVSLTCLVKGFYPSDIAVEWESNGQPENNYKTTGGLVQPGGSLRLSCAASGFTFRWQQGNVFSCSVMHEALHNHYTQKSLSLSPGKKTLYLQMNSLRAEDTAKH